(19) United States
(12) Patent Application Publication
(10) Pub. No.: US 2023/0330134 A1
(43) Pub. Date: Oct. 19, 2023

(54) METHOD OF PROPHYLAXIS OF CORONAVIRUS AND/OR RESPIRATORY SYNCYTIAL VIRUS INFECTION

(71) Applicant: Starpharma Pty Ltd., Abbotsford, Victoria (AU)

(72) Inventors: Jessica Huberman, Abbotsford (AU); Graham Heery, Abbotsford, Victoria (AU); Jacinth Fairley, Abbotsford (AU); Jeremy Robert Arthur Paull, Abbotsford (AU); Yao Da Dong, Clayton (AU)

(21) Appl. No.: 17/919,155

(22) PCT Filed: Apr. 14, 2021
(86) PCT No.: PCT/AU2021/050333

§ 371 Date: Oct. 14, 2022

(30) Foreign Application Priority Data
Apr. 15, 2020 (AU) 2020901194
Aug. 21, 2020 (AU) 2020902993
Nov. 17, 2020 (AU) 2020904246

(51) Int. Cl. A61K 31/795; A61K 9/00; A61P 31/14; A61K 47/34; A61K 47/38; A61K 47/10; A61K 47/14; A61K 47/18; A61K 45/06; A01N 37/20; A01P 1/00

(52) U.S. Cl. CPC A61K 31/795; A01N 37/20; A01P 1/00; A61K 9/0043; A61K 45/06; A61K 47/10; A61K 47/14; A61K 47/34; A61K 47/38; A61K 47/183; A61K 47/186; A61P 31/14

(57) ABSTRACT

The present invention relates to methods and compositions for preventing or reducing the likelihood of Coronavirus (CoV) and/or Respiratory syncytial virus (RSV) infection in an individual, preventing or reducing the likelihood or severity of a symptom associated with a CoV and/or RSV infection in an individual, reducing the severity and/or duration of a CoV and/or RSV infection in an individual, or treating a CoV and/or RSV infection in an individual, preventing or reducing viral shedding in an individual infected with a CoV and/or RSV infection, or reducing transmission of a CoV and/or RSV in a population comprising administering to the individual an effective amount of a macromolecule wherein the macromolecule comprises a dendrimer of 3-5 generations with one or more sulfonic acid or sulfonate containing moieties attached to one or more surface groups. The present invention also relates to a device for delivering a composition comprising such a macromolecule.

### FIELD OF THE INVENTION

The present invention relates to methods and compositions for preventing or reducing the likelihood of Coronavirus (CoV) and/or Respiratory syncytial virus (RSV) infection in an individual, preventing or reducing the likelihood or severity of a symptom associated with a CoV and/or RSV infection in an individual, reducing the severity and/or duration of a CoV and/or RSV infection in an individual, or treating a CoV and/or RSV infection in an individual, preventing or reducing viral shedding in an individual infected with a CoV and/or RSV infection, or reducing transmission of a CoV and/or RSV in a population comprising administering to the individual an effective amount of a macromolecule. The present invention also relates to a device for delivering a composition comprising a macromolecule.

### BACKGROUND OF THE INVENTION

Viral respiratory tract infections (VRTIs) are some of the most common infections worldwide and represent a major public health concern. Respiratory viruses cause infections in all age groups and are a major contributing factor to morbidity and mortality. Disease severity can range from mild, common cold-like illness to severe, life-threatening respiratory tract infection. The burden of VRTIs is often more pronounced in individuals with chronic co-morbidities or clinical risk factors.

In the past, a significant proportion of respiratory tract disease could not be attributed to a specific pathogen. With the advent of molecular detection and genotyping techniques, there has been a substantial increase in the recognition of several newly identified non-influenza respiratory viruses involved in disease.

These potential pathogens have included coronaviruses, adenovirus, rhinovirus species, human respiratory syncytial virus, and human bocaviruses. Coronaviruses (CoVs) are ubiquitous worldwide and are associated with relatively mild respiratory disease (e.g., the common cold) through to the emergence of severe acute respiratory syndrome (SARS).

Coronaviruses are large, enveloped viruses with a positive sense, single-stranded RNA genome. CoV infections are a serious threat to both humans and animals; they cause enzootic infections and are responsible for outbreaks of SARS caused by SARS-CoV, Middle-East respiratory syndrome (MERS) caused by MERS-CoV and coronavirus disease 2019 (COVID-19) caused by SARS-CoV-2 in humans. COVID-19 is the disease caused by the newly discovered SARS-CoV-2. Some people with SARS-CoV-2 infection remain asymptomatic, whilst in others, the infection can cause mild to moderate COVID-19 disease and COVID-19 pneumonia, leading some patients to require intensive care support and, in some cases, to death, especially in older adults. Symptoms such as fever, cough and loss of taste, and signs such as oxygen saturation or lung auscultation findings, are the first and most readily available diagnostic information.

In humans, CoVs typically cause acute respiratory infections. Symptoms and severity can range from mild upper respiratory infections (e.g. a common cold) to much more severe acute respiratory distress syndrome (ARDS), pneumonia, to single and multi-organ failures. Part of human CoV virulence is attributed to long incubation periods and the display of no or often mild symptoms by infected and infectious persons, meaning that many people do not realise they have been infected and continue their routines, thereby spreading infection.

Transmission of CoV is usually via airborne droplets to the nasal mucosa, where the virus then invades the respiratory tract. It is also possible that contaminated droplets on the hands may be transmitted to the oral and/or nasal mucosa. Currently, hygiene practices are recommended to prevent transmission and the disease is treated by symptom management. Mild symptoms, such as that of the common cold, are usually treated with nonsteroidal anti-inflammatory drugs. Vaccines have become commercially available since the filing of the provisional filing of this application and have commenced distribution. While a substantial number of potential medications have been proposed based on previous work on SARS-CoV, and some initial clinical testing has taken place, currently, no medication has been proven highly effective to treat infection by SARS-CoV-2. SARS-CoV-2 Spike S protein binds the ACE2 receptor for viral entry and it is believed that PIKfyve, TPC2, and cathepsin L are also critical for entry. In a recent study from UCSD, 332 high confidence SARS-CoV-2-human protein-protein interactions and 66 druggable human proteins or host factors targeted by 69 existing FDA-approved drugs, drugs in clinical trials and/or preclinical compounds, were identified. In addition, there are a wide range of drugs in development and testing, or tested against SARS-CoV-2, for example, neutralising antibodies against GM-CSF, IL-6R, CCR5, S protein of MERS, and drugs including, Remdesivir, ribavirin, tilorone, favipiravir, Kaletra (lopinavir / ritonavir), Prezcobix (darunavir/cobicistat), nelfinavir, mycophenolic acid, Galidesivir, Actemra, OYA1, BPI-002, Ifenprodil, APN01, EIDD-2801, baricitinib, camostat mesylate, lycorine, Brilacidin, BX-25, and interferons, more specifically IFNβ. Several antiviral compounds have been used to treat COVID-19 and may reduce disease duration and infection index; however, due to poor efficacy (Solidarity Trial, WHO), cost and side effects the drugs are not widely used or approved by regulatory agencies.

Respiratory syncytial virus (RSV) is a respiratory virus that is a member of the family Pneumoviridae and infects most humans by the age of 2. In healthy adults, symptoms are mild but in some individuals symptoms can be severe (particularly in infants and the elderly) resulting in hospitalisation. RSV is responsible for more than 60% of acute respiratory infections in children worldwide. The virus can also make individuals susceptible to secondary bacterial infections like pneumonia or otitis media. In the United States, it is estimated that 11,000 to 17,000 adults die from RSV infection annually, with approximately 10 times that number of patients hospitalized annually. RSV infections in adults are usually not primary infections and are predominantly mild to moderate in severity unless patients have an underlying risk factor such as being immunocompromised, having an underlying chronic pulmonary or circulatory disease, living in a long-term care facility, or being frail. The mortality rate of RSV is as high as 30 to 100% due to RSV infection in solid organ and bone marrow transplant recipients, particularly when infection has occurred within a few days after transplant surgery. Those who are immunosuppressed or otherwise immunocompromised are at enhanced risk of severe RSV infections. RSV is the third greatest cause of influenza-like illness in the elderly. However, it is the second greatest cause of hospitalization.

After many years of research the current therapies for reducing the virus are limited to treating symptoms and an effective vaccine is yet to be developed. One of the challenges is that many candidate cellular receptors have been described for RSV entry, including annexin II, CX3 chemokine receptor 1, epidermal growth factor receptor (EGF), calcium-dependent lectins, Toll-like receptor 4, intercellular adhesion molecule 1 (ICAM-1), and nucleolin. Some receptors like EGF are purportedly used by only certain strains of RSV. Furthermore, RSV is a rapidly evolving virus, making vaccine development difficult, particularly as RSV is known to either evade or suppress B cell memory in humans.

Antiviral dendrimers have been developed with activity against HIV, HPV and HSV in selected animal models, see for example WO02/079299 and WO2007/045009. However, antiviral agents are generally selective in their action against viruses, due primarily to receptor specificity and mode of action. There are no approved broad-spectrum antiviral agents for broad classes of viral agents such as enveloped RNA viruses or negative stranded RNA viruses. Even within a family, such as Herpes viridae, agents effective against one virus are not usually effective for others, e.g. treatments against varicella, EBV or HSV are not mutually effective.

Accordingly, there remains a need for agents capable of preventing or reducing the spread of VRTIs, in particular CoVs and/or RSVs. There also remains a need for reducing severity and duration of disease for VRTIs, in particular COVs and/RSVs.

### SUMMARY OF THE INVENTION

The present inventors have found that the dendrimeric macromolecule, SPL7013, has activity against CoVs and RSVs in vitro. Accordingly, SPL7013 and structurally-related compounds will find utility in reducing the transmission of CoVs and/or RSVs, and in preventing or reducing the incidence, severity and duration of associated conditions. In an aspect, the present invention provides a method of preventing or reducing the likelihood of Coronavirus (CoV) and/or Respiratory syncytial virus (RSV) infection in an individual, comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or, reducing the likelihood or severity of a symptom associated with a Coronavirus (CoV) and/or Respiratory syncytial virus (RSV) infection in an individual comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or reducing the likelihood of Coronavirus (CoV) infection in an individual, comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or, reducing the likelihood or severity of a symptom associated with a Coronavirus (CoV) infection in an individual comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of reducing the severity and/or duration of a Coronavirus (CoV) infection in an individual, comprising,
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of treating a Coronavirus (CoV) infection in an individual comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or reducing viral shedding in an individual infected with a Coronavirus (CoV), comprising,
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of reducing transmission of a Coronavirus (CoV) in a population, comprising:
administering to the respiratory tract of a portion of the population an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 1 to 8 generations with one or more sulfonic acid- or sulfonate-containing moieties attached to one or more surface groups of the dendrimer.

In an aspect, the present invention provides a method of preventing or reducing the likelihood of Respiratory syncytial virus (RSV) infection in an individual, comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or, reducing the likelihood or severity of a symptom associated with a Respiratory syncytial virus (RSV) infection in an individual comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of reducing the severity and/or duration of a Respiratory syncytial virus (RSV) infection in an individual, comprising,
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of treating a Respiratory syncytial virus (RSV) infection in an individual comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of preventing or reducing viral shedding in an individual infected with a Respiratory syncytial virus (RSV), comprising,
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a method of reducing transmission of a Respiratory syncytial virus (RSV) in a population, comprising:
administering to the respiratory tract of a portion of the population an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 1 to 8 generations with one or more sulfonic acid- or sulfonate-containing moieties attached to one or more surface groups of the dendrimer.

In some embodiments, the CoV is selected from an/a Alphacoronavirus, Betacoronavirus, Gammacoronavirus and Deltacoronavirus. In some embodiments, the CoV is a Betacorinavirus.

In some embodiments, the CoV is SARS-CoV-2 or a subtype of variant thereof. In some embodiments, the CoV is SARS-CoV-2.

In some embodiments, the RSV is subtype A or subtype B or a subtype or variant thereof. In some embodiments, the RSV is subtype A.

In some embodiments, the dendrimer is 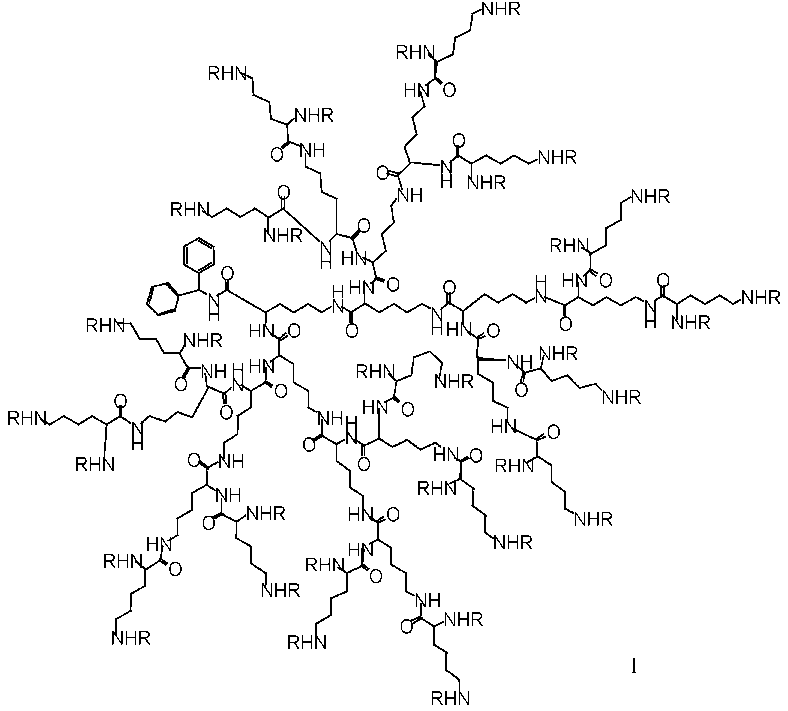 wherein at least 50% of R is

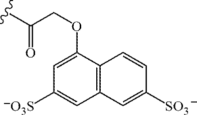

, and wherein the pharmaceutically acceptable salt is a sodium salt.

In an aspect, the present invention provides a composition for: preventing or reducing the likelihood of, or treating a Coronavirus (CoV) infection in an individual; reducing the severity and/or duration of CoV infection in an individual; preventing or reducing viral shedding in an individual infected with a COV; or reducing transmission of a CoV in a population, comprising:
an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

In an aspect, the present invention provides a device for delivering a nasal, oral or pulmonary composition comprising a macromolecule or a pharmaceutically acceptable salt thereof, or a composition comprising the macromolecule or pharmaceutically acceptable salt thereof and a pharmaceutically acceptable carrier,

wherein the macromolecule comprises a dendrimer of 3 to 5 generations with one or more sulfonic acid- or sulfonate-containing moieties attached to one or more surface groups of the dendrimer.

In an aspect, the present invention provides a composition comprising: an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 974 or Carbopol 971,
wherein the composition comprises a w/w ratio of about 1:20 to about 1:10 of Carpobol 974 or Carbopol 971 to the macromolecule.

In an aspect, the present invention provides a composition comprising: an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 974,
wherein the composition comprises about 0.05% w/w to about 5% w/w, or about 0.05% w/w to about 3% w/w, or about 0.05% w/w to about 2% w/w, or about 0.05% w/w to about 1% w/w, or about 0.05% w/w Carbopol 974.

In an aspect, the present invention provides a composition comprising: an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 971,
wherein the composition comprises about 0.05% w/w to about 1% w/w, or about 0.05% w/w to about 1.5% w/w, or about 0.05% w/w to about 1.8% w/w Carbopol 971.

In an aspect, the present invention provides a nasal moisture barrier dressing comprising a macromolecule or a pharmaceutically acceptable salt thereof, or a composition comprising the macromolecule or pharmaceutically acceptable salt thereof and a pharmaceutically acceptable carrier,

wherein the macromolecule comprises a dendrimer of 3 to 5 generations with one or more sulfonic acid- or sulfonate-containing moieties attached to one or more surface groups of the dendrimer

Any embodiment herein shall be taken to apply mutatis mutandis to any other embodiment unless specifically stated otherwise. For instance, as the skilled person would understand examples of macromolecules outlined above for the methods of the invention equally apply to compositions of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE DRAWINGS

FIG. 1 Provides the name and structures of the macromolecules SPL-7674, SPL-7615, SPL-7673, BAI-7021, BRI-2999, and BRI-2992.

FIG. 2 Shows the antiviral efficacy, measured by a reduction in cytopathic effect (CPE) in virus-infected cells, and selectivity of SPL7013 against SARS-CoV-2 (hCoV-19/Australia/VIC01/2020) infection of Vero E6 cells. The labels are as follows: EC₅₀=50% effective concentration; EC₉₀=90% effective concentration; CC₅₀=50% cytotoxic concentration; SI=selectivity index (CC₅₀/EC₅₀); SD=standard deviation; NC=not calculated; N/A=not applicable.

FIG. 3 Provides dose-response curves of the antiviral activity as measured by a reduction in CPE on Day 4 by SPL7013 against SARS-CoV-2 (hCoV-19/Australia/VIC01/2020) replication in Vero E6 cells, and cell viability as percent of cell control. A. Cell cultures infected one-hour pre-infection - Assay 1 (left panel) and Assay 2 (right panel). B. Cell cultures infected one-hour post-infection - Assay 1 (left panel) and Assay 2 (right panel).

FIG. 4 A. Virus and SPL7013 mixed for one hour prior to infection of cell cultures. EC₅₀ and CC₅₀ values and selectivity indices are indicated. Points and error bars represent mean ± SD of triplicate readings. B. The amount of virus secreted into the supernatant at 8 hours post-infection was determined by TCID₅₀. SPL7013 (0.345 mg/mL; squares), Remdesivir (5 µM; grey triangle), Hydroxychloroquine sulfate (15 µM; circles) and SARS-CoV-2 (hCoV-19/Australia/VIC01/2020) only (black triangles). Each point on the graph represents the virus titer present after one cycle of replication following addition of compound at the indicated time following virus infection. Infectious virus titer for SPL7013 was below the lower limit of detection (LLOD) at all time points.

FIG. 5 Shows dose-response and cytotoxicity analysis of SARS-CoV-2 (2019-nCoV/USA-WA1/2020) antiviral activity of SPL7013 in cells as measured by infectious virus release (Log₁₀ pfu/mL) on Day 4 post-infection in A. Vero E6 cells and B. Calu-3 cells. Points and error bars represent mean ± SD of triplicate readings.

FIG. 6 Provides the virucidal efficacy of SPL7013 against SARS-CoV-2 (2019-nCoV/USA-WA1/2020) measured by a reduction in mean infectious virus (Log₁₀ pfu/mL), at 96 hours post-infection in Vero E6 cells.

FIG. 7 Provides the virucidal efficacy of SPL7013 against SARS-CoV-2 (2019-nCoV/USA-WA1/2020) measured by a reduction in mean infectious virus (Log₁₀ pfu/mL), at 16 hours post-infection in Vero E6 cells. SPL7013 (0.0046 to 30 mg/mL) was incubated with 10⁵ and 10⁴ pfu/mL of SARS-CoV-2 (2019-nCoV/USA-WA1/2020) for 30 sec, 1 min, 5 min and 15 min. Treated virus was added to Vero E6 cells and the amount of infectious virus in the supernatant was determined by plaque assay 16 hours post-infection. A. Dose-response of SPL7013 virucidal activity using 10⁴ pfu/mL virus inoculum. Points and error bars represent mean ± SD of triplicate readings. B. Log₁₀ reduction (vs. baseline) in viral load with 10 mg/mL SPL7013. Columns and error bars represent mean ± SD of triplicate readings.

FIG. 8 A. Shows the assessment of SPL7013 against SARS-CoV-2 infection in hACE2 transgenic mice following nasal administration for 7 days. B. Shows inhibition of SARS-CoV, MERS-CoV and SARS-CoV-2 spike-expressing lentivirus infection of Vero E6 cells by SPL7013.

FIG. 9 A. Shows the inhibition of Human respiratory syncytial virus (HRSV) in Hep-2 cells following pre- and post-infection treatment with SPL7013. B. Shows the cytotoxicity of HRSV for Hep-2 cells following pre-treatment and post-treatment with SPL7013.

FIG. 10 Shows the antiviral efficacy of SPL7013 and iota-carrageenan against SARS-CoV-2 (2019-nCoV/USA-WA1/2020) measured by a reduction in nucleocapsid (ng/mL), at day 4 post-infection in human bronchial epithelial primary cells (HBEpC). Astodrimer sodium (0, 1.1, 3.3 and 10 mg/mL) or iota-carrageenan (0, 6, 60 and 600 µg/mL) were added to cell cultures 1 hour prior to infection. A. Shows the dose-response of SPL7013 antiviral activity. Points and error bars represent mean ± SD of triplicate readings. B. Shows the dose-response of carrageenan antiviral activity. Points represent one replicate. Dotted lines indicates level of inhibition achieved with positive control, SARS-CoV-2 pAb.

FIG. 11 A. RT-qPCR results in Vero E6 cells infected by SARS-CoV-2 Slovakia/SK-BMC5/2020 virus after treatment with SPL7013. All experiments were repeated once, independently (n=2). Results are expressed as percentage of RNA expression compared to the infected, non-treated control cells. B. Fluorescent foci of infection in Vero E6 cells infected by the SARS-CoV-2 Slovakia/SK-BMCS/2020 virus after treatment with SPL7013. All experiments were repeated once, independently (n=2). Titer was determined using the immunofluorescence foci assay.

FIG. 12 A. Viability of healthy Vero E6 cells after treatment with SPL7013. Cells were pre-incubated for 1h with SPL7013. All experiments were repeated once, independently (n=2). Viability was evaluated using the MTS Viability Assay. B. Viability of SARS-COV-2 Slovakia/SK-BMC5/2020 infected Vero E6 cells after treatment with SPL7013. Cells were pre-incubated for 1h with SPL7013 prior to infection with the virus. The virus was incubated with the cells for 48 hr. All experiments were repeated once, independently (n=2). Viability was evaluated using the MTS Viability Assay.

### DESCRIPTION OF THE INVENTION

### Definitions

The articles “a” and “an” are used herein to refer to one or to more than one (i.e. to at least one) of the grammatical object of the article. By way of example, “an element” means one element or more than one element.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “about” refers to a quantity, level, value, dimension, size, or amount that varies by as much as 30%, 25%, 20%, 15%, 10%, 5% or 1% to a reference quantity, level, value, dimension, size, or amount.

As used herein, the term “individual” refers to any individual susceptible to a CoV virus infection and/or RSV virus infection. In a particular embodiment, the individual is a human, including fetus, infant, child, early adult and adult. In some embodiments, the individual is a human adult. In one embodiment, the individual is an animal. In an embodiment, the child is one or more of: less than 16 years in age, less than 14 years in age, less than 12 years in age, less than 10 years in age, less than 5 years in age, less than 3 years in age, less than 2 years in age, less than 1 year in age, less than 6 months in age, less than three months in age, and less than one month in age. In an embodiment, the child is 12 years or older. In an embodiment, the infant is a preterm infant. In one embodiment, the adult is an elderly adult. In an embodiment, the adult is one or more of greater than 60 years in age, greater than 65 years in age, greater than 70 years in age, greater than 75 years in age, greater than 80 years in age, greater than 85 years in age, greater than 90 years in age. In some embodiments, the individual is a human. some embodiments, the individual is immunocompromised. In some embodiments, the individual has recently undergone surgery. In some embodiments, the individual is1 day, or 2 days, or 3 days, or 4 days, or 5 days, or 6 days, or 7 days, or 1.5 weeks, or 2 weeks, or three weeks post-surgery. In some embodiments, the individual is or will be a transplant recipient. In some embodiment, the individual is or will be a lung transplant recipient, or bone marrow or stem cell recipient. In some embodiments, the individual has a respiratory condition. In some embodiments, the respiratory condition is selected from one or more of: asthma, chronic obstructive pulmonary disease, sleep apnoea, emphysema, lung cancer, cystic fibrosis, bronchitis, chronic bronchitis, pneumonia, pleural effusion, whooping cough, COVID-19, asbestosis, bronchiectasis, pneumothorax, silicosis, and tuberculosis.

As used herein, the term “prevention” or “prophylaxis” refers to reducing the likelihood of contracting or developing infection or a symptom thereof. Prevention need not be complete and does not imply that a subject will not eventually contract or develop the infection or a symptom thereof.

As used herein, the terms “treating” or “treatment” refers to at least partially obtaining a desired therapeutic outcome. In an embodiment, treatment comprises preventing or delaying the appearance of one or more symptoms of a CoV and/or RSV infection. In an embodiment, treatment comprises arresting or reducing the development of one or more symptoms of a CoV and/or RSV infection.

As used herein, the phrase “reducing the severity of an infection”, or similar phrases, includes reducing one or more of the following in an individual: titer of a virus, duration of the virus infection, the harshness or duration of one or more symptoms of a virus infection in an individual. In an embodiment, the virus infection is a CoV and/or an RSV virus infection.

As used herein, the phrase “duration of a CoV and/or RSV infection” refers to the time in which an individual has a CoV and/or RSV infection or a symptom caused by a CoV and/or RSV infection.

As used herein, the phrase “macromolecules and pharmaceutically acceptable salts thereof” is used interchangeably with “macromolecules” as the context requires.

As used herein, “SPL7013” refers to astodrimer sodium (INN, USAN), CAS number 676271-69-5. SPL7013 is also known as 2, 6-Bis-{(1-napthaleny1-3,6-disulfonic acid)-oxyacetamido}-2,6-bis-2,6-bis-2,6-bis-(2,6-diamino-hexanoylamino)-2,6-diamino-hexanoic acid (diphenylmethyl)-amide, polysodium salt; or Tetrahexacontasodium N2,N6-bis {N2,N6-bis[N2,N6-bis(N2,N6-bis {N2,N6-bis[(3,6-disulfonatonaphthalen-1-y1oxy)acety1]-1-1ysy1}-1-1ysy1)-1-1ysy1]-1-1ysy1}-N1-(dipheny1methy1)-1-1ysinamide.

As used herein, “astodrimer” refers to CAS number 1379746-42-5. Also known as 2, 6-Bis-{(1-napthaleny1-3,6-disulfonic acid)-oxyacetamido}-2,6-bis-2,6-bis-2,6-bis-(2,6-diamino-hexanoylamino)-2,6-diamino-hexanoic acid (diphenylmethyl)-amide; or N2,N6-bis {N2,N6-bis[N2,N6-bis(N2,N6-bis {N2,N6-bis[(3,6-disulfonatonaphthalen-1-yloxy)acety1]-1-1ysy1)1-1ysy1)-1-lysy1]-1-1ysyl1-N1-(diphenylmethy1)-1-lysinamide.

### Macromolecules and Pharmaceutically Acceptable Salts Thereof

The present disclosure involves the use of macromolecules and/or pharmaceutically acceptable salts thereof. Given that the macromolecule may contain multiple sulfonate groups, the pharmaceutically acceptable salts may comprise multiple cations.

The pharmaceutically acceptable salt may be of any suitable type. Examples of suitable salts include, but are not limited to metallic salts (for example, aluminium, calcium, lithium, magnesium, potassium, sodium and zinc salts), organic salts (for example, organic amines such as N,NI-dibenzylethylenediamine, chloroprocaine, diethanolamine, ethylenediamine, dicyclohexylamine, cyclohexylamine, meglumine, (N-methylglucamine) and procaine), quaternary amines (for example, choline), sulphonium salts and phosphonium salts. In particular embodiments, salts are selected from sodium and potassium, especially sodium. In an embodiment, the salt is a sodium salt (e.g. it may be a polysodium salt).

Those skilled in the art will appreciate that many organic compounds can form complexes in solvents in which they are reacted or from which they are precipitated or crystallized. These complexes are known as “solvates”. For example, a complex with water is known as a “hydrate”. Solvates, such as hydrates, exist when the compound incorporates solvent. It will be understood that the macromolecules of the present invention, as well as salts thereof, may be present in the form of solvates. Solvates of the macromolecules which are suitable are those where the associated solvent is pharmaceutically acceptable.

The macromolecules used in the present invention comprise dendrimers of 3 to 5 generations with one or more sulfonic acid- or sulfonate-containing moieties attached to one or more surface groups of the dendrimer. The dendrimers useful in the invention may be any suitable dendrimer of 3 to 5 generations that is capable of presenting one or more sulfonic acid- or sulfonate-containing moieties on its surface. In some embodiments, the dendrimer is selected from a polylysine, polyglutamate, polyaspartate, polyamidoamine (PAMAM), poly(etherhydroxylamine), polyether, polyester or poly(propyleneimide) (PPI) dendrimer having 3 to 5 generations. In some embodiments, the dendrimer has 2 to 6 generations. In some embodiments, the dendrimer has 3 to 4 generations. In some embodiments, the dendrimer has 4 generations. In some embodiments the dendrimer is an amino acid dendrimer, selected from the group comprising polylysine, polyglutamate and polyaspartate.

The macromolecule also comprises one or more sulfonic acid- or sulfonate-containing moieties attached to the one or more surface functional groups of the outermost generation of the dendrimer. For example, when the dendrimer is a polylysine, polyamidoamine, poly(etherhydroxylamine) or poly(propyleneimide) dendrimer, the surface functional groups are amino groups, and when the dendrimer is a polyglutamate or polyaspartate dendrimer, the surface functional groups are carboxylic acids.

Dendrimers are branched polymeric macromolecules composed of multiple branched monomers radiating from a central core moiety. The number of branch points increases upon moving from the dendrimer core to its surface and is defined by successive layers or “generations” of monomers (or building units). Each generation of building units is numbered to indicate the distance from the core. For example, Generation 1 (G1) is the layer of building units attached to the core, Generation 2 (G2) is the layer of building units attached to Generation 1, Generation 3 (G3) is the layer of building units attached to Generation 2, and so on.

The outermost generation of building units provides the surface of the dendrimer and presents functional groups, to which the at least one sulfonic acid- or sulfonate-containing moiety is covalently bonded. The sulfonic acid- or sulfonate-containing group may be directly bonded to the surface functional group or may be attached to the surface functional group through a linker.

The dendrimers contemplated herein can be prepared by methods known in the art. For example, they may be prepared in either a convergent manner (where, effectively, the branches are pre-formed and then attached to the core) or a divergent manner (where the layers or generations are successively built outwards from the core). Both these methods would be well understood to the skilled person.

For example, in the case of lysine dendrimers, a divergent synthesis may involve reaction of the amine groups of a layer of lysine residues, with the carboxyl groups of amino-protected lysines, using amidation chemistry, to “grow” the dendrimer and form the next generation of building units. The protecting groups may then be removed, unveiling the amino groups of the new generation of lysine building units.

The dendrimers may comprise any suitable core. As used herein, “core” refers to the moiety upon which generations of monomers or building units are built (either through a divergent process or a convergent process), and may be any moiety having at least one reactive or functional site from which layers of monomer or building units are successively generated (or to which a pre-formed “branch” is attached).

The core may be formed from a core precursor having reactive groups suitable for reaction with building units, for example the core may be formed from a core precursor having 1, 2, 3 or 4 reactive groups. Some exemplary suitable cores contemplated herein include those formed from core precursors having 1, 2, 3 or 4 reactive groups independently selected from, amino, carboxyl, thiol, alkyl, alkynyl, nitrile, halo, azido, hydroxylamine, carbonyl, maleimide, acrylate or hydroxy groups to which the layers or generations of building units or monomers can be attached.

In some embodiments, the core is covalently attached to two building units via amide linkages, each amide linkage being formed between a nitrogen atom present in the core and the carbon atom of an acyl group present in a building unit. Accordingly, the core may for example be formed from a core precursor comprising two amino groups.

A Core Moiety May Be the Same as a Building Unit or May Be Different.

Exemplary cores include polyaminohydrocarbons, disulfide containing polyamines, poly(glycidyl ethers), aminoethanol, ammonia, arylmethylhalides, piperazine, aminoethylpiperazine, poly(ethyleneimine), alkylene/arylene dithiols, 4,4-dithiobutyric acid, mercaptoalkylamines, thioether alkylamines, isocyanurate, heterocycles, macrocycles, polyglycidylmethacrylate, phosphine, porphines, oxiranes, thioranes, oxetanes, aziridines, azetidines, multiazidofunctionalities, siloxanes, oxazolines, carbamates or caprolactones.

Some non-limiting examples of core moieties contemplated herein include ammonia and diamino C₂-C₁₂ alkanes such as ethylene diamine, 1,4-diaminobutane and 1,6-diaminohexane. However, it will be appreciated that the core is not necessarily a linear moiety with a single reactive group at each end. Non-linear, cyclic or branched core moieties are also contemplated by the present invention. For example, arylmethylamines such as benzhydrylamine (BHA), are suitable cores. In some embodiments, the core is or comprises a benzhydrylamine (BHA) group:

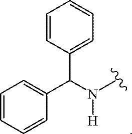

In some preferred embodiments, the core is a benzyhydrylamine-lysine core (BHALys). A BHALys core has the following structure:

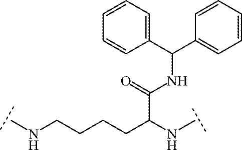 [SMILES: NCCCCC(N)C(=O)NC(c1ccccc1)c1ccccc1]

and, in the dendrimers, is covalently attached to building units through two nitrogen atoms. A BHALys core, may, for example, be formed from a core precursor:

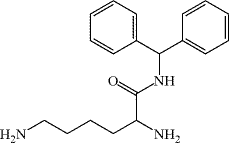

having two reactive amino nitrogens.

In some preferred embodiments, the core is BHALys core which comprises an L-lysine residue.

In some preferred embodiments, the core is BHALys core which contains an L-lysine residue.

The dendrimers also comprise one or more building units. In some embodiments, the building units of the dendrimer are selected from:
Lysine building units:

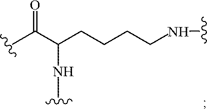

Amidoamine building units:

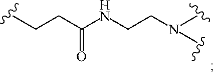

Etherhydroxyamine building units:

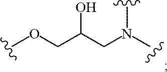

Propyleneimine building units:

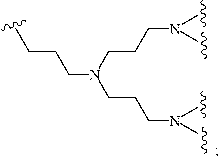

Glutamic acid building units:

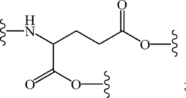

Aspartic acid building units:

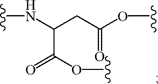

Polyester building units:

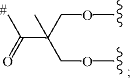

; and
Polyether building units:

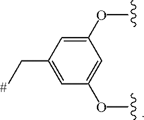

In some preferred embodiments, the building units are lysine residues, e.g.:

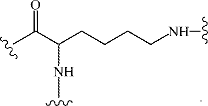

In some preferred embodiments, the building units are L-lysine residues, e.g.:

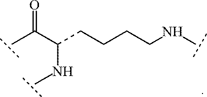

In some embodiments, the building unit or building units of the dendrimer are lysine or lysine analogues selected from a compound of the following formula:

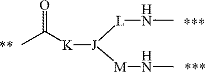

wherein K is absent or is selected from -C ₁₋₆ alkylene-, -C ₁₋₆ alkyleneNHC(O)-, -C₁₋₆ alkyleneC(O)-, -C₁₋₃ alkylene-O-C₁₋₃ alkylene-, -C₁₋₃ alkylene-O-C₁₋₃ alkyleneNHC(O)-and -C₁₋₃ alkylene-O-C₁₋₃ alkyleneC(O)-;
J is selected from CH or N;
L and M are independently absent or is selected from -C₁₋₆ alkylene- or -C₁₋₃ alkyleneOC₁₋₃ alkylene; provided that when L and/or M are absent, J is CH;
   ** indicates the linkage between the lysine or lysine analogue and the core of the dendrimer or the previous generation of building units; and
   *** indicates the linkage between the lysine or lysine analogue and the subsequent generation of lysine or lysine analogues or forms the surface amino groups of the dendrimer.

Exemplary lysine analogue building units including the following: Glycyl-Lysine 1 having the structure:

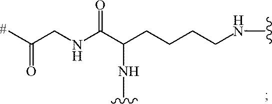

Analogue 2, having the structure below, where a is an integer 1 or 2; and b and c are independently integers 1, 2, 3 or 4:

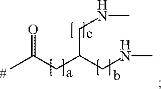

Analogue 3, having the structure below, where a is an integer 0, 1 or 2; and b and c are independently integers 2, 3, 4, 5 or 6:

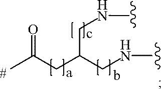

and Analogue 4, having the structure below, where a is an integer 0, 1, 2, 3, 4 or 5;and b and c are independently integers 1, 2, 3, 4 or 5:

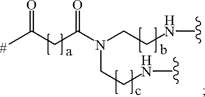

wherein each # denotes the carbonyl residue of the carboxyl group which forms an amide bond with a nitrogen atom of the core or a nitrogen atom of a previous generation of building units; and wherein any methylene group of the building units may be replaced by a methyleneoxy (CH₂—O) or ethyleneoxy (CH₂—CH₂—O) group, provided that this does not result in the formation of a carbonate (—O—C(O)—O—) or carbamate (—O—C(O)—N—) moiety within the building unit.

Other suitable building units/building unit precursors include:
Analogue 5, having the structure below, where a is an integer of 0 to 2; b and c are the same or different and are integers of 1 to 4; A₁ and A₂ are the same or different and selected from NH₂, CO₂H, OH, SH, X, Allyl-X, epoxide, aziridine, N₃ or alkyne, where X is F, Cl, Br or I,

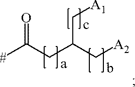

Analogue 6, having the structure below, where a is an integer of 0 to 2; b and c are the same or different and are integers of 2 to 6; A₁ and A₂ are the same or different and selected from NH₂, CO₂H, OH, SH, X, Allyl-X, epoxide, aziridine, N₃ or alkyne, where X is F, Cl, Br or I,

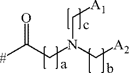

; and
Analogue 7, having the structure below, where a is an integer of 0 to 5; b and c are the same or different and are integers of 1 to 5; A₁ and A₂ are the same or different and selected from NH₂, CO₂H, OH, SH, X, Allyl-X, epoxide, aziridine, N₃ or alkyne, where X is F, Cl, Br or I,

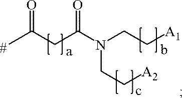

wherein each # denotes the carbonyl residue of the carboxyl group which forms an amide bond with a nitrogen atom of the core or a nitrogen atom of a previous generation of building units;
and wherein any methylene group of the building units may be replaced by a methyleneoxy (CH₂—O) or ethyleneoxy (CH₂—CH₂—O) group, provided that this does not result in the formation of a carbonate (—O—C(O)—O—) or carbamate (—O—C(O)—N—) moiety within the building unit.

In some embodiments, the macromolecule is a polylysine dendrimer having lysine building units, especially a polylysine dendrimer with a benzhydrylamine group, e.g. a dendrimer as shown below:

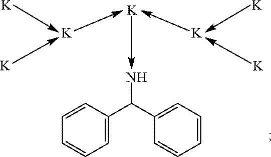

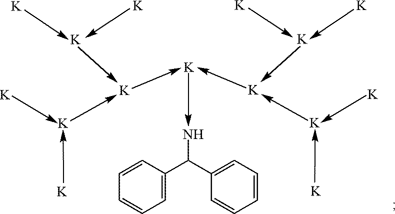

or

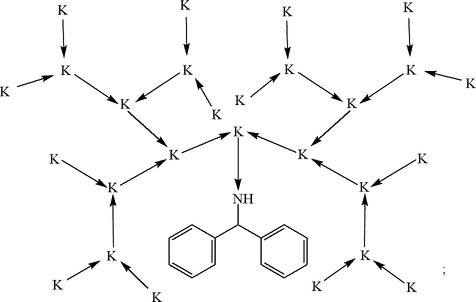

wherein

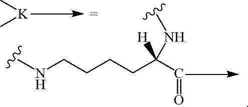

In some aspects the dendrimer contains 3 to 5 generations of building units, e.g. in some embodiments it comprises a core and 3 to 5 generation is of building units. In some embodiments, the dendrimer comprises a BHALys core and 3 to 5 generations of lysine building units. In some embodiments, the dendrimer provides 16, 32 or 64 nitrogen atoms on the surface layer of building units for attachment to sulfonic acid or sulfonate-containing moieties (either directly or via a linker). In some embodiments, the dendrimer provides 32 nitrogen atoms on the surface layer of building units for attachment to sulfonic acid or sulfonate-containing moieties (either directly or via a linker).

The sulfonic acid-containing or sulfonate-containing moiety is a moiety that is able to present the sulfonic acid or sulfonate group on the surface of the dendrimer. In some embodiments, the sulfonic acid- or sulfonate-containing moiety has one sulfonic acid or sulfonate group. In other embodiments, the sulfonic acid- or sulfonate-containing moiety has more than one sulfonic acid or sulfonate group, for example 2 or 3 sulfonic acid or sulfonate groups, especially 2 sulfonic acid or sulfonate groups. In some embodiments, the sulfonic acid- or sulfonate-containing moiety comprises an aryl group, such as a phenyl group or naphthyl group, especially a naphthyl group. In some embodiments, the sulfonic acid- or sulfonate-containing moiety comprises a naphthyl group substituted by two sulfonic acid or sulfonate moieties (also referred to as a napthyldisulfonate moiety), for example a 3,6-disulfonatonapthyl moiety. In some embodiments, a 3,6-disulfonatonapthyl moiety which is connected to the dendrimer through the 1-position of the naphthalene is used.

When the sulfonate-containing moiety is present, the moiety may be present in ionic form (-SO₃⁻) or in the form of a salt, for example, the sodium salt (-SO₃Na).

Examples of suitable sulfonic acid or sulfonate-containing moieties include but are not limited to:

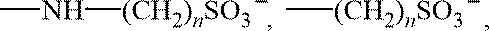

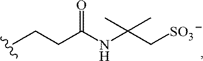

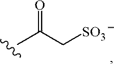

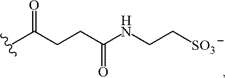

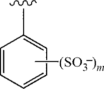

and

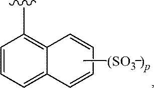

in which n is 0 or an integer of 1 to 20, m is an integer of 1 or 2 and p is an integer of 1 to 3. In some embodiments, p=2.

In some embodiments, the sulfonic acid- or sulfonate-containing moiety is selected from:

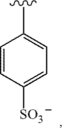

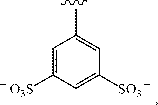

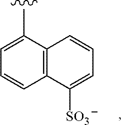

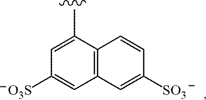

and

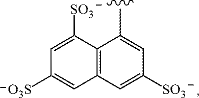

especially

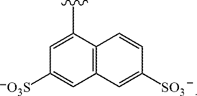

In some embodiments, more than one sulfonic acid- or sulfonate-containing moiety is present on the surface of the dendrimer. In some embodiments at least 5, at least 15 or at least 30 sulfonic acid- or sulfonate-containing moieties are present on the surface of the dendrimer. In some embodiments, 32 sulfonic acid- or sulfonate-containing moieties are present on the surface of the dendrimer.

In some embodiments, the sulfonic acid- or sulfonate-containing moiety is directly bonded to the surface amino group of the dendrimer. In other embodiments, the sulfonic acid- or sulfonate-containing moiety is attached to the surface amino group of the dendrimer through a linker group.

Suitable linker groups include straight chain and branched alkylene or alkenylene groups in which one or more non-adjacent carbon atoms is optionally replaced by an oxygen or sulfur atom to provide an ether, thioether, polyether or polythioether; or a group —X₁—(CH₂)_{q}—X₂ or —X₁—(CR₁R₂)_{q}—X₂—wherein X₁ and X₂ are independently selected from —NH—, —C(O)—, —O—, —S— and —C(S), R₁ and R₂ are independently selected from hydrogen or -C₁₋₆alkyl, and q is an integer from 1 to 10, and, where the linker comprises more than one CH₂ groups, optionally one or more non-adjacent (CH₂) groups may be replaced with —O— or —S— to form an ether, thioether, polyether or polythioether.

In some embodiments, the linker is a group —X₁—(CH₂)_{q-}C(O)—, wherein X₁ is the atom which is attached to the sulfonic acid- or sulfonate-containing moiety and is selected from the group consisting of O, NH and S; q is an integer of from 1 to 3; and the carbon of the —C(O)— group is attached to the surface amino group of the dendrimer.

In some embodiments, the linker is a group —X₁—( CR₁R₂)_{q} C(O)—, wherein X₁ is the atom which is attached to the sulfonic acid- or sulfonate-containing moiety and is selected from the group consisting of O, NH and S; R₁ and R₂ are independently selected from hydrogen or -C₁₋₆alkyl, q is an integer of from 1 to 3; and the carbon of the —C(O)— group is attached to the surface amino group of the dendrimer.

In some embodiments, the linker is

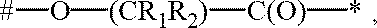

wherein Ri is —C₁—₆alkyl (e.g methyl, ethyl, propyl, butyl, pentyl or hexyl), R₂ is hydrogen, and in which # designates attachment to the sulfonic acid-containing moiety and * designates attachment to the surface amino group of the dendrimer.

In some embodiments, the linker is

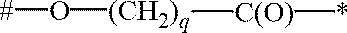

wherein q is an integer of from 1 to 6, and in which # designates attachment to the sulfonic acid-containing moiety and * designates attachment to the surface amino group of the dendrimer.

In a particular embodiment, the linker is

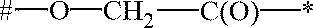

in which # designates attachment to the sulfonic acid-containing moiety and * designates attachment to the surface amino group of the dendrimer.

In some embodiments, the sulfonic acid- or sulfonate-containing moiety is attached to the surface amino group of the dendrimer through a linker group, and the linker-sulfonic acid/sulfonate moiety is:

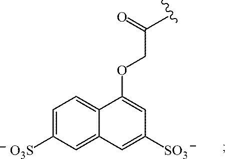

or a pharmaceutically acceptable salt thereof.

In some embodiments, the sulfonic acid- or sulfonate-containing moiety is

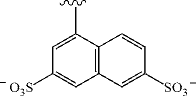

, and the linker is

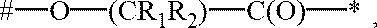

wherein R₁ is -C₁₋₆alkyl (e.g methyl, ethyl, propyl, butyl, pentyl or hexyl), R₂ is hydrogen, and in which # designates attachment to the sulfonic acid-containing moiety and * designates attachment to the surface amino group of the dendrimer.

In some embodiments, the sulfonic acid- or sulfonate-containing moiety is

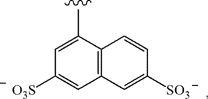

and the linker is

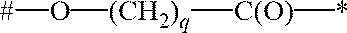

wherein q is an integer of from 1 to 6, and in which # designates attachment to the sulfonic acid-containing moiety and * designates attachment to the surface amino group of the dendrimer.

Exemplary dendrimers useful in the invention include those of formulae I, II and III:

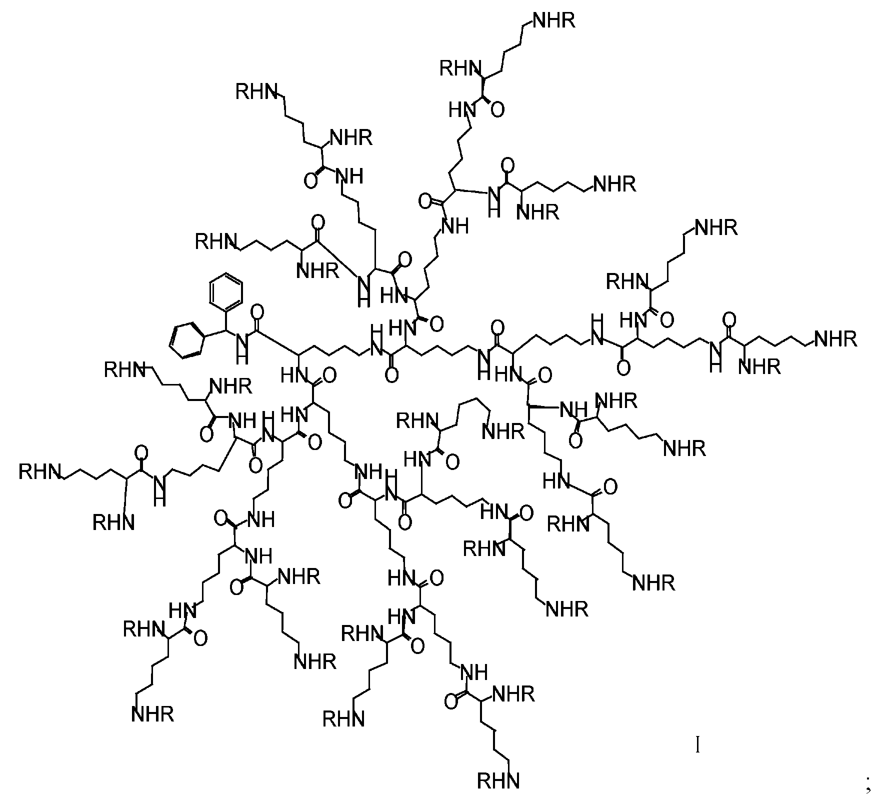

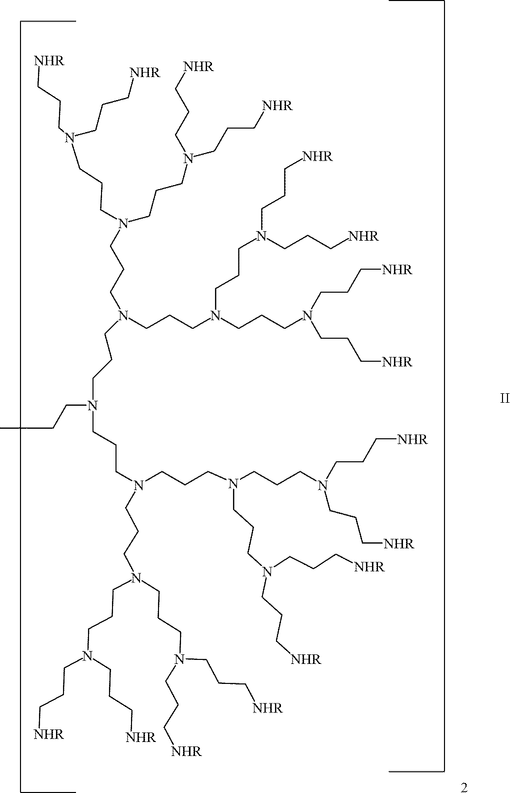

;

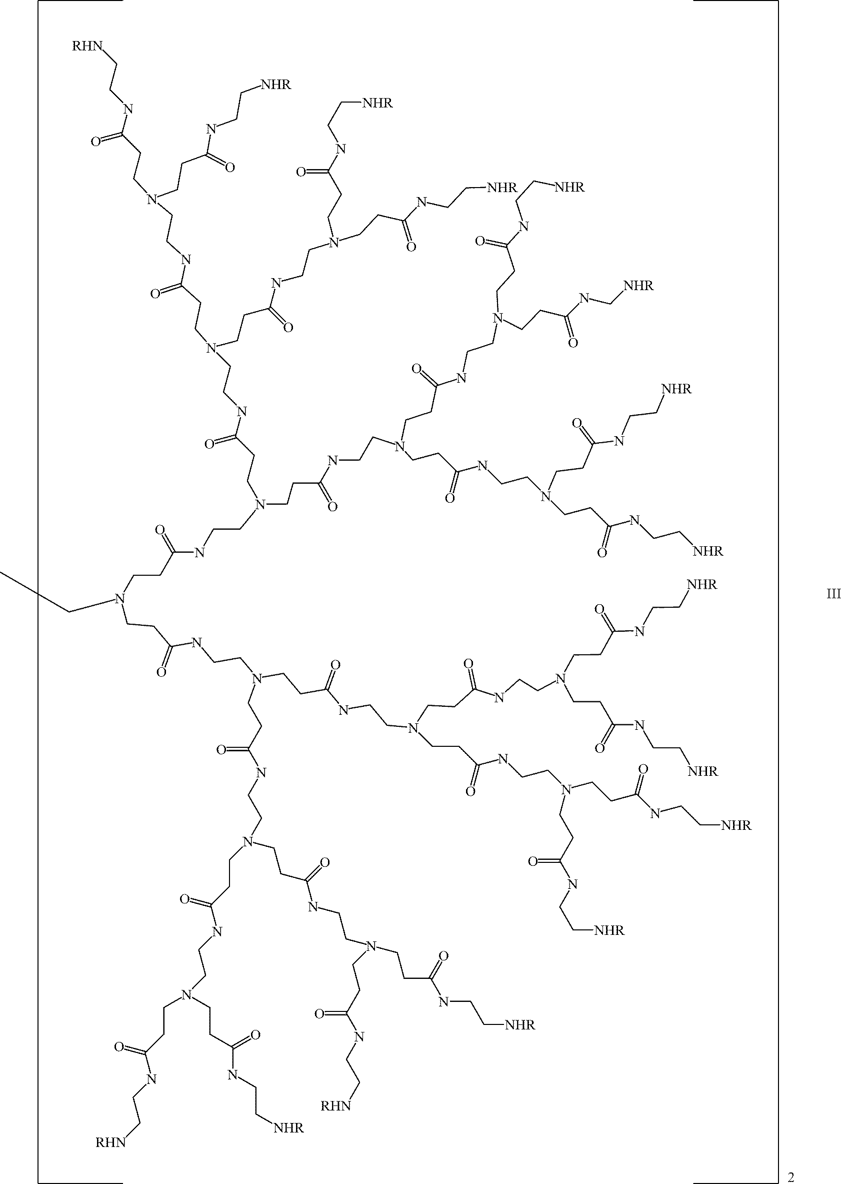

; in which each R group is represented by a group of formula IV or hydrogen:

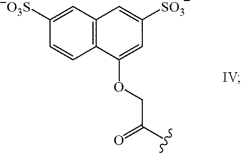

provided that at least one R group is a group of formula IV; or a pharmaceutically acceptable salt thereof.

In particular embodiments, more than one R group is a group of formula IV, for example in some embodiments at least 10 of the R groups are groups of formula IV, at least 15 of the R groups are groups of formula IV, at least 20 of the R groups are groups of formula IV, at least 25 of the R groups are groups of formula IV or at least 30 of the R groups are groups of formula IV. In some embodiments, all of the R groups are groups of formula IV.

In some embodiments, the dendrimer is 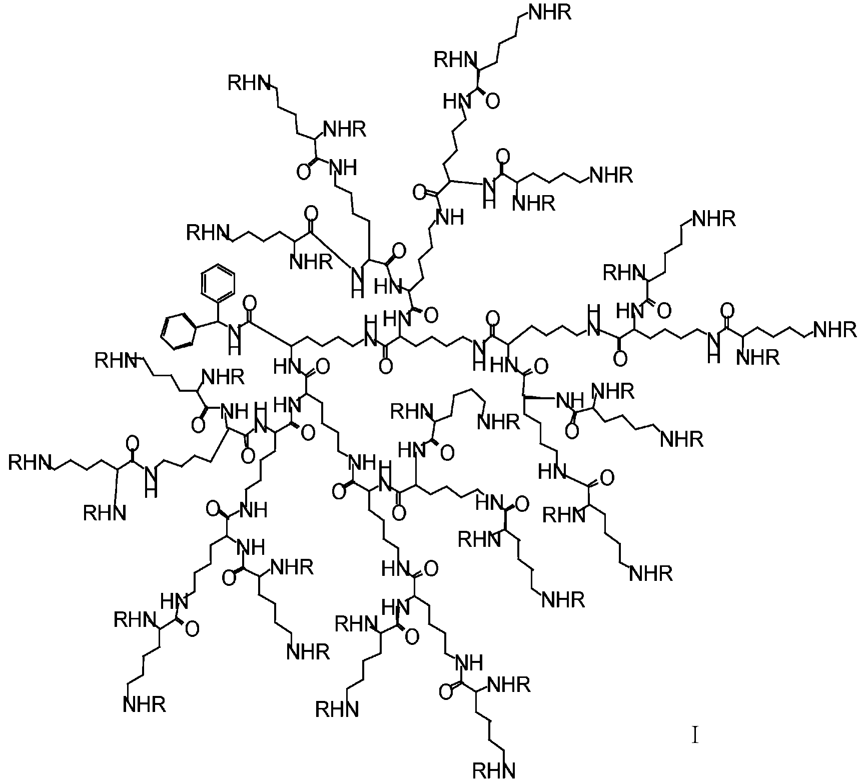 wherein at least 25% of R is

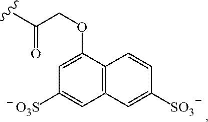

and wherein the pharmaceutically acceptable salt is a sodium salt.

In some embodiments, the dendrimer is 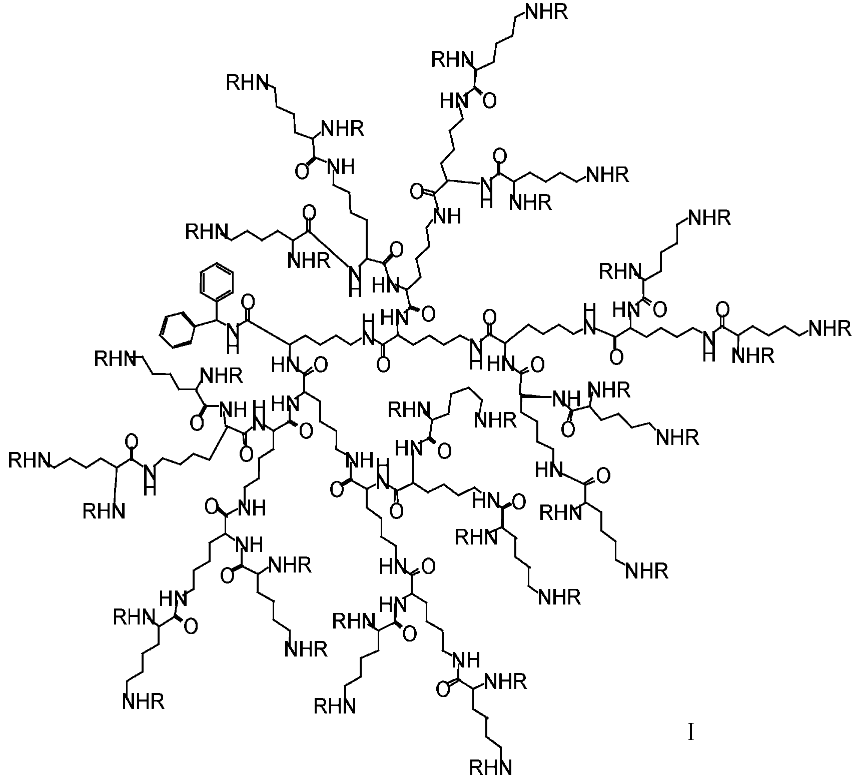 wherein R is hydrogen or

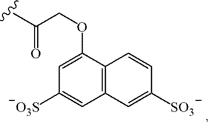

and wherein at least 25%, at least 50%, at least 75%, or at least 90% of R is

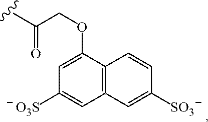

and wherein the pharmaceutically acceptable salt is a sodium salt.

In some embodiments, the macromolecule is a dendrimer of formula I: 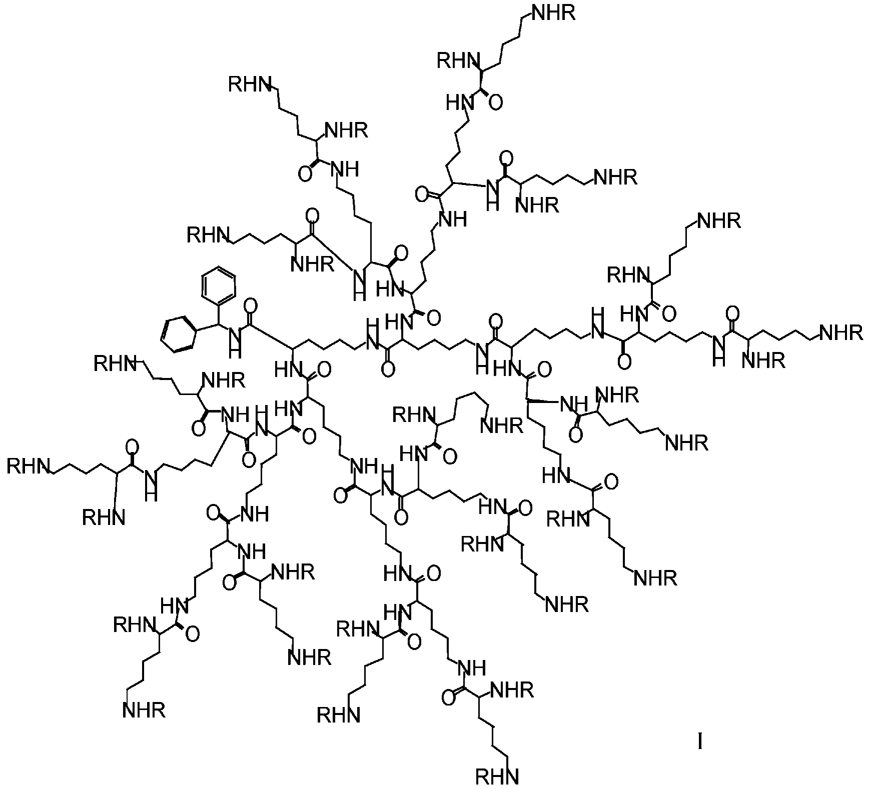 where R represents a group of the formula IV:

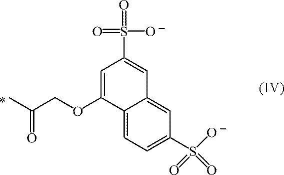

wherein * represents the attachment point to the surface amino group of the dendrimer, and wherein the pharmaceutically acceptable salt is sodium.

In some embodiments, the dendrimer is 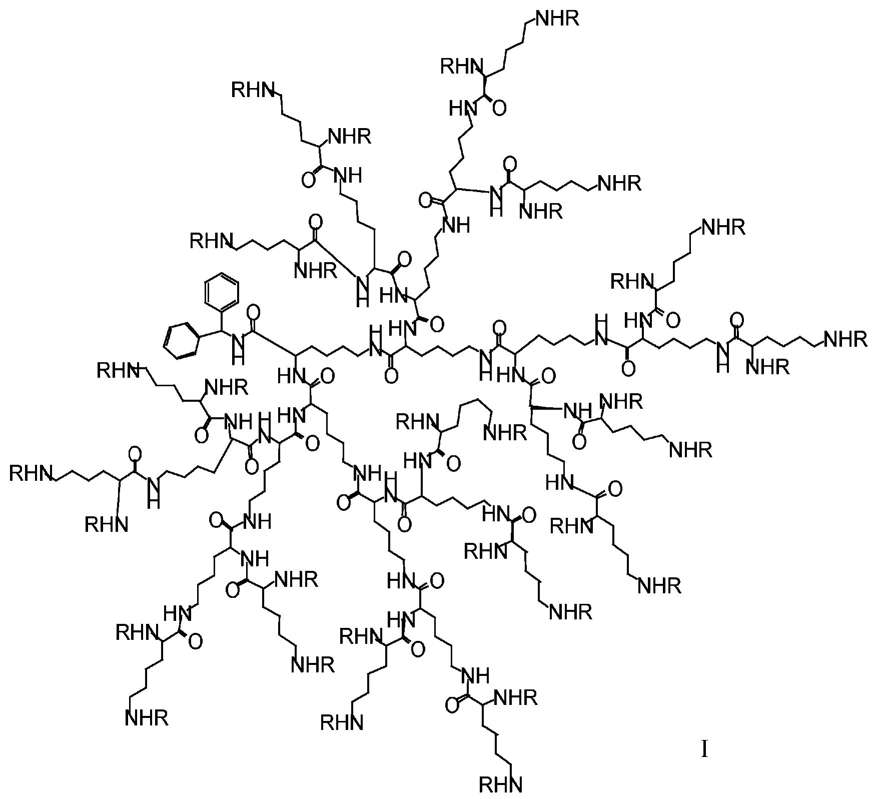 wherein R is hydrogen or a group R′, R′ is a linked sulfonic acid- or sulfonate-containing moiety, in which the sulfonic acid- or sulfonate-containing moiety is

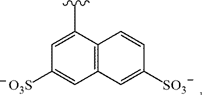

and the linker is

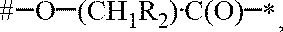

wherein R₁ is -C₁₋₆alkyl (e.g methyl, ethyl, propyl, butyl, pentyl or hexyl), R₂ is hydrogen, and in which # designates attachment to the sulfonic acid- or sulfonate-containing moiety and * designates attachment to the surface amino group of the dendrimer,
and wherein at least 25%, at least 50%, at least 75%, or at least 90%, or all, of R is R′, and wherein the pharmaceutically acceptable salt is a sodium salt.

In some embodiments, the dendrimer is

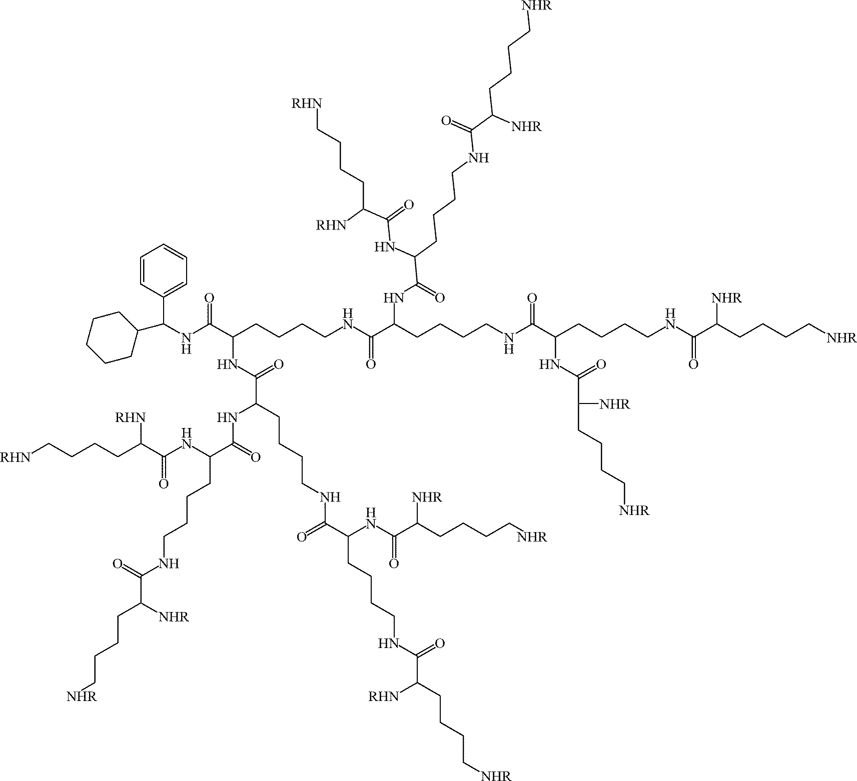

wherein R is hydrogen or a group R′,R′ is a linked sulfonic acid- or sulfonate-containing moiety, in which the sulfonic acid- or sulfonate-containing moiety is

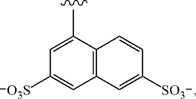

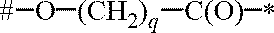

and the linker is wherein q is an integer of from 1 to 6, and in which # designates attachment to the sulfonic acid- or sulfonate-containing moiety and * designates attachment to the surface amino group of the dendrimer, and wherein at least 25%, at least 50%, at least 75%, or at least 90%, or all, of R is R′, and wherein the pharmaceutically acceptable salt is a sodium salt.

A particular dendrimer of formula I has all R groups as groups of formula IV (SPL7013). SPL7013, also known as astodrimer sodium, has the structure:

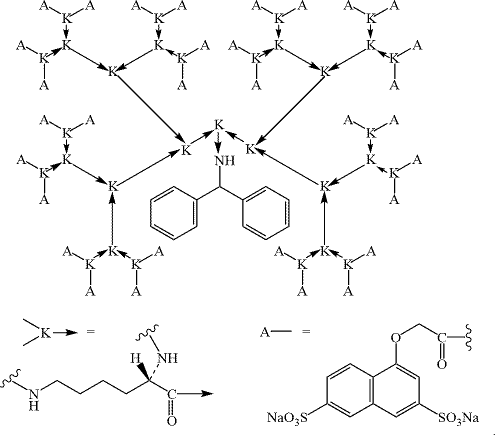

In some embodiments, the macromolecule is astodrimer. In some embodiments, the macromolecule is a pharmaceutically acceptable salt of astodrimer. In some embodiments, the pharmaceutically acceptable salt thereof is SPL7013 (astodrimer sodium).

A particular dendrimer of formula II has all R groups as groups of formula IV (SPL7320). A particular dendrimer of formula III has all R groups as groups of formula IV (SPL7304).

The synthesis of dendrimers of Formulae I, II and III is described in WO02/079299.

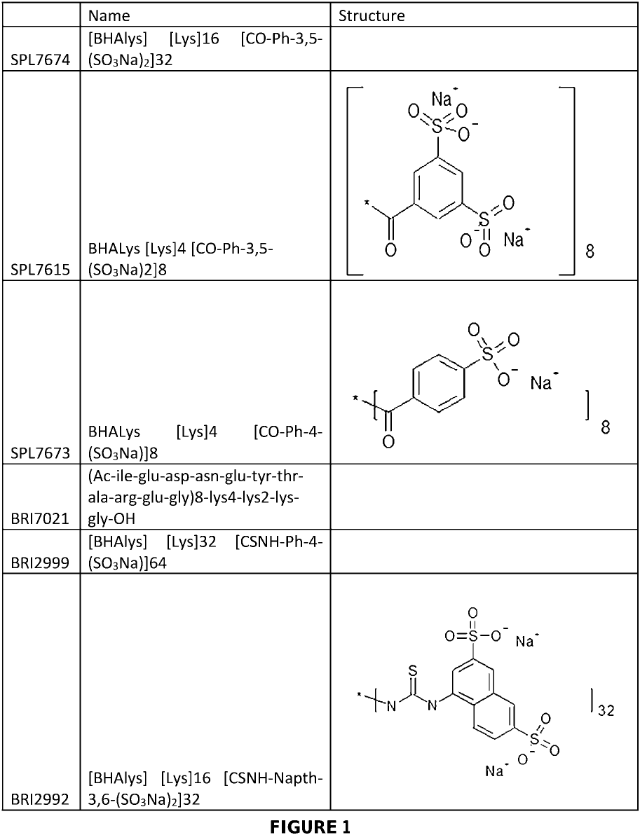

In some embodiments, the macromolecule is not SPL-7674, SPL-7615, SPL-7673, BAI-7021, BRI-2999, and BRI-2992. The structures of these molecules are shown in FIG. 1.

### Coronavirus

As used herein, “Coronaviridae”, known by the common name of “Coronavirus” or “CoV” are enveloped, positive sense, single-stranded RNA viruses. There are two subfamilies of Coronaviridae, Letovirinae and Orthocoronavirinae. The phylogeny of coronaviruses is outlined in Coronaviridae Study Group (2020).

In one embodiment, the CoV is selected from the genera Alphacoronavirus (alphaCoV), Betacoronavirus (betaCoV), Gammacoronavirus (gammaCoV) and Deltacoronavirus (deltaCoV).

In one embodiment, the alphaCoV is selected from coronavirus 229E (HCoV-229E), human coronavirus NL63 (HCoV-NL63), transmissible gastroenteritis virus (TGEV), porcine epidemic diarrhea virus (PEDV), and feline infectious peritonitis virus (FIPV).

In one embodiment, the betaCoV is selected from human coronavirus HKU1 (HCoV-HKU1), Human coronavirus OC43 (HCoV-OC43), Severe acute respiratory syndrome-related coronavirus (SARS-CoV), Severe acute respiratory syndrome-related coronavirus-2 (SARS-Cov-2), Middle-East respiratory syndrome-related coronavirus (MERS-CoV), murine hepatitis virus (MHV) and/or bovine coronavirus (BCoV).

In one embodiment, the CoV is capable of infecting a human.

In one embodiment, the CoV capable of infecting a human is selected from: SARS-CoV-2, HCoV-OC43, HCoV-HKU1, HCoV-229E, HCoV-NL63, SARS-CoV, and MERS-CoV or a subtype of variant thereof.

In one embodiment, the CoV has a death rate in humans of about 0.001 to about 10%. In one embodiment, the CoV has a death rate in humans of about 0.01 to about 9%. In one embodiment, the CoV has a death rate in humans of about 0.01 to about 9%. In one embodiment, the CoV has a death rate in humans of about 0.01 to about 7%. In one embodiment, the CoV has a death rate in humans of about 0.01 to about 6%.

In one embodiment, the CoV has a median daily time-varying basic reproduction number (Rt) in humans of about 1.3 to about 5 when minimal social restrictions are in place. In one embodiment, the CoV has an Rt in humans of about 1.4 to about 4 when minimal social restrictions are in place. In one embodiment, the CoV has an Rt in humans of about 1.4 to about 3 when minimal social restrictions are in place. In one embodiment, the CoV has an Rt in humans of about 1.4 to about 2.6 when minimal social restrictions are in place. In an embodiment, the Rt is calculated as described in Kucharski et al 2020.

In one embodiment, the CoV is SARS-CoV-2 or a subtype or variant thereof. In one embodiment, the SARS-CoV-2 is SARS-CoV-2 subtype L as described in Tang et al., 2020. In one embodiment, the SARS-CoV-2 is SARS-CoV-2 subtype S as described in Tang et al., 2020. In an embodiment, SARS-CoV-2 is SARS-CoV-2 hCoV-19/Australia/VIC01/2020 or a variant thereof. In one embodiment, SARS-COV-2 comprises the sequences as described in NCBI Reference Sequence: NC_045512.2 or a variant thereof. In one embodiment, SARS-CoV-2 comprises the sequence as described in GenBank: MN908947.3 or a variant thereof. In an embodiment, the SARS-CoV-2 is B.1.1.7 (also known as known as 20I/501Y.V1 or VOC 202012/01) or a variant thereof. In an embodiment, the SARS-CoV-2 is B.1.351 (also known as 20H/501Y.V2) or a variant thereof. In an embodiment, the SARS-CoV-2 is P1 (also known as 20J/501Y.V3) or a variant thereof. In an embodiment, the SARS-CoV-2 is B.1.526 or a variant thereof. In an embodiment, the SARS-CoV-2 is B.1.427 or a variant thereof. In an embodiment, the SARS-CoV-2 is B.1.429 or a variant thereof.

The B.1.1.7, B.1.351, P.1, B.1.427, and B.1.429 variants are classified as variants of concern by CDC.

Examples of SARS-CoV-2 variants are described, for example, in Shen et al., 2020 and Tang et al., 2020. Foster et al (2020) have found 3 variants, A, B and C, based on genomic analysis. In some embodiments, the SARS-CoV-2 is SARS-CoV-2 variant A. In some embodiments, the SARS-CoV-2 is SARS-CoV-2 variant B. In some embodiments, the SARS-CoV-2 is SARS-CoV-2 variant C.

In one embodiment, the variant is at least 90% identical to the parental sequence. In one embodiment, the variant is at least 92% identical to the parental sequence. In one embodiment, the variant is at least 93% identical to the parental sequence. In one embodiment, the variant is at least 94% identical to the parental sequence. In one embodiment, the variant is at least 95% identical to the parental sequence. In one embodiment, the variant is at least 96% identical to the parental sequence. In one embodiment, the variant is at least 97% identical to the parental sequence. In one embodiment, the variant is at least 98% identical to the parental sequence. In one embodiment, the variant is at least 99% identical to the parental sequence. In some embodiments, the parental strain is SARS-CoV-2 hCoV-19/Australia/VIC01/2020. In some embodiment, the parental strain is BetaCoV/Wuhan/WIV04/2019. In some embodiments, the parental strain is SARS-CoV-2 Slovakia/SK-BMC5/2020. In some embodiments, the parental strain is SARS-CoV-2 2019-nCoV/USA-WA1/2020. In an embodiment, the parental strain is B.1.1.7. In an embodiment, the parental strain is B.1.351. In an embodiment, the parental strain is P1.

CoV infections cause can cause respiratory, enteric, hepatic, and neurological diseases in different animal species, including camels, cattle, cats, and bats.

CoV can be transmitted from one individual to another through contact of viral droplets with mucosa. Typically, viral droplets are airborne and inhaled via the respiratory tract including the nasal airway. Typically, the individual is a human individual. In some embodiments, the individual is a live stock or domestic animal. Typically, during an infection, CoV can be found in the upper respiratory tract, for example the nasal passages. In some examples, CoV can be found in the lower respiratory tract, for example the bronchi and/or alveoli.

In an embodiment, a CoV infection can cause one or more symptoms selected from one or more of: fever, cough, sore throat, shortness of breath, viral shedding respiratory insufficiency, runny nose, nasal congestion, malaise, bronchitis, headache, muscle pain, dyspnea, moderate pneumonia, severe pneumonia, acute respiratory distress syndrome (ARDS). In an embodiment, the ARDS is selected from mild ARDS (defined as 200 mmHg < PaO2/FiO2 ≤ 300 mmHg), moderate ARDS (defined as 100 mmHg < PaO2/FiO2 ≤ 200 mmHg) and severe ARDS (defined as PaO2/FiO2 ≤ 100 mmHg).

In an embodiment, a SARS-CoV-2 infection can cause one or more symptoms selected from one or more of: fever, cough, sore throat, shortness of breath, viral shedding, respiratory insufficiency, runny nose, nasal congestion, malaise, bronchitis, headache, muscle pain, dyspnea, moderate pneumonia, severe pneumonia, acute respiratory distress syndrome (ARDS).

In an embodiment, the macromolecule reduces the NEWS (National Early Warning Score) or NEWS2 score of the individual. In another embodiment, the macromolecule or pharmaceutically acceptable salt thereof reduces the viral load of the individual. A person skilled in the art will appreciate that viral load can be measured by any method known to a person skilled in the art including for example, measurement by Quantitative reverse transcription PCR (RT-qPCR) to the relevant viral nucleotide sequences. In one embodiment, viral load is reduced to above 20CT (cycle threshold), or reduced to above 30CT, or reduced to above 35CT, or reduced to above 40CT.

In one embodiment, the macromolecule reduces the CoV antibody titre of the individual. In one embodiment, the IgA, IgG and/or IgM antibody titre is measured by ELISA, and is reduced to below detectable levels. In some embodiments, the antibody is to protein S or N. In some embodiments, the sample tested is taken from oral swabs, nasal swabs, blood sample, throat swabs or lung fluid.

In some embodiments, the macromolecule is retained within the lung and does not leach into the systemic circulation. In some embodiments, the percentage of macromolecule that reaches the systemic circulation is less than 10%, less than 25%, less than 50% and less than 70%. Systemic delivery refers to the delivery of the drug pharmaceutically active agent to the blood from the lungs, either directly via absorption into lung capillaries or after absorption into pulmonary lymphatic capillaries.

In an embodiment, the CoV is not SARS-CoV. In one embodiment, the CoV is not an alphaCOV. In one embodiment, the CoV is not a canine coronavirus.

### Respiratory Syncytial Virus

As used herein, “Orthopneumovirus”, known by the common name of “Respiratory syncytial virus” or “RSV” are negative-sense, single-stranded RNA viruses. RSV is a member of the Pneumoviridae family. RSV primarily infects respiratory epithelial cells. There is a single RSV serotype with two major antigenic subgroups, A and B as outlined in Borchers et al (2013). The subtypes can be determined based on the reactivity of the F and G surface proteins to monoclonal antibodies. RSV infections cause can cause symptoms in primates, humans, rats, mice, cows, guinea pigs, ferrets, and hamsters.

In one embodiment, the RSV is a human RSV (HRSV). In one embodiment, the HRSV is HRSV long.

In one embodiment, the RSV is selected from RSV subtype A (RSVA) or RSV subtype B (RSVB).

In an embodiment, the RSVA is selected from GA1, GA2, GA3, GA4, GA5, GA6, and GA7 clades as described in Melero et al (2013). In an embodiment, the RSVA is selected from GA2 and GA5. In an embodiment, GA2 clade includes NA1, NA2, CB-A, and ON1 genotypes. In an embodiment, the RSVB is one or more of GB1, GB2, GB3/SAB3, GB4, and BA. In an embodiment, the RSVB is BA clade.

In an embodiment, the RSVA is a member of one of the twenty-three genotypes identified in Ramaekers et al 2020. In an embodiment, the RSVA is selected from genotype A1, A2, A3, A4, A5, A6, A7, A8, A9, A10, A11, A12, A13, A14, A15, A16, A17, A18, A19, A20, A21, A22 and A23. In an embodiment, the RSVB is a member of one of the six RSVB genotypes identified in Ramaekers et al 2020. In an embodiment, the RSVB is selected from genotype B1, B2, B3, B4, B5 and B6.

In an embodiment, the RSV infection causes one or more of the following symptoms: congested or runny nose, decrease in appetite, coughing, mucus when coughing (yellow, green, or gray mucus), sneezing, sore throat, mild headache, fever, wheezing, rapid breathing or difficulty breathing, bluish colour of the skin (cyanosis), severe asthma symptoms in individuals with asthma, acute bronchitis, severe bronchitis, airway inflammation, airway congestion, chronic obstructive pulmonary disease, heart congestion, bacteremia, pneumonia, acute otitis media, and recurrent otitis media.

RSV infections can result in secondary infections such as for example bacteremia, pneumonia, acute otitis media, and recurrent otitis media.

RSV can be transmitted from one individual to another through contact of viral droplets with mucosa. Typically, viral droplets are airborne and inhaled via the respiratory tract including the nasal airway. Typically, the individual is a human individual. In some embodiments, the individual is a live stock or domestic animal. In an embodiment, the livestock is a cow. Typically, during an infection, RSV can be found in the upper respiratory tract, for example the nasal passages. In some examples, RSV can be found in the lower respiratory tract, for example the bronchi and/or alveoli.

In an embodiment, the macromolecule or pharmaceutically acceptable salt thereof reduces the viral load of the individual. A person skilled in the art will appreciate that viral load can be measured by any method known to a person skilled in the art including for example, measurement by Quantitative reverse transcription PCR (RT-qPCR) to the relevant viral nucleotide sequences. In one embodiment, viral load is reduced to above 20CT (cycle threshold), or reduced to above 30CT, or reduced to above 35CT, or reduced to above 40CT.

In one embodiment, the macromolecule reduces the RSV antibody titre of the individual. In one embodiment, the IgA, IgG, IgM and/or IgE antibody titre is measured by ELISA, and is reduced to below detectable levels. In some embodiments, the sample tested is taken from oral swabs, nasal swabs, blood sample, throat swabs or lung fluid.

In some embodiments, the macromolecule is retained within the lung and does not leach into the systemic circulation.

In some embodiments, the percentage of macromolecule that reaches the systemic circulation is less than 10%, less than 25%, less than 50% and less than 70%. Systemic delivery refers to the delivery of the drug pharmaceutically active agent to the blood from the lungs, either directly via absorption into lung capillaries or after absorption into pulmonary lymphatic capillaries.

Treatment of RSV can include one or more of the following: hospitalisation, intensive care treatment, ICU admission, intubation and supplemental oxygen.

### Methods and Uses

The present invention relates to methods and uses for preventing or reducing the likelihood of CoV and/or a RSV infection in an individual, preventing or reducing the likelihood of a symptom associated with a CoV and/or a RSV infection in an individual, reducing the severity and/or duration of a CoV and/or a RSV infection in an individual, treating a CoV and/or a RSV infection in an individual, preventing or reducing viral shedding in an individual infected with a CoV and/or aRSV infection, or reducing transmission of a CoV and/or a RSV in a population, comprising administering to the individual an effective amount of a macromolecule as described herein.

In one embodiment, the macromolecule as described herein is intended for administration to the respiratory tract. As used herein, the term “respiratory tract” refers to the passage formed by the mouth, nose, throat, and lungs, through which air passes during breathing. A reference to the respiratory tract includes both the upper respiratory tract and/or lower respiratory tract. In one embodiment, the macromolecule as described herein is intended for administration to the upper respiratory tract. In one embodiment, the macromolecule as described herein is intended for administration to the lower respiratory tract. A person skilled in the art will appreciate that the upper respiratory tract comprises one or more of the: nasal cavity, oral cavity, sinuses, throat, pharynx and larynx. A person skilled in the art will appreciate that the nasal cavity comprises one or more of the: vestibular area, olfactory area, superior turbinate, middle turbinate, inferior turbinate and the nasopharynx. A person skilled in the art will appreciate that the lower respiratory tract comprises one or more of the: trachea, primary bronchi and lungs. In some embodiments, the macromolecule is delivered nasally. In an embodiment, administering a macromolecule comprises administering to the mucosa of one or more areas of the respiratory tract. In an embodiment, the macromolecule is administered to the nasal cavity. In an embodiment, the macromolecule as described herein is administered to the nasal mucosa. In an embodiment, the macromolecule is administered to one or more of the nasal turbinates, nasopharynx, and/or oropharynx. In an embodiment, the macromolecule as described herein is administered to the oral mucosa. In an embodiment, the macromolecule as described herein is administered to the mucosa of the primary bronchi. In an embodiment, the macromolecule as described herein is administered to the mucosa of the lungs.

The lung is known to be a particularly harsh environment for stability of active agents. Small molecules quickly pass through the lung epithelium and are cleared into the vascular system. Particle size is important to reach the relevant diseased structures within the lung. Another difficulty encountered in delivery of large particles to the lung is that the action of cilia in the lung will tend to quickly remove agents delivered to the lung, and result in excretion via the faeces. Thus, a particular advantage of some embodiments, of the present invention is that the dendrimer is not degraded or rapidly expelled by the cilia after administration to the lung environment. In some embodiments, the macromolecule is retained within the lung for an extended period of time. In some embodiments, the macromolecule is retained within the lung for up to a month, a week or a day.

In some embodiments, the macromolecule is administered topically. In an embodiment the macromolecule is administered topically to the epidermis or the eye. In some embodiments, topical administration does not cover administration to the respiratory tract.

In some embodiments, the macromolecule is administered dermally. For example, the macromolecule may be administered dermally to one or more of a hand, wrist, forearm, face and neck.

In some embodiments, the macromolecule is delivered via a parenteral routes (e.g. intravenous, subcutaneous or intramuscular) for systemic delivery. In some embodiments, the macromolecule is delivered by bolus or infusion. In some embodiments, the macromolecule is delivered via injection. In some embodiment, the macromolecule is delivered intravenously.

In some embodiments, the macromolecule is applied to a surface. In some embodiments, the macromolecule is applied to surfaces, including metal, polymers such as paint, plastic and rubber, fabric, polymers, wood, ceramics, glass, concrete, skin, human tissues, mucosa and bone. In some embodiments, the macromolecule is applied to personal protective equipment (PPE), including gloves, masks, gowns and scrubs. In some embodiments, the macromolecule is applied to wipes and tissues. In some embodiments, the macromolecule is applied to the surgical/medical field including the patient, the table, and equipment. The surgical/medical field may be for human or veterinary use.

### Compositions

In some embodiments, a composition comprising the macromolecule and a pharmaceutically acceptable carrier is used. The compositions as described herein are suitable for e.g. respiratory administration via nasal, pulmonary, ocular administration, dermal administration and/or parenteral administration.

The pharmaceutical composition may also include polymeric excipients/additives or carriers, e.g., polyvinylpyrrolidones, derivatized celluloses such as hydroxymethylcellulose, hydroxyethylcellulose, and hydroxypropylmethylcellulose, microcrystalline cellulose /carboxy methyl cellulose, Ficolls (a polymeric sugar), hydroxyethylstarch (HES), dextrates (e.g., cyclodextrins, such as 2-hydroxypropy1-β-cyclodextrin and sulfbbutylether-β-cyclodextrin), dextrans, PVP, inulin, polyethylene glycols, and pectin. The pharmaceutical composition may also include amino acid or sugar carriers, e.g., glycine, leucine, alanine, mannitol and trehalose. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19.sup.th ed., Williams & Williams, (1995), and in the “Physician’s Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The carrier, excipient or diluent may include one or more of any and all conventional solvents, dispersion media, fillers, solid carriers, aqueous solutions, coatings, viscosity modifying agents, isotonic agents, and absorption enhancing or delaying agents, activity enhancing or delaying agents and the like. The use of such media and agents for pharmaceutically active substances is well known in the art, and it is described, by way of example, in Remington’s Pharmaceutical Sciences, 18th Edition, Mack Publishing Company, Pennsylvania, USA. Except insofar as any conventional carrier and/or diluent is incompatible with the active ingredient, use thereof in the compositions of the present invention is contemplated.

In some embodiments, the composition of macromolecule comprises a rheology modifying agent, especially a polyacrylic acid (carbomer), for example, Carbopol® polymer such as Carbopol® 971P, 974P or 71G or Noveon Polycarbophil, from Lubrizol or their equivalent. In some embodiment, the rheology modifying agent is Carbopo1®974P. They may be homopolymers of acrylic acid, or crosslinked with an allyl ether of pentaerythritol, allyl ether of sucrose, or allyl ether of propylene. In an embodiment, it is a carbomer. In an embodiment, it is a carboxypolymethylene. In an embodiment, it is an acrylic acid polymer. It will be appreciated by a person skilled in the art that chains can have different lengths, different degrees of cross-linking, molecular weight and the like and can be of different grades for specific uses (such as pharmaceutical which is designated by a P). In some embodiments the Carbopol polymer is a NF (national formulatory) version. A person skilled the art will be aware of when it is suitable to use pharmaceutical and non-pharmaceutical grades. The rheology modifier may be present in an amount of 1-10% w/w, especially about 2 to 5% w/w, or 0.01 to 0.1% w/w. In some embodiments, the rheology modifier is carbopol. The carbopol rheology modifier is present in an amount such as 0.01% to 1% w/w, or about 0.01 to 0.1%, especially 0.05% to 0.1%, especially 0.05% w/w. In some embodiments, the carbopol is Carbopol 974. In some embodiment, Carbopol 974 is present in an amount such as 0.05% w/w to about 5% w/w, or about 0.05% w/w to about 3% w/w, or about 0.05% w/w to about 2% w/w, or about 0.05% w/w to about 1% w/w, or about 1%, or about 0.05% w/w Carbopol 974. In some embodiments, the carbopol is Carbopol 971. In some embodiment, Carbopol 971 is present in an amount such as 0.05% w/w to about 1% w/w, or about 0.05% w/w to about 1.5% w/w, or about 0.05% w/w to about 1.8% Carbopol 971. In some embodiments, the rheology modifying agent is a cellulose, for example, hydroxypropylmethyl-cellulose or microcrystalline cellulose /carboxy methyl cellulose. In some embodiment, the rheology modifying agent is hydroxypropylmethyl-cellulose. In some embodiments, hydroxypropylmethyl-cellulose is present in an amount, such as 0.01% to 1% w/w, or about 0.05 to 0.5% w/w, especially about 0.1%. In some embodiment, the rheology modifying agent is microcrystalline cellulose /carboxy methyl cellulose. In some embodiments, microcrystalline cellulose /carboxy methyl cellulose is present in an amount, such as 0.5% to 5% w/w, or about 1% to 3% w/w, especially about 2% w/w. The rheology modifier aids the composition in having bioadhesive/mucoadhesive properties.

The composition of macromolecule may also include a chelating agent, such as a polyaminocarboxylic acid. A particularly useful chelating agent is ethylenediamine tetraacetic acid (EDTA) and its salts. Suitable amounts of chelating agent are in the range of 0.001% to 2% w/w, especially 0.005% to 1% w/w. In some embodiments, the chelating agent is present in a low amount, such as 0.001% to 0.1% w/w, especially about 0.005%. Other ingredients that may be included in the gel composition include preservatives such as parabens in an amount of up to 1% w/w, for example methylparaben and propylparaben or mixtures thereof. Suitable amounts of parabens are in the range of 0.01% to 0.5% w/w, especially 0.01% to 0.2% w/w. In some embodiments, methyl paraben is present in an amount, such as 0.05% to 0.2% w/w, especially about 0.18%. In some embodiments, methyl paraben is present in an amount, such as 0.14% to 0.23% w/w. In some embodiments, propyl paraben is present in an amount, such as 0.01% to 0.05% w/w, especially about 0.02%. In some embodiments, propyl paraben is present in an amount, such as 0.015% to 0.0025% w/w. In some embodiments, benzalkonium chloride is present in an amount, in the range of 0.01% to 0.1%w/w, especially about 0.05%.

Other ingredients that may be included in the composition include, for example, solvents such as water, pH adjusting agents such as hydroxide and or hydrocholic acid, and emollients and humectants such as glycerin and propylene glycol, in an amount of up to 5%. In some embodiment, glycerin (glycerol) is present. In some embodiments, glycerin is present in an amount, such as 0.1% to 5% w/w, 0.5 % to 2% w/w, especially about 1% w/w. In some embodiments, propylene glycol is present. In some embodiments, propylene glycol is present in an amount, such as 0.1% to 5%w/w, 0.5 % to 2%w/w, especially about 1%w/w.

In an embodiment, the composition, when delivered by a nasal spray device as described herein, creates a moisturizing and protective barrier in the nasal cavity. In an embodiment, the composition, when delivered by a nasal spray device as described herein, creates a moisturizing and protective barrier on the nasal mucosa.

### Respiratory Compositions (Nasal and Oral)

In some embodiments, administering the macromolecule to the respiratory tract may include delivering the macromolecule to the diseased lung by an oral or nasal route; to the upper respiratory tract via the nasal route; or to the nasal cavity and/or the nasal mucosa via the nasal route. For example, in some embodiments, the macromolecule may be delivered by inhalation, such as inhalation via the mouth and/or nose. In some embodiments, the macromolecule may be delivered by intratracheal instillation or insufflation. As such, the macromolecule may be delivered to the respiratory tract without the need for a separate targeting agent that targets the pharmaceutically active agent to the diseased tissue or cells.

For example, in some embodiments, pharmaceutical compositions may be aerosol compositions, nebulized compositions, dry powder compositions, aqueous compositions or insufflation compositions. In some embodiments, pharmaceutical compositions may be included in pressurized metered dose inhalers, dry powder inhalers, nebulizers, sprays and the like. In an embodiment, the composition is suitable for administration in a nasal spray, an oral spray, an inhaler or a nebuliser. For additional discussion, see Zarogoulidis et al (2012).

In some embodiments, the macromolecule is formulated for nasal delivery. In some embodiments, the macromolecule is formulated for delivery to the nasal cavity. In some embodiments, the macromolecule is formulated for delivery to the nasal mucosa. In some embodiment, the composition is formulated for delivery to one or more of nasal turbinates, nasopharynx, and/or oropharynx.

In some embodiments, the pharmaceutical composition may be suitable for intra nasal delivery, such as an aqueous nasal spray composition or a dry powder nasal spray. Nasal spray compositions may include purified aqueous solutions of the active agent with preservative agents and isotonic agents. Such compositions may be adjusted to a pH and isotonic state compatible with the nasal mucous membranes. In some embodiments, the macromolecule is delivered as a powder, a gel, a liquid, an aerosol or an emulsion. In some embodiments, the pH of the composition is about 4.5 to about 7.42. In some embodiments, the pH of the composition is about 5 to about 7. In some embodiments, the pH of the composition is about 5 to about 6.5. In some embodiments, the pH is about 5.5 to about 6.5. In other embodiments, the pH is about 7.4.

In some embodiments, the osmolality of the composition is about 200 to about 700 Osmol/kg. In some embodiments, the osmolality of the composition is about 300 to about 600 Osmol/kg. In some embodiments, the osmolality of the composition is about 300 to about 700 Osmol/kg. In some embodiments, the osmolality of the composition is about 200 to about 400 Osmol/kg, more preferably about 280 Osmol/Kg. Osmolality regulators include NaCl, lysine, CaCl₂, sodium citrate and pH regulators include H₂SO₄, NaOH, tromethamine, HCl. In some embodiments, the osmolality of the composition is about 200 to about 400mOsmol.

In some embodiments, the composition comprises methylparaben at about 0.14% to about 0.23%.

In some embodiment, the composition comprises propylparaben at a concentration of about 0.015% to about 0.025%.

In an embodiment, the nasal spray composition has antiviral activity against CoVs. In an embodiment, the nasal spray composition inactivates more than 90%, or more than 92%, or more than 95%, or more than 99%, or more than 99.9% of CoV. In an embodiment, the nasal spray composition inactivates more than 90%, or more than 92%, or more than 95%, or more than 99%, or more than 99.9% of SARS-CoV-2. In an embodiment, the nasal spray composition inactivates more than 90%, or more than 92%, or more than 95%, or more than 99%, or more than 99.9% of CoV that cause COVID-19. In an embodiment, the nasal spray composition has antiviral activity against a RSV virus. In an embodiment, the nasal spray composition inactivates more than 90%, or more than 92%, or more than 95%, or more than 99%, or more than 99.9% of RSV. In an embodiment, inactivation is after at least 1 minute of exposure to a composition as described herein. In an embodiment, the nasal spray composition provides a moisture layer to help keep nasal tissue hydrated. Hydration of the nasal tissue protects it from dryness and damage making it more difficult for viral penetration.

In some embodiment, the macromolecule is formulated for delivery to the lung. Neutral pH and tonicity are important factors for lower respiratory delivery to avoid bronchoconstriction in patients with respiratory impairment, as the lungs are poorly buffered.

In some embodiments, the pharmaceutical composition may be a dry powder with particle sizes greater than 0.5 µm and less than 50 µm. In some embodiments, the particle size is less than 5 um, greater than 1 um.

In some embodiments, the macromolecule may have a particulate size of less than about 100 nm. In other embodiments, macromolecule may have a particulate size between about 1 and about 10 nm, between about 2 and about 8 nm, and between about 3 and about 6 nm by DLS. In some embodiments, the macromolecules may have a mean size of about 5 nm by DLS (at 1 mg/ml in 10-2 M NaCl). In some embodiments, the macromolecule may have a molecular weight of less than 30 kDa, between about 10 to about 30 kDa, and between about 10 to about 20 kDa.

Examples of ingredients suitable for nasal or oral delivery include are provided below in Table 1.

TABLE 1

Suitable ingredients for nasal delivery

| | | |
|---|---|---|
| Ingredients | IIG for nasal route, %w/w | Function |
| Alcohol (ethanol), 200 proof | 2 | Co-solvent |
| Anhydrous dextrose | 0.5 | tonicity |
| Anhydrous trisodiumcitrate | 0.0006 | buffer |
| Benzyl alcohol | 0.0366 | preservative |
| Benzalkonium chloride | 0.119 | preservative |
| Butylated hydroxyanisole | 0.0002 | antioxidant |
| Cellulose microcrystalline | 2 | Suspending agent, stabilizer |
| Chlorobutanol | 0.5 | preservative |
| Carboxymethyl cellulose Na | 0.15 | Suspending agent |
| Hydroxypropyl methylcellulose | (4- topical) | |
| Edetate disodium | 0.5 | Chelator, antioxidant |
| Hydrochloric acid | Not reported | pH adjustment |
| Methylparaben | 0.7 | preservative |
| Oleic acid | 0.132 | Penetration enhancer |
| PEG400 | 20 | Surfactant, co-solvent |
| PEG3500 | 1.5 | surfactant |
| Phenylethyl alcohol | 0.254 | Preservative, masking agent |
| Polyoxyl 400 stearate | 15 | surfactant |
| Polysorbate 20 | 2.5 | surfactant |
| Polysorbate 80 | 10 | surfactant |
| Propylene glycol | 20 | Co-solvent |
| Propylparaben | 0.3 | Preservative |
| Sodium chloride | 1.9 | tonicity |
| Sodium hydroxide | 0.004 | pH adjustment |
| Sulfuric acid | 0.4 | pH adjustment |
| Succinic Acid | | |
| Disodium Succinate | | |
| Zinc Acetate | | |
| Sugars, or flavouring agents e.g. Sodium Saccharin | | |

The rapid mucociliary clearance in the nasal cavity and presence of nasal lysozymes and macrophage can present challenges to mucosal delivery. Mucoadhesive excipients may be required. Depending on the intended mode of administration, the compositions may comprise a bioadhesive agent. In an embodiment, the bioadhesive is a mucoadhesive polymer. A bioadhesive agent may alter the viscosity, rheology and/or the ciliary beating frequency (CBF). Examples of mucoadhesive polymers include poly(acrylates), chitosan, cellulose and derivatives including carboxymethylcellulose and hydroxypropyl cellulose, hyaluronic acid derivatives, pectin, traganth, starch, poly(ethylene glycol), sulfated polysaccharides, carrageenan, sodium alginate, polyvinyl alcohol, polyvinylpyrrolidone, acacia gum, alginic acid, and gelatine. In an embodiment, the composition may comprise a nasal mucoadhesive component.

However, viscosity should not impede airflow. In some embodiments, viscosity of the composition is between 1 and 10000 cP, or between 1 and 1000 cP, or between 100 and 1000 cP, or between 100 and 500 cP, or between 100 and 400 cP, or between 150 and 300 cP, or between 150 and 250 cP, or between 1 and 200 cP, or between 1 and 100 cP, or between 1 and 50 cP, or between 1 and 25 cP, or between 1 and 10 cP. In a preferred embodiment, the viscosity of the composition is about 1 to about 10 cP (in comparison, SPL7013 gel for vaginal use has a viscosity of 20,000 to 60,000 cP). In some embodiments, the kinematic viscosity of the solution is below 1000, or below 500 mm²s⁻¹.

For lung delivery, viscosity should be low. In some embodiments, the viscosity is less than 200 cP. In some embodiments, the viscosity is less than 100 cP.

For nasal delivery, viscosity should be low. In some embodiments, the viscosity is less than 100 cP. In some embodiments, the viscosity is less than 50 cP. In some embodiments, the viscosity is less than 20 cP. In some embodiments, the viscosity is less than 15 cP. In some embodiments, the viscosity is less than 10 cP.

In one embodiment, the nasal composition comprises the formulation as shown in Table 2.

TABLE 2

Provides an example of the nasal or oral composition as described herein comprising SPL7013

| | |
|---|---|
| Component | % w/w |
| SPL7013 | 0.5 to 5.0 |
| Purified Water | qs to 100 |
| Glycerin | 0 to 1.0 |
| Propylene Glycol | 0 to 2.0 |
| Methylparaben | 0 to 0.5 |
| Propylparaben | 0 to 0.05 |
| Disodium EDTA | 0.005 to 0.1 |
| Citric acid | 0 to 5.0 |
| Carbomer 971P or 934P, 974P or other viscosity/rheology modifier, e.g. cellulose | 0.1 to 5.0 and 0.05 to 0.1 |
| Benzalkonium chloride | 0 to 0.1 |
| Sodium Hydroxide or other pH modifier | qs to pH 4.5 to 7 |

In some embodiments, the pharmaceutical composition may also include any other therapeutic ingredients, surfactants, propellants, stabilizers, or the like. The carrier(s) must be pharmaceutically acceptable in the sense of being compatible with the other ingredients of the composition and not unduly deleterious to the recipient thereof.

In some embodiments, the pharmaceutical composition may produce particle sizes greater than 0.5 µm and less than 50 µm. In some embodiments, the particle size is less than 5 µm, less than 1 µm, or less than 10 µm.

In one embodiment, the mean particle size is from about 0.21 to about -200 µm. In one embodiment, the mean particle size is from about 1 to about 200 µm. In one embodiment, the mean particle size is from about 1 to about 50 µm. In one embodiment, the mean particle size is from about 1 to about 20 µm. In one embodiment, the mean particle size is from about 1 to about 5 µm.

In some embodiments, a particle diameter of 1 to about 5 µm is good for delivery to the lower airway; from 5 to 10 µm particles deposit mostly in the trachea and bronchi, while diameter > 10 µm particles deposit mostly in the nose. Usually particles less than 10 µm median aerodynamic diameter, can reach the lower airways during nasal breathing. The composition may be a liquid, gel or powder.

In some embodiments, suitable for lower airway delivery the Dv90 is about 5 to 20 µm. In some embodiments, suitable for lower airway delivery the Dv50 is about 5 to 10 µm. In some embodiments, suitable for lower airway delivery the Dv10 is about 1 to 5 µm. In some embodiments, suitable for nasal delivery the Dv10 is greater than about 10, 15 or 20 µm.

In some embodiments suitable for nasal delivery, the Dv50 is greater than about 20, 40 or 60 µm. In some embodiments suitable for nasal delivery, the Dv90 is greater than about 60, 80 or 1000 µm.

In some embodiments suitable for nasal delivery, about 10% to about 0.5% of the particles are about 10 µm or less. In some embodiments suitable for nasal delivery, about 10% to about 0.5% of the particles are about 10 µm or less. In some embodiments suitable for nasal delivery, about 7% to about 0.5% of the particles are about 10 µm or less. In some embodiments suitable for nasal delivery, about 5% to about 0.5% of the particles are about 10 µm or less. In some embodiments suitable for nasal delivery, about 10% to about 0.5% of the particles are about 5 µm or less. In some embodiments suitable for nasal delivery, about 7% to about 0.5% of the particles are about 5 µm or less. In some embodiments suitable for nasal delivery, about 6% to about 0.5% of the particles are about 5 µm or less. In some embodiments suitable for nasal delivery, about 5% to about 0.5% of the particles are about 5 µm or less. In some embodiments suitable for nasal delivery, less than about 10% of the particles are about 6 µm or less. In some embodiments suitable for nasal delivery, less than about 10% of the particles are about 5 µm or less. In some embodiments suitable for nasal delivery, less than about 5% of the particles are about 5 µm or less. In some embodiments suitable for nasal delivery, less than about 5% of the particles are about 5 µm or less.

### Ocular Compositions

The macromolecule of the invention may be delivered in any composition suitable for application to the eye, for example, solutions, ointments, gels, lotions, in slow release polymers or coated, on bound to or impregnated in contact lenses. In an embodiment, the composition can be delivered to the eye in eye drops. In an embodiment, the composition can be delivered to the eye in a spray

By “suitable for application to the eye” is meant that any component of the composition does not cause a long-lasting deleterious effect on the eye or the subject being treated. Transient effects such as minor irritation or “stinging” upon administration may occur without long-lasting deleterious effect. The macromolecule may be formulated as a simple aqueous solution. Alternatively, the macromolecule may be formulated to have one or more of physiologically compatible osmolality and pH, for example, by including salts and buffering agents, and other components such as preservatives, gelling agents, viscosity control agents, ophthalmic lubricating agents, mucoadhesive polymers, surfactants, antioxidants and the like in a solution, gel, lotion or ointment.

The macromolecules of the present invention are retained on or in the epithelium for a period of time, allowing the macromolecule to diffuse out of epithelium. Such diffusion provides slow release of drug into the ocular environment, enabling the anti-viral activity of the macromolecule to be delivered over a period and not be quickly washed away by the ocular fluids and physical cleansing. The macromolecule may be released from the epithelium over a period of greater than 10 minutes, more especially over a period greater than 1 hour, and more especially over a period of more than 6 hours.

In some embodiment, the invention provides a composition comprising a macromolecule as described herein and at least one pharmaceutically acceptable carrier that provides a pH and osmolality compatible with the eye.

Suitable ophthalmically acceptable salts that may be used as osmolality agents include salts having sodium, potassium or ammonium cations and chloride, citrate, ascorbate, borate, phosphate, bicarbonate, sulfate, thiosulfate or bisulfite ions. Examples of suitable salts include sodium chloride, potassium chloride, sodium thiosulfate, sodium bisulfate and ammonium sulfate.

Suitable ophthalmically acceptable pH adjusting agents and/or buffering agents include acids such as acetic, boric, citric, lactic, phosphoric and hydrochloric acids, bases such as sodium hydroxide, sodium phosphate, sodium borate, sodium citrate, sodium acetate, sodium lactate and trishydroxymethylaminomethane, and buffers such as citrate-dextrose, sodium bicarbonate and ammonium chloride.

Suitable preservatives include stabilized ammonium compounds such as benzalkonium chloride, cetyhrimethylammonium chloride and cetylpyridinium chloride, mucuric compounds such as phenyl mercuric acetate, imidazolidinyl urea, parabens such as methyl paraben, ethyl parnben, propyl paraben or butyl paraben; phenoxyethanol, chlorophenoxyethanol, phenoxypropanol, chlorobutanol, chlorocresol, phenylethyl alcohol, ethylenediamine tetraacetic acid, sorbic acid and salts thereof.

Suitable gelling agents or viscosity control agents include gelling agents that increase viscosity when they come into contact with lacrimal fluid, for example, lacrimation caused by blinking or tears. Such gelling agents may he used to reduce loss of the macromolecule by lacrimal drainage and allow the macromolecule to have increased residence time and therefore absorption in the eye or epithelial layer of the eyelids. Suitable gelling agents include gellan gum, especially low acetylated gellan gum, alginate gum or chitosan. The viscosity adjusting agent may also include a film-frmning polymer such as an alkylcellulose such as methyl cellulose or ethylcellulose, a hydroxyalkylcellulose such as hydroxyethyl cellulose or hydroxypropyl methylcellulose, hyaluronic acid or its salts, chondroitin sulfate or its salts, polydextrose, cyclodexirin, polydextrin, maltodextrin, dextrin, gelatine, collagen, polygalacturonic acid derivatives such as pectin, natural gums such as xanthan, locust bean, acacia, trngacanth and carageenan, agar, polyvinyl alcohol, polyvinylpyrrolidone, polyethylene glycol, polymers of acrylamide, acrylic acid and polycyano acryiates and polymers of methylmethacrylate and 2-hydroxy-ethyl methacrylate. The viscosity control agent or gelling agent may be present in an amount of 0.1 % to about 6.5%, w/w of the composition, especially about 0.5% to 4.5% w/w of the composition.

Suitable lubricating agents include polyvinyl alcohol, methyl cellulose, hydroxypropyl methylcellulose and polyviny lpytrnlidone.

Suitable mucoadhesive polymers include hydroxypropyl methylcellulose, carboxymethylcellulose, poly(methylmethacrylate), polyacrylamide, polycarbophil, polyethylene oxide, sodium alginate and dextrin.

Suitable ophthalmically acceptable surfactants include non-10mc surfactants such as polyoxyethylene fatty acid glycerides and vegetable oils including polyoxyethylene (60) hydrogenated castor oil, polyoxyethylene alkylethers and alkylphenyl ethers such as octoxynol 10 and octoxynol 40.

Suitable antioxidants include ascorbic acid and sodium metabisulfate.

Ophthalmic ointments may also include one or more of thickeners such as liquid paraffin, yellow soft paraffin, hard paraffin, and/or wool fat.

In an embodiment, the ocular compositions as described herein are suitable for treating and or preventing a CoV infection. In an embodiment, the ocular compositions are described herein are suitable for preventing, reducing or sequestering CoV vial shedding in an individual with a CoV infection.

In an embodiment, the ocular compositions as described herein are suitable for treating and or preventing a RSV infection. In an embodiment, the ocular compositions are described herein are suitable for preventing, reducing or sequestering RSV viral shedding in an individual with a RSV infection.

While it is possible that the composition of the invention may be formulated with carriers, diluents and excipients commonly used in the art as discussed above in topical eye compositions, it is well known that a number of commonly used preservatives have drawbacks when used in topical eye compositions. For example, some preservatives cause eye irritation and if used for long term therapy, they may cause damage to the eye. Furthermore, some preservatives are not effective against some strains of bacteria causing spoilage of the compositions. Parabens are generally regarded as unsuitable for ophthalmic compositions because of their irritant nature. In some cases eye drop compositions are formulated without preservative to reduce irritancy. However, such compositions must he packaged for single use or refrigerated once they are opened.

In some embodiments, the ocular composition as described herein consists of an aqueous solution of the macromolecule together with at least one pharmaceutically acceptable excipient, wherein the at least one excipient provides a pH of 7.0 to 7.6 and osmolality of 240 to 310 mOsm/kg, especially an osmolality that is isotonic with tears. In other embodiments, the composition comprises an aqueous solution of the macromolecule together with at least one pharmaceutically acceptable excipient, wherein the at least one excipient provides a pH of 7.0 to 7.5 and osmolality of 240 to 310 mOsm/kg but preservatives other than the macromolecule is excluded.

### Other Compositions

In an embodiment, the compositions as described herein are suitable for dermal administration and may be formulated in an aqueous, gel or cream composition.

In an embodiment, the compositions as described herein are suitable for use as a surface spray, wash or wipe, including hand wash, surgical field preparation.

In an embodiment, the compositions as described here are imbedded in, or applied, or conjugated to personal protective equipment (PPE), for example a mask, glove or surgical gown or filters for masks.

In some embodiments, the macromolecules as described herein are formulated in a composition suitable for parenteral delivery. For example, for intravenous delivery, the composition may be an aqueous composition, for example ringers, saline, water or dextrose solution, or may be diluted in 0.9% saline or 5% dextrose for use.

In some embodiments, the compositions are formulated as a lozenge or a throat gargle. Lozenge compositions are described for example in Umashankar et al (2016) and Vera et al (2014).

The compositions as described herein may conveniently be presented in unit dosage form and may be prepared by any of the methods well known in the art of pharmacy. Dosage unit form as used herein refers to physically discrete units suited as unitary dosages for the individuals to be treated; each unit containing a predetermined quantity of active ingredient calculated to produce the desired prophylactic or therapeutic effect in association with the required pharmaceutical carrier and/or diluent.

All methods include the step of bringing the macromolecule into association with a carrier that constitutes one or more accessory ingredients. In general, the compositions may be prepared by bringing the macromolecule into association with a liquid carrier to form a solution or a suspension. Such dosage forms are contemplated to be administered as an over a period of time (e.g., for an inhaled dose from about a few seconds to about 2-6 hours, for a parenteral dose, from a few seconds for a bolus, to 24 hours for an infusion).

### Effective Amount

The methods of the present disclosure require administration of an effective amount of macromolecule, or of compositions comprising the macromolecule. An “effective amount” means an amount necessary to at least partially attain the desired response, or to delay the onset of, inhibit the progression of or halt altogether infection. An effective amount for a human patient may, for example, fall within the range of about 0.5 mg to about 5 mg. An effect amount for a human patient may, for example, fall within the range of about 0.5 mg to about 5 mg per actuation per nostril.

In some embodiments, the effective amount is in the range of about 0.04 mg to about 1 g, about 10 mg to about 500 mg, about 10 mg to about 100 mg, or about 100 mg to about 500 mg. In some embodiments, the effective amount is in the range of about 0.5 to 5 mg. In some embodiments, the effective amount is in the range of about 0.5 to 1.5 mg. In some embodiments, the effective amount is about 1 mg. In some embodiments, the effective amount is about 0.5 mg.

In some embodiments, the effective amount is in the range of about 0.1 mg to about 1 g/m², about 1 mg to about 100 mg/m², about 10 mg to about 100 mg/m², or about 10 mg to about 500 g/m².

In some embodiments, the macromolecule is delivered at 0.1 to 10 mg/kg/daily. In another embodiment, the macromolecule is delivered at 1 to 10 mg/kg daily. In another embodiment, the macromolecule is delivered at 0.1 to 1 mg/kg daily.

In some embodiments, the macromolecule is delivered via an infusion of 0.01 to 5 g/day. In another embodiment, the macromolecule is delivered via an infusion of 0.1 to 2 g/day. In some embodiments, the macromolecule is delivered via an infusion of 1 to 2 g/day. In some embodiments, the macromolecule is delivered via an infusion of 0.5 to 1 g/day.

In some embodiments, the composition containing the macromolecule is formulated to contain an amount of macromolecule effective to establish an in vivo concentration of macromolecule in the range of from about 0.050 to about 25 µM. An in vivo concentration is a blood plasma concentration or a lung fluid concentration, or a tissue concentration such as a lung tissue concentration. In some embodiments, the composition containing the macromolecule is formulated to contain an amount of macromolecule effective to establish an in vivo concentration of macromolecule of about 1.0, 2.0, 3.0, 4.0, 5.0, 6.0, 7.0, 8.0, 9.0, 10, 11, 12, 13, 14, or about 15 µM. In some embodiments, the composition containing the macromolecule is formulated to contain an amount of macromolecule effective to establish an in vivo concentration of macromolecule of at least 0.5 µM, at least 0.75 µM, at least 1 µM, or at least 2 µM. Each such value may be combined to form a range with an upper value of about 20 µM, about 17 µM or about 15 µM. In some embodiments, the composition containing the macromolecule is formulated to contain an amount of macromolecule effective to establish an in vivo concentration of macromolecule in the range of from about 0.1 to about 100 µM, from about 0.5 to about 50 µM, or from about 1 to about 25 µM.

In some embodiments, a single dose of 10 to 50 mg/kg would achieve an effective concentration. In another embodiment, a single dose of 20 to 40 mg/kg would achieve an effective concentration. In another embodiment, a single dose of 30 mg/kg would achieve an effective concentration.

Typically, when infused, the macromolecule will be infused at a rate so as to establish and/or maintain an in vivo concentration of macromolecule which is greater than the EC₅₀, preferably greater than the EC₉₀, and which avoids undue side effects.

In some embodiments, the macromolecule is infused at a rate so as to establish and/or maintain an in vivo concentration of macromolecule of at least 0.08 µM, at least 0.9, at least 0.75 µM, at least 1 µM, at least 2 µM, at least 3 µM, at least 4 µM, at least 5 µM, at least 10 µM, or at least 20 µM. In some embodiments, the macromolecule is infused at a rate so as to establish and/or maintain an in vivo concentration of macromolecule of at least 0.001 mg/ml, at least 0.005 mg/ml, at least 0.01 mg/ml, at least 0.02 mg/ml, at least 0.03 mg/ml, at least 0.04 mg/ml, at least 0.05 mg/ml, at least 0.1 mg/ml, at least 0.2 mg/ml, at least 0.3 mg/ml.

In some embodiments, the macromolecule is infused at a rate so as to establish and/or maintain an in vivo concentration of macromolecule in the range of from about 0.01 to about 100 µM, from about 0.5 to about 50 µM, from about 1 to about 50 µM, from about 2 to about 50 µM, from about 5 to about 50 µM or from about 10 to about 50 µM.

In some embodiments, the macromolecule is infused at a rate so as to establish and/or maintain an in vivo concentration of macromolecule in the range of from about 0.001 mg/ml to about 2 mg/ml, from about 0.01 mg/ml to about 1 mg/ml, or from about 0.05 to about 0.5 mg/ml.

In some embodiments, in order to establish and/or maintain the in vivo concentration of macromolecule at a desired concentration, the macromolecule is infused at a desired rate. For example, if targeting a concentration of about 400 mg/L, the infusion rate may in some embodiments be in the range of from about 500 to about 3000 mg/hr, from about 1000 to about 2000 mg/hr, or from about 1500 to about 1600 mg/hr (e.g. 1584 mg/hr) (e.g.400 mg/L × 3.96 L/hr). For example, if targeting a concentration of about 200 mg/L, the infusion rate may in some embodiments be in the range of from about 250 to about 1500 mg/hr, from about 500 to about 1000 mg/hr, or from about 750 to about 800 mg/hr (e.g. 792 mg/hr) (e.g.200 mg/L × 3.96 L/hr).

In some embodiments, the effective amount is formulated at about 0.1% to about 10%w/w, or about 0.5% to about 10%,w/w or about 0.5% to 5%w/w, or about 0.5% to 3%w/w, or about 1% to 3% w/w of macromolecule. In some embodiments, the effective amount is formulated at about 0.5%w/w, at about 1%w/w, at about 2%w/w, at about 3%w/w, at about 4%w/w or about 5% w/w macromolecule. In some embodiments, the composition comprises about 0.5 mg/ml, or about 1 mg/ml, or about 2 mg/ml, or about 2.5 mg/mL, or about 5 mg/ml, or about 10 mg/ml, about 20 mg/ml, about 30 mg/ml macromolecule.

In some embodiments, the composition is administered in a volume of about 0.1 to about 50 ml, about 0.2 ml to about 1 ml, about 1 to about 25 ml, about 0.025 ml to 0.2 ml, or about 5 ml. In some embodiments, the composition is administered in a volume of about 0.025 ml, 0.05 ml, 0.1 ml or about 0.2 ml.

When used in a delivery system the amount of antiviral composition included in the delivery system according to the present disclosure may for example be from about 0.10 g to about 2 g, or from about 0.1 g to about 0.5 g, or from about 0.1 g to about 0.25 g.

In some embodiments, when the composition is for nasal delivery the dose may administered in two actuations (sprays), one in each nostril. In some embodiments, when the composition is for nasal delivery the dose may administered in a volume of about 5 µL to about 200 µL, about 5 µL to about 150 µL, about 5 µL to about 100 µL, 5 µL to about 80 µL, 5 µL to about 70 µL, 5 µL to about 50 µL, 5 µL to about 40 µL, 5 µL to about 30 µL, or about 5 µL to about 10 µL per nostril. In a preferred embodiment, the dose is administered in a volume of about 100 µL per nostril. The macromolecule may be administered on a dosage regimen that provides the desired effect. For example, the dosage, macromolecule, or composition, may be administered from 1 to 8 times per day, from 1 to 6 times per day, from 1 to 5 times per day, from 1 to 4 times per day, from 1 to 3 times per day or once daily. In an embodiment, the dosage, macromolecule or composition may be administered from 1 to 4 times per day. In an embodiment, the macromolecule, or composition is administered to each nostril (e.g. 4 times a day includes 4 times a day in each nostril). In some embodiments, the dosage, composition or macromolecule is administered for about 1 to 2 weeks, about 1 month, about 3 months or about 6 months. In some embodiments, the dosage, composition or macromolecule is administered, once per day, 4 times per day, 6 times per day, or 8 times per day. In an embodiment, the dosage, macromolecule or composition may be administered up to 4 times per day. In an embodiment, the dosage, macromolecule or composition may be administered up to 8 times per day. In some embodiments, the dosage, composition or macromolecule is administered for up to 10 consecutive days. In some embodiments, the dosage, composition or macromolecule is administered for up to 20 consecutive days. In some embodiments, the dosage, composition or macromolecule is administered for up to 30 consecutive days.

### Delivery Devices

In an aspect the present invention provides a device for delivering a nasal, oral or pulmonary composition comprising a macromolecule as described herein. The devices as described herein can deliver the macromolecule to the upper and/or lower respiratory tract. In an embodiment, the device can deliver the macromolecule to the nasal cavity. In an embodiment, the device can deliver one or more doses. In an embodiment, the device is reusable.

In some embodiments, of the device as described herein comprises a composition as described herein.

In an embodiment, the device is a nasal delivery device. In an embodiment, the device is an oral delivery device. In an embodiment, the nasal delivery device is selected from a spray, inhaler, nebulizer or nasal wash.

In one embodiment, the device is a nasal spray. In an embodiment, the nasal spray is a pump spray. Such pumps can comprise an actuating means. In an embodiment, the nasal spray as described herein is a displacement pump. In an embodiment, the he pump is actuated by pressing the actuating means towards the bottle, a piston moves downward in the metering chamber. A valve mechanism at the bottom of the metering chamber will prevent backflow into the dip tube. So the downward movement of the piston will create pressure within the metering chamber which forces the air (before priming) or the liquid outwards through the actuator and generates the spray. When the actuation pressure is removed, a spring will force the piston and actuator to return to its initial position. The metering chamber ensures the right dosing and an open swirling chamber in the tip of the actuator will aerosolize the metered dose. In these pumps no measures are taken to prevent microbial contamination when in use, thus the composition often will contain preservatives, in most cases benzalkonium chloride (BAC) or parabens. In some embodiments, the device uses silver as a preservative. In an embodiment, the device uses a silver wire in the tip of the actuator, a silver coated spring and ball. Such systems are able to keep microorganisms from contaminating the composition between long dosing intervals. Another approach is to use tip seal technology to prevent backflow into the device. In some embodiments, the total volume expelled by each actuation of the device is about 25 to about 200 µL per actuation. In some embodiments, the volume expelled by each actuation is about 50 to about 150 µL per actuation. In one embodiment, the volume expelled by each actuation is about 150 µL per actuation. In one embodiment, the volume expelled by each actuation is about 100 µl per actuation. In one embodiment, the volume expelled by each actuation is about 50 µl per actuation.

In some embodiments, each actuation produces an average particle size of about 10 to about 200 µm. In some embodiments, each actuation produces an average particle size of about 20 to about 180 µm. In some embodiments, each actuation produces an average particle size of about 40 to about 160 µm. In some embodiments, each actuation produces an average particle size of about 60 to about 110 µm.

In some embodiments, the particle size is measured at a velocity of actuation of about 60 mm/s to about 110 mm/s. In some embodiments, the particle size is measured at a velocity of actuation of about 60 mm/s to about 90 mm/s. In some embodiments, the particle size is measured at a velocity of actuation of about 60 mm/s to about 80 mm/s. In some embodiments, the particle size is measured at a velocity of actuation of about 60 mm/s. In some embodiments, the particle size is measured at a velocity of actuation of about 80 mm/s. In some embodiments, the particle size is measured at a distance of about 30 mm to about 80 mm from dispersion point to the vertical laser path. In some embodiments, the particle size is measured at a distance of about 40 mm to about 80 mm. In some embodiment, the particle size is measured at a distance of about 50 mm to about 70 mm. In some embodiment, the particle size is measured at a distance of about 55 mm to about 65 mm. In some embodiments, particle size is measured using actuation of 60 mm/s and a distance of 40 to 70 mm.

In some embodiments, each actuation produces a droplet size distribution Dv10 of at least 10 µm (i.e. 10% of particles have a diameter smaller than 10 µm), or at least 15µum (i.e. 10% of particles have a diameter smaller than 15 µm) at a distance of 40 to 70 nm and 60 mm/s actuation velocity. In some embodiments, each actuation produces a droplet size distribution Dv50 (median value) of at least 50 µm or at least 70 µm, at a distance of 40 to 70 nm and 60 mm/s actuation velocity. In some embodiments, each actuation produces less than 5% or less than 10% of particles of less than 10 µm.

In some embodiments, the distance is measured from the actuation means. In some embodiments, the distance is measured from the dispensing opening in the actuating means.

In an embodiment, the device is an oral delivery device. A person skilled in the art will appreciated that the oral delivery device may be a pulmonary oral delivery device, for example as described in Ibrahim et al (2015) or Chandel et al (2019). In an embodiment, the oral delivery device is selected from a spray, inhaler, nebulizer or oral wash. In an embodiment, the device can deliver one or more doses. In an embodiment, the device is reusable. In an embodiment, the spray is a multi-dose spray.

In an embodiment, the oral device is an oral spray. In an embodiment, the oral spray is a pump spray.

In an embodiment, the device is an inhaler. In an embodiment, the inhaler is a metered-dose inhaler. In an embodiment, the inhaler is a multi-dose inhaler. In an embodiment, the inhaler is a dry powder inhaler. Examples of inhalers can be found in Chandel et al (2019).

In some embodiments, the total volume expelled by each actuation of the inhaler is about 5 to about 150 µL per actuation. In some embodiments, the total volume expelled by each actuation of the inhaler is about 10 to about 110 µL per actuation. In one embodiment, the total volume expelled by each actuation of the inhaler is about 20 µL to about 100 µL per actuation. In one embodiment, the total volume expelled by each actuation of the inhaler is about 100 µL per actuation. In one embodiment, the total volume expelled by each actuation of the inhaler is about 40 µL to about 80 µL per actuation.

In an embodiment, each inhaler actuation produces an average particle size of about 0.01 to about 7 µm. In an embodiment, each nebuliser actuation produces an average particle size of about 0.01 to about 5 µm. In an embodiment, each nebuliser actuation produces an average particle size of about 0.5 to about 5 µm. In an embodiment, each nebuliser actuation produces an average particle size of about 1 to about 5 µm. In an embodiment, each nebuliser actuation produces an average particle size of about 2 to about 4 µm.

In an embodiment, the nebuliser is a jet nebuliser. In an embodiment the nebuliser is an ultrasonic nebuliser. In an embodiment, the nebuliser is a vibrating mesh nebuliser. In an embodiment, the nebuliser is a breath actuated nebuliser. In an embodiment the nebuliser is a breath enhanced nebuliser. In an embodiment, the nebuliser is selected from a: Spiriva Respimat®, the AERx® Pulmonary Drug Delivery System, AeroEclipse® II BAN (Monaghan Medical Corporation), CompAIR™ NE-C801 (OMRON Healthcare Europe BV), I-neb AAD System (Koninklijke Philips NV), Micro Air® NE-U22 (OMRON Healthcare Europe BV), PARI LC® Plus (PARI international), PARI eFlow® rapid (PARI international) and a AKITA® Inhalation System (Activaero)

In some embodiments, the total volume delivered by the nebuliser is about 5 to about 150 µL per actuation. In some embodiments, the total volume delivered is about 10 to about 110 µL per actuation. In one embodiment, the total volume delivered is about 20 µL to about 100 µL per actuation. In one embodiment, the total volume delivered is about 100 µL per actuation. In one embodiment, the total volume delivered is about 40 µL to about 80 µL per actuation.

In an embodiment, the nebuliser produces an average particle size of about 0.01 to about 7 µm. In an embodiment, the nebuliser produces an average particle size of about 0.01 to about 5 µm. In an embodiment, the nebuliser produces an average particle size of about 0.5 to about 5 µm. In an embodiment, the nebuliser produces an average particle size of about 1 to about 5 µm. In an embodiment, the nebuliser produces an average particle size of about 2 to about 4 µm.

### Nasal Sprays

In some embodiments, the macromolecule or composition as described herein is delivered to the nasal cavity and/or nasal mucosa via a nasal spray device. Nasal spray devices of the present invention comprise compositions of the present invention. Actuation of a nasal spray device as described herein comprising a composition as described herein delivers a moisturising protective barrier to the nasal mucous that helps keep the nasal mucous moist and acts as a physical barrier to respiratory viruses.

In an embodiment, compositions as described herein are packaged in a container-closure system comprising an integral spray pump unit that upon activation delivers an accurately metered amount of the composition as a spray. In an embodiment, dispersion as a spray is accomplished by forcing the composition through the nasal actuator and its orifice.

In this embodiment, the container holds about 1 mL to about 50 mL of composition. In this embodiment, the container holds about 4 mL to about 40 mL of composition. In this embodiment, the container holds about 8 mL to about 25 mL of composition. In this embodiment, the container holds about 10 mL to about 20 mL of composition. In this embodiment, the container holds about 10 mL to about 15 mL of composition. In this embodiment, the container holds about 10 mL of composition. In an embodiment, the device is a multi-dose nasal spray device.

In an embodiment, the nasal spray device comprises about 20 to about 120 sprays of the composition . In an embodiment, the nasal spray device comprises about 40 to about 100 sprays of the composition. In an embodiment, the nasal spray device comprises about 60 to about 80 sprays of the composition. In an embodiment, the spray comprises about 80 sprays of the composition. In a preferred embodiment, the metered amount of the composition is about 100 µL.

The nonsterile, pre-filled nasal spray device consists of SPL7013 formulated into a mucoadhesive formulation, containing a small amount of preservative, that adheres at least to the nasal turbinates, nasopharynx, and/or oropharynx. The mucoadhesive composition adheres in the nasal cavity where respiratory viruses that cause colds, flu, and more severe respiratory illness such as COVID-19, first attach and start to multiply. As shown in the experiments described herein SPL7013 has antiviral activities against CoV and RSV and thus can act as a physical barrier to respiratory viruses such as CoV and RSV helping to reduce exposure to respiratory viral pathogens and reducing viral infection load. Reducing infectious viral load may help prevent acquisition or transmission of infection. Due to its physical size and negative charge, SPL7013 is not absorbed into the bloodstream following topical nasal application. The inactivated viruses are eliminated naturally through the nasal mucus.

In an embodiment, the nasal spray device comprises a composition comprising a formulation as described herein. In an embodiment, the formulation is Variant 1 as described herein. In an embodiment, the formulation is Variant 2 as described herein. In an embodiment, the formulation is Variant 3 as described herein. In an embodiment, the formulation is Variant 4 as described herein. In an embodiment, the formulation is Variant 5 as described herein.

In an embodiment, the nasal spray device as described herein, comprising a composition as described herein, delivers a nasal moisture barrier dressing upon actuation. As used herein, a “nasal moisture barrier dressing” refers to a substance applied to the nasal passages (nares) to provide a protective moisture barrier to the external environment and to hydrate and soothe the nasal mucosa. In an embodiment, the nasal moisture barrier dressing has a Global Medical Device Nomenclature code 47679.

In an embodiment, the nasal moisture barrier dressing as described herein comprises one or more of the following features: i) moisturises the nasal mucosa, ii) inactivates CoV, iii) reduces the viral load of CoV.

In an embodiment, the nasal moisture barrier dressing as described herein comprises one or more of the following features: i) moisturises the nasal mucosa, ii) inactivates SARS-CoV-2, iii) reduces the viral load of SARS-CoV-2.

In an embodiment, the nasal moisture barrier dressing as described herein comprises one or more of the following features: i) moisturises the nasal mucosa, ii) inactivates an RSV, iii) reduces the viral load of an RSV.

### Co-Administration

Whilst in some embodiments of the present disclosure, the macromolecules or salts thereof may be the sole active ingredients used, in other embodiments the macromolecule is used are used in combination with one or more further active ingredients, e.g. a further active agent for preventing, treating or reducing the likelihood of infection with a virus. In one embodiment, the virus can infect individuals via the respiratory tract. In one embodiment, the virus can infect individuals via the respiratory tract is selected from: coronavirus, rhinovirus, respiratory syncytial virus, influenza virus, syncytial virus, parainfluenza, adenovirus, metapneumovirus and enterovirus. In one embodiment, the virus is a CoV. In one embodiment, the virus is a RSV.

In an embodiment, the active agent is selected from one or more of: an antiviral active agent, a vaccine, an immunomodulator, a bronchodilator, an antibacterial agent, neuraminidase inhibitors, cap-dependent endonuclease inhibitors, adamantanes, anticoagulants, drugs boosting platelet formation, angiotensin-converting enzyme inhibitors, vitamins, convalescent plasma therapy and/or an anti-inflammatory agent.

As used herein the term “antiviral active agent” refers to a compound that is directly or indirectly effective in specifically interfering with at least one viral action selected from one or more of: virus penetration of a eukaryotic cell, virus replication in a eukaryotic cell, virus assembly, virus release from infected eukaryotic cells, or that is effective in unspecifically inhibiting virus titre increase or in unspecifically reducing a virus titre level in a eukaryotic or mammalian host system. It also refers to an agent that prevents or reduces the likelihood of getting a viral infection.

In an embodiment, the antiviral agent is selected from an antiviral agent described in Gordon et al., 2020 or Ghareeb et al (2021). In an embodiment, the antiviral active agent is selected from one or more of: carrageenan, GM-CSF, IL-6R, CCR5, S protein of MERS, and drugs including, ribavirin, tilorone, favipiravir, Kaletra (lopinavir/ritonavir), Prezcobix (darunavir/cobicistat), nelfinavir, mycophenolic acid, Galidesivir, Actemra, OYA1, BPI-002, Ifenprodil, APN01, EIDD-2801, baricitinib, camostat mesylate, lycorine, Brilacidin, BX-25, amostat, umifenovir, lopinavir, ritonavir, pleconaril, and favipiravir, an interferon ( e.g. IFNβ), antimalarial chloroquine combined and the antibiotic azithromycin.

In an embodiment, the anti-inflammatory agent is selected from one or more of: indomethacin, tocilizumab, JAK inhibitors and ruxolitinib

In an embodiment, the active agent is selected from one or more of: acetaminophen, motavizumab, albuterol, epinephrine, ribavirin and palivizumab. Examples of carrageenan are described for example in CA2696009. In one embodiment, the carrageenan is selected from an iota-carrageenan, kappa-carrageenan and a lambda-carrageenan. In one embodiment, the carrageenan is an iota-carrageenan.

In an embodiment, the active agent reduces the symptoms of one or more RSVs. In an embodiment, when the virus is an RSV the active agent is selected from one or more of: acetaminophen, motavizumab, albuterol, epinephrine, ribavirin and palivizumab.

In one embodiment, the antibacterial agent is an antibiotic. In an embodiment, the antibiotic is a broad-spectrum antibiotic.

In one embodiment, the immunomodulator is an immunosuppressant, a cytokine inhibitor, an antibody, or an immunostimulant. The immunomodulator may suppress inflammation of airways.

The macromolecules or salts thereof may also be used in combination with nonsteroidal anti-inflammatory drug (NSAID). For example, the NSAID may be used to treat the symptoms of a CoVand/or RSV infection, whilst the macromolecule or salt thereof may be used to prevent transmission of the virus to another individual.

The present invention will now be more fully described with reference to the accompanying examples. It should be understood, however, that the description following is illustrative only and should not be taken in any way as a restriction on the generality of the invention described above.

### EXAMPLES

### Example 1: SPL7013 CPE-Based Antiviral Assay

### Methods

virus strains: SARS-CoV-2 hCoV-19/Australia/VIC01/2020 was a gift from Melbourne’s Peter Doherty Institute for Infection and Immunity (Melbourne, Australia). Documentation received with the parent stock indicated that prior to receipt, the virus had been passaged as follows: two passage in Vero cells. A working stock was generated by two further passages in Vero cells in virus growth media, which comprised Minimal Essential Medium without L-glutamine supplemented with 1% (w/v) L-glutamine, 1.0 µg/mL of TPCK-Trypsin (Worthington), 0.2% BSA and 1% Insulin Transferrin Selenium (ITS)). SARS-CoV-2 2019-nCoV/USA-WA1/2020 strain, sourced from BEI Resources (NR-52281). Virus was derived from African green monkey kidney Vero E6 cells or lung homogenates from human angiotensin converting enzyme 2 (hACE2) transgenic mice.

Cells: African Green Monkey Kidney (Vero) cells (ATCC-CCL81) were subcultured to generate cell bank stocks in cell growth medium, which comprised Minimal Essential Medium without L-glutamine supplemented with 10% (v/v) heat-inactivated Fetal Bovine Serum and 1% (w/v) L-glutamine. Cell stocks were frozen at -80° C. overnight and were then transferred to liquid nitrogen for longer term storage. Vero cells were passaged for a maximum of 13 passages, after which a new working cell bank stock was retrieved from liquid nitrogen for further use.

Test and control compound preparation: SPL7013 was dissolved at 40 mg/mL in water, vortexed and visually inspected to confirm complete dissolution. The positive control compound Remdesivir was prepared as a 10 mM stock in DMSO and stored at -20° C.

Preparation of cells for Assay: Vero Cells (ATCC-CCL81) were seeded into 96-well plates for 24 hrs at 2×10⁴ cells/well in 100 µL seeding media (Minimal Essential Medium supplemented with 1% (w/v) L-glutamine, 1% ITS, 0.2% BSA). Plates were incubated overnight at 37° C., 5% CO₂.

Addition of Test and Control Articles to Assay Plate: A volume of 1400 µL virus growth media (Minimal Essential Medium supplemented with 1% (w/v) L-glutamine, 1% ITS, 0.2% BSA, 1 µg/mL TPCK-Trypsin, 1x Pen/Strep) was added to row A, columns 3-11 of a v-bottom skirted PCR plate. Compound (40 mg/mL) was added to column 2 (1300 µL). A 1:3 serial dilution was generated by transfer of 700 µL compound from column 2 to column 3, column 3 to column 4 and continued to column 10 and then discarded. A 50 µL volume from each compound dilution series was added to the rows B-G of an assay plate. SPL7013 was added to assay plates 1 hour pre-infection or 1 hour post-infection.

Addition of Virus: A 50µL volume of SARS-CoV-2 diluted in virus growth media to generate a moi of 0.05, was added to plates. This moi was previously determined to provide 100% CPE in 4 days. Virus was added to rows B, C and D to assess antiviral activity and virus growth media without virus was added to rows E, F and G to assess cytotoxicity. Plates were incubated at 37° C., 5% CO₂ for 4 days prior to assessing CPE.

Cytopathic Effect (CPE) Determination: After incubation for four days, viable cells were determined by staining with MTT. A 100 µL volume of a 3 mg/mL solution of MTT was added to plates and incubated for 4 hours at 37° C. in a 5% CO₂ incubator. Wells were aspirated to dryness using a multichannel manifold attached to a vacuum chamber and formazan crystals solubilised by the addition of 200 µL 100% 2-Propanol at room temperature for 30 minutes. Absorbance was measured at 540 - 650 nm on a plate reader.

Determination of Effective 50% Concentration (EC₅₀): The percent cell protection achieved by the positive control and test articles in virus-infected cells was calculated by the formula as shown below:
Percent cell protection = ([ODt]virus - [ODc]virus / [ODc]mock - [ODc]virus) × 100 Where:
[ODt]virus = the optical density measured in a well examining the effect of a given concentration of test article or positive control on virus-infected cells.

[ODc]virus = the optical density measured in a well examining the effect of the negative control on virus-infected cells.

[ODc]mock = the optical density measured in a well examining the effect of the negative control on mock-infected cells.

$$y = M i n , + \begin{matrix}M a x , - M i n , \\ 1 + {\left(\begin{matrix}E {C}_{50} \\ x\end{matrix}\right)}^{D}\end{matrix}$$

Abbreviations: x, test or control article concentration; y, percent cell protection; Min, minimum; Max, maximum; D, slope coefficient.

The 50% cytotoxic concentration (CC₅₀) is defined as the concentration of the test compound that reduces the absorbance of the mock infected cells by 50% of the control value. The CC₅₀ value was calculated as the ratio of (ODt)mock/(ODc)mock. IDBS XLFit4 Excel Add-in (ID Business Solutions Inc., Alameda, CA) was used to perform the above calculations.

Pre-infection prevention assay: Nine concentrations of SPL7013 (astodrimer sodium) and remdesevir control were prepared by three-fold serial dilution in Assay Media (AM) and added to Vero cells, in triplicate. After 1 hour, 50 µl AM containing the minimum MOI of SARS-CoV-2 hCoV-19/Australia/VIC01/2020 (experimentally determined to provide 100% CPE four days post infection,) was added to compound and virus only control wells [multiplicity of infection (MOI) = 0.05]. An equivalent volume of AM only was added to cytotoxicity and cell only wells. Remdesivir is used as the positive control.

Post infection treatment assay: AM containing the minimum MOI of SARS-CoV-2 hCoV-19/Australia/VIC01/2020 experimentally determined to provide 100% CPE four days post infection, was added to virus only control wells. [multiplicity of infection (MOI) = 0.05]. An equivalent volume of AM only was added to cytotoxicity and cell only wells. After 1 hour nine concentrations of SPL7013 (astodrimer sodium) [and remdesivir control were prepared by three-fold serial dilution in Assay Media (AM) and added to Vero cells, in triplicate.

### Results

The results of the experiment are provided in Table 3. The data demonstrates that the EC₅₀ of SPL7013 is [25 µM and 24 µM] in the micromolar range, indicating it is an effective antiviral agent for prevention and treatment of viral infection. In addition, the SI of around 3.5 for pre and post infective assays indicates selectivity of SPL7013 for SARS-CoV2. Cytotoxicity of controls in this assay was greater than expected, and the SI can be expected to be at or greater than 5 on repeat.

By comparison chloroquine has been reported to have a IC₅₀ of 8 µM and a CC₅₀ of 261, resulting in an SI of 30 against SARS (Keyaerts et al 2004) In more recent work, activity of chloroquine against BetaCoV/Wuhan/WIV04/2019 was EC₅₀ = 1.13 µM; CC₅₀ > 100 µM, SI > 88.50 and Remdesivir was reported with (EC₅₀ = 0.77 µM; CC₅₀ > 100 µM; SI > 129.87) (Cell Research volume 30, pages 269-271(2020 and EC₅₀=0.137 µM by Gilead in EMA Compassionate Use application Procedure No. EMEA/H/K/5622/CU. These assays had less rounds of replication and shorter incubation, so are not directly comparable.

TABLE 3

SPL7013 CPE-based antiviral assay EC₅₀ and CC₅₀ data

| | | |
|---|---|---|
| Compound | EC₅₀ | CC₅₀ |
| SPL7013 1 hour pre-infection | 0.42 mg/mL | 1.47 mg/mL |
| SPL7013 1 hour post-infection | 0.40 mg/mL | 1.29 mg/mL |
| Remdesivir | 5.05 µM | >20 µM |

### Example 2: SPL7013 Virucidal Assay

SPL7013 (astodrimer sodium) at 25 mg/mL was incubated with an equal volume of 10⁵ TCID₅₀/mL units of SARS-CoV-2. The virus-compound mixtures was incubated at 37° C. for 60 minutes, and immediately titrated into Vero cells pre-seeded in 96 well plates, for quantification of infectious viral titre by TCID₅₀ assay. Plates were incubated for three days at 37° C. in a humidified 5% CO₂ atmosphere. Virus-induced CPE were scored visually. The TCID₅₀ of the virus suspension was determined using the method of Reed and Muench (1938). The viricidal effect is quantified as the percent and log reduction in virus titre compared to the SARS-CoV-2 assay media only titre. Controls were: AM, AM+virus and sodium citrate 60 mM as the positive control.

SPL7013 showed viricidal activity in this assay at 25 mg/mL. The assay indicated that the compound stopped all virus growth.

### Example 3: Astodrimer Sodium (SPL7013) Inhibits Replication of SARS-CoV-2 In Vitro

The present study evaluated the antiviral activity of astrodrimer sodium on SARS-CoV-2 in vitro. It was found that astodrimer sodium inhibits replication of SARS-CoV-2 in Vero E6 cells when added to cells 1-hour prior to or 1-hour post infection, with 50% effective concentrations reducing virus-induced cytopathic effect (EC₅₀) ranging from 0.090 to 0.742 µM (0.002 to 0.012 mg/mL). The selectivity index (SI) in these assays was as high as 2197. Astodrimer sodium was also effective in a virucidal evaluation when mixed with virus for 1 hour prior to infection of cells (EC₅₀ 1.83 µM [0.030 mg/mL]). Results from a time of addition study, which showed infectious virus was below the lower limit of detection at all time points tested, were consistent with the compound inhibiting early virus entry steps. The data were similar for all investigations and were consistent with the potent antiviral activity of astodrimer sodium being due to inhibition of virus-host cell interactions.

### Methods

Virus, cell culture, astodrimer sodium and controls: SARS-CoV-2 hCoV-19/Australia/VIC01/2020 was a gift from Melbourne’s Peter Doherty Institute for Infection and Immunity (Melbourne, Australia). Virus stock was generated at 360Biolabs (Melbourne, Australia) by two passages in Vero cells in virus growth media, which comprised Minimal Essential Medium (MEM) without L-glutamine supplemented with 1% (w/v) L-glutamine, 1.0 µg/mL of L-(tosylamido-2-phenyl) ethyl chloromethyl ketone (TPCK)-treated trypsin (Worthington Biochemical, NJ, USA), 0.2% bovine serum albumin (BSA) and 1% insulin-transferrin-selenium (ITS).

SARS-CoV-2 2019-nCoV/USA-WA1/2020 strain, was isolated from an oropharyngeal swab from a patient with a respiratory illness who developed clinical disease (COVID-19) in January 2020 in Washington, US, and sourced from BEI Resources (NR-52281). Virus was derived from African green monkey kidney Vero E6 cells or lung homogenates from hACE2human angiotensin converting enzyme 2 (hACE2) transgenic mice. African green monkey kidney Vero E6 cells or lung homogenates from human angiotensin converting enzyme 2 (hACE2) transgenic mice.

Vero E6 and human Calu-3 cell lines were cultured in Minimal Essential Medium (MEM) without L-glutamine supplemented with 10% (v/v) heat-inactivated fetal bovine serum (FBS) and 1% (w/v) L-glutamine. Vero E6 and Calu-3 cells were passaged for a maximum of 10 passages for antiviral and virucidal studies. Hank’s balanced salt solution (HBBS) with 2% fetal bovine serum (FBS) was used for infection. The 2019-nCoV/USA-WA1/2020 strain antiviral assays were performed with a multiplicity of infection (moi) of 0.1. The virus inoculums for virucidal assays were 10⁴, 10⁵, and 106 pfu/mL with 1.5 mL added to 2.5 × 10⁴ cells/well.

The virus inoculums for virucidal assays were 10⁴, 10³, and 10⁶ pfu/mL. After defined incubation periods, the solution was pelleted through a 20% sucrose cushion (Beckman SW40 Ti rotor) and resuspended in 1.5 mL MEM, which was then added to 2.5×10⁴ cells/well.

Astodrimer sodium was prepared as 86.29 mg/mL or 100 mg/mL in water and stored at 4° C. Astodrimer sodium has a molecular weight of 16581.57 g/mol. The purity of the compound used in these studies was assessed by ultra-high-performance liquid chromatography (UPLC) to be 98.79%. Remdesivir (MedChemExpress, NJ, USA) was used as a positive control in the virus-induced cytopathic effect (CPE) inhibition and time of addition plaque assays.

Virus-induced cytopathic effect inhibition assay: African Green Monkey Kidney (Vero E6, ATCC-CRL1586) cell stocks were generated in cell growth medium, which comprised MEM without L-glutamine supplemented with 10% (v/v) heat-inactivated FBS and 1% (w/v) L-glutamine. Vero E6 cell monolayers were seeded in 96-well plates at 2x10⁴ cells/well in 100 µL growth medium (MEM supplemented with 1% (w/v) L-glutamine, 2% FBS) and incubated overnight at 37° C. in 5% CO₂. SARS-CoV-2 infection was established by using an MOI of 0.05 to infect cell monolayers. Astodrimer sodium or remdesivir were serially diluted 1:3, 9 times and each compound concentration was assessed for both antiviral efficacy and cytotoxicity in triplicate. Astodrimer sodium was added to Vero E6 cells 1 hour prior to infection or 1 hour post-infection with SARS-CoV-2. Cell cultures were incubated at 37° C. in 5% CO₂ for 4 days prior to assessment of CPE. The virus growth media was MEM supplemented with 1% (w/v) L-glutamine, 2% FBS, and 4 µg/mL TPCK-treated trypsin. On Day 4, viral-induced CPE and cytotoxicity of the compound were determined by measuring the viable cells using the methylthiazolyldiphenyl-tetrazolium bromide (MTT) assay (MP Biomedicals, NSW, Australia). Absorbance was measured at 540-650 nm on a plate reader.

Antiviral plaque assay evaluation and nucleocapsid ELISA: For the antiviral evaluation, astodrimer sodium was added to cells 1 hour prior to, at the time of, and 1 hour after exposing the cells to virus. For both the antiviral and virucidal assays, at 6 hours after infection, cells were washed to remove astodrimer sodium and/or any virus remaining in the supernatant, in such way that following initial infection, cell cultures were incubated and supernatants recovered after 16 hours or 4 days. The amount of virus in the supernatants was determined by plaque assay (plaque forming unit [pfu]) and by nucleocapsid enzyme-linked immunosorbent assay (ELISA). The plaque assay used was as described in van den Worm et al (2012), utilizing 2% sodium carboxymethyl cellulose overlay, fixation of cells by 4% paraformaldehyde and staining with 0.1% crystal violet. The nucleocapsid ELISA assay was as described by Bioss Antibodies, USA (BSKV0001). The assessment of astodrimer sodium cytotoxicity occurred on Day 4 by measuring lactate dehydrogenase (LDH) activity in the cytoplasm using an LDH detection kit (Cayman Chemical), with 0.5% saponin used as the positive cytotoxic control.

Virucidal assay: Astodrimer sodium was serially diluted 1:3, 9 times and tested in triplicate wells. SARS-CoV-2 was mixed with diluted astodrimer sodium at a MOI of 0.05 and incubated for 1 hour at 37° C. in 5% CO₂. Virus and compound mixture were added to Vero E6 cell monolayers in 96-well plates and incubated at 37° C. in 5% CO₂ for 4 days. On day 4, the virus-induced CPE was measured by the MTT assay as described above. For the virucidal evaluation, concentrations of astodrimer sodium (0.0046 to 30 mg/mL) were incubated with SARS-CoV-2 2019-nCoV/USA-WA1/2020 for times ranging from 5 seconds to 2 hours. To neutralize the effect of astodrimer sodium, unbound compound was separated from the astodrimer:virus mixture by pelleting the preincubated mixture through a 20% sucrose cushion (Beckman SW40 Ti rotor). The astodrimer sodium-containing supernatant was removed (i.e., neutralizing the effect of SPL7013) and then the pelleted virus was gently resuspended and added to Vero E6 or Calu-3 cell cultures. Virus infection, cell culture and cytotoxicity assessment was as described for the plaque assay described above in the antiviral plaque assay section.

Determination of effective concentration (EC₅₀ and EC₉₀) and cytotoxicity (CC₅₀₎: The concentration of compound that gives a 50% or 90% reduction in viral-induced CPE (EC₅₀ or EC₉₀ respectively) was calculated using the formula described in Example 1. The concentration of compound that resulted in a 50% reduction in cell viability (CC₅₀) after 4 days of culture was also calculated by the formula described in Example 1.

Time of addition assay (TOA): Vero E6 cell monolayers were grown in MEM supplemented with 1% (w/v) L-glutamine, 2% FBS. To ensure robust infection, cell cultures were infected with SARS-CoV-2 at a MOI of 1. The virus was adsorbed for 1 hour at 4° C., then parallel cultures were warmed to 37° C. for 0 min, 15 min, 30 min, 60 min, 2 h, 4 h, 6 h prior to adding 0.345 mg/mL astodrimer sodium, 15 µM hydroxychloroquine, 5 µM remdesivir, or negative control (assay media only). For the 0 min time point, test or control articles were added immediately following virus pre-adsorption. Eight hours after virus infection (the duration of one cycle of replication), virus was harvested from the cells for each time point. The supernatant, containing virus, was retained and the virus titer determined for each time point via virus yield assay.

Virus yield reduction assay: Virus titer from the TOA study was quantified as a median tissue culture infective dose (TCID₅₀) value. TCID₅₀ is a measure of virus titer and represents the titer of a virus that produces infection in 50% of the tissue culture samples. Vero E6 cells monolayers were grown in MEM supplemented with 1% (w/v) L-glutamine, 2% FBS. Virus harvested from each time point was added to three wells and serially diluted three-fold across the plate for a total of nine different virus concentrations. Six of the wells contained assay media alone (i.e., no virus) and served as controls. Plates were incubated for three days and cell monolayers were then observed microscopically with visual scoring of virus-induced CPE used as an endpoint. The TCID₅₀ of the virus suspension was determined using the method of Reed and Muench (1938). Virus yield was expressed as a percentage with respect to virus growth when no compound was added, for each time point.

### Results

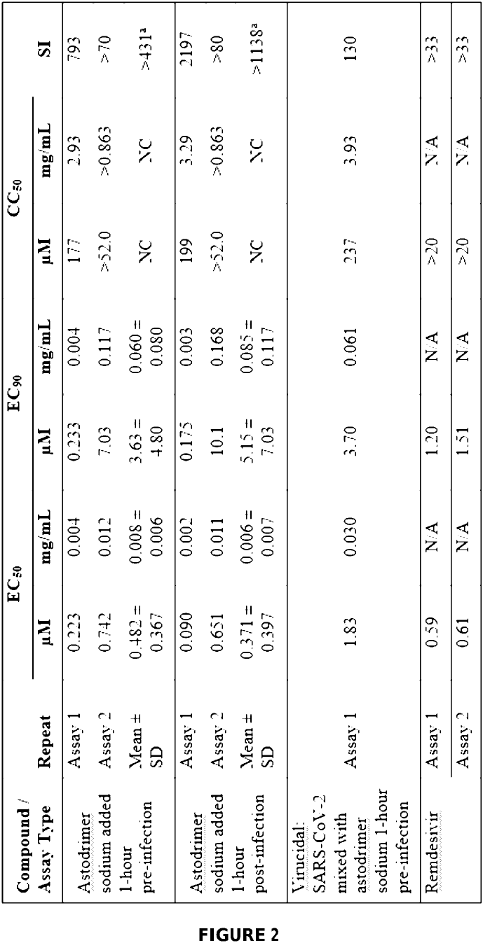

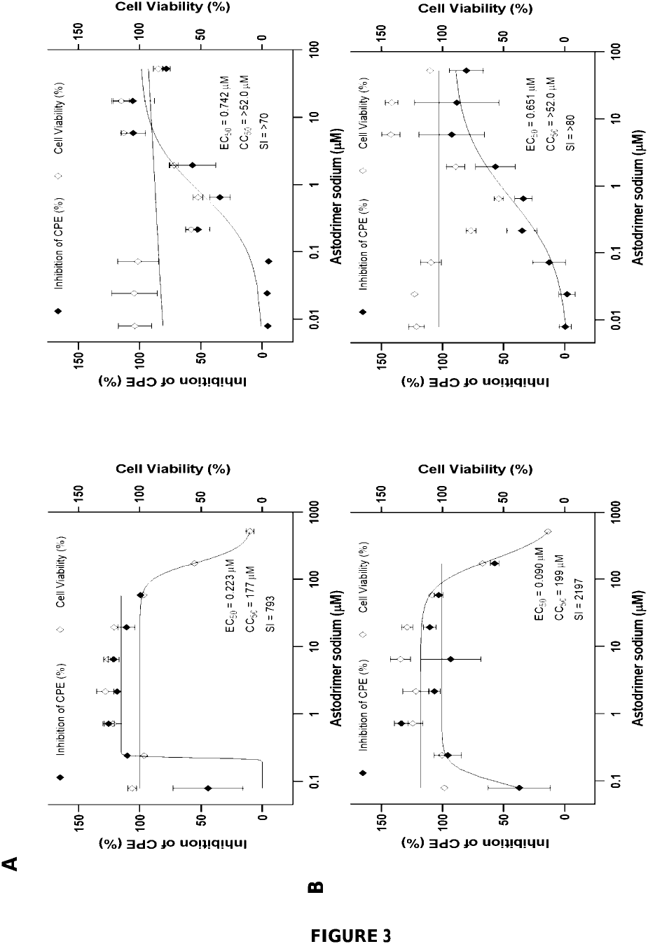

Virus-induced cytopathic effect inhibition assay: In two independent virus-induced CPE inhibition assays, astodrimer sodium inhibited SARS-CoV-2 (hCoV-19/Australia/VIC01/2020) replication in Vero E6 cells in a dose dependent manner (FIG. 2; FIG. 3). Astodrimer sodium inhibited viral replication when added either 1 hour prior to infection, or 1 hour post-infection with SARS-CoV-2. Astodrimer sodium was initially tested in the range of 0.0013 to 8.63 mg/mL (0.078 to 520.4 µM). In the repeat set of assays, astodrimer sodium was tested in the range of 0.0001 to 0.86 mg/mL (0.008 to 52.0 µM) to help further characterize the lower end of the dose response curve. The effective and cytotoxic concentrations, and selectivity indices from the assays are shown individually and as means in FIG. 2 for CPE Determination. The selectivity index (SI) for astodrimer sodium against SARS-CoV-2 in the CPE studies ranged from 793 to 2197 for the initial assays where compound was added 1 hour prior to infection and 1 hour after infection, respectively, and was >70 to >80 in the repeat assays, in which cytotoxicity was not observed up to the highest concentration tested (0.86 mg/mL). The positive control, remdesivir, was also active in the CPE inhibition assay, with a SI of >33.

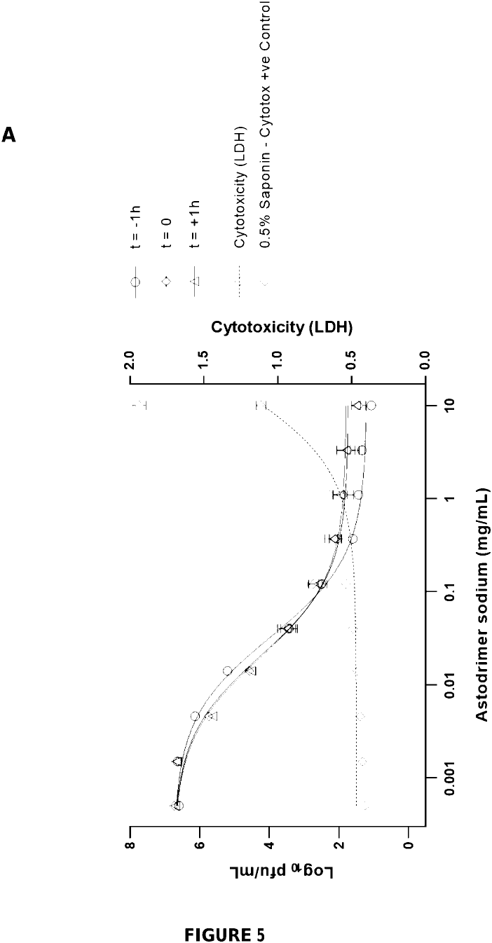

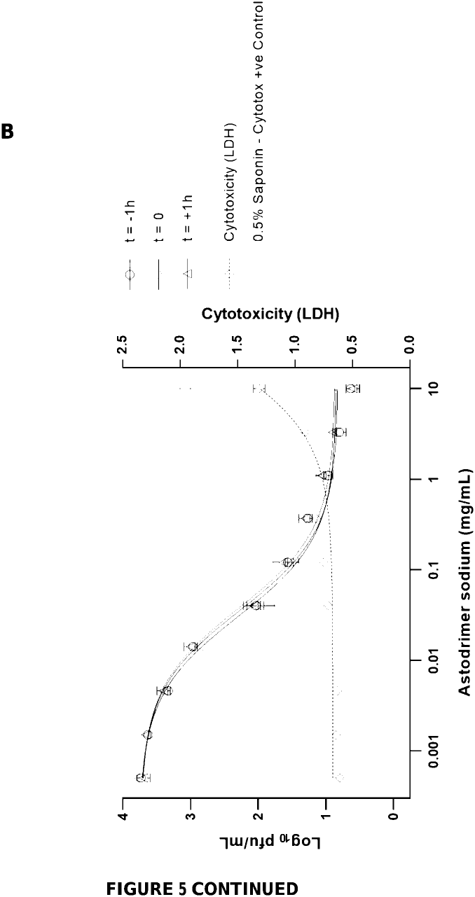

Antiviral efficacy: To determine the ability of astodrimer sodium to inhibit globally diverse SARS-CoV-2 strains, the compound was evaluated against the 2019-nCoV/USA-WA1/2020 virus in Vero E6 cells and human Calu-3 cells. Antiviral readouts were based on virological endpoints of infectious virus or viral nucleocapsid released into the supernatant post-infection. As shown in Table 4 and FIG. 5, astodrimer inhibited the 2019-nCoV/USA-WA1/2020 strain with an EC₅₀ 0.019 to 0.032 mg/mL and 0.0320 to 0.037 mg/mL for infectious virus release as determined by plaque assay in Vero E6 cell for Calu-3 cells, respectively. These data are consistent with the inhibition by astodrimer of the replication of the Australian SARS-CoV-2 isolate in vitro. The dose response data for the nucleocapsid released into the supernatant by ELISA were similar to the infectious virus release data in each cell line (data not shown). The positive control, remdesivir, was also active in the plaque assay.

TABLE 4

Antiviral efficacy, measured by a reduction in mean infectious virus (Log10 pfu/mL), and selectivity of astodrimer sodium against SARS-CoV-2 (2019-nCoV/USA-WA1/2020) on Day 4 post-infection

| Compound / Assay Type | Cell Line | EC₅₀ (mg/mL) | CC₅₀ (mg/mL) | SI |
|---|---|---|---|---|
| Astodrimer sodium added 1-hour pre-infection | Vero E6 | 0.032 | 15.09 | 472 |
| | Calu-3 | 0.037 | 21.76 | 588 |
| Astodrimer sodium added at time of infection | Vero E6 | 0.020 | 15.09 | 755 |
| | Calu-3 | 0.035 | 21.76 | 622 |
| Astodrimer sodium added 1-hour post-infection | Vero E6 | 0.019 | 15.09 | 794 |
| | Calu-3 | 0.030 | 21.76 | 725 |
| Remdesivir added 1-hour post-infection | Vero E6 | 0.791 µM | N/A | N/A |
| | Calu-3 | 0.589 µM | N/A | N/A |

EC₅₀=50% effective concentration; CC₅₀=50% cytotoxic concentration; SI=selectivity index (CC₅₀/EC₅₀); N/A=not applicable

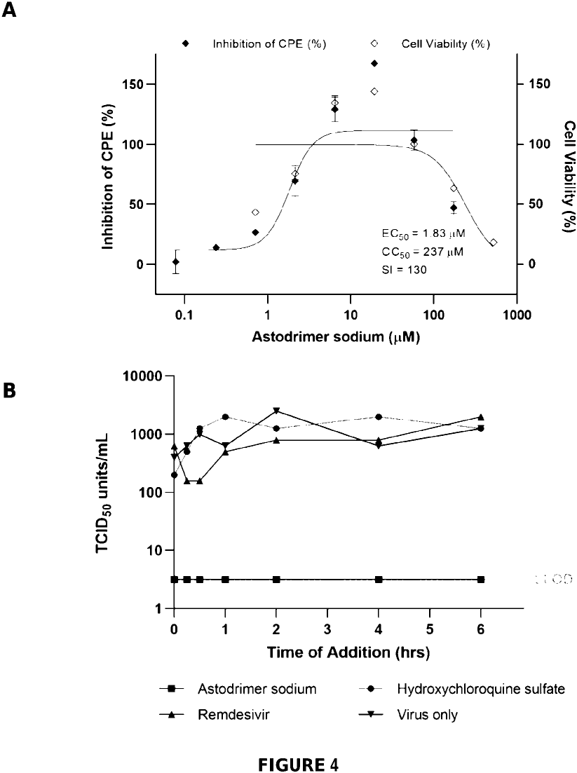

Virucidal efficacy: A study was performed to determine if astodrimer sodium was able to reduce viral infectivity by irreversibly inactivating SARS-CoV-2 prior to infection of Vero E6 cells. Astodrimer sodium treatment demonstrated a similar level of antiviral efficacy to the CPE studies (FIG. 2; FIG. 4A) with an EC₅₀ of 1.83 µM (0.030 mg/mL) and SI of 130; n=1. Assessment for antiviral activity at early middle and late stages of virus replication by adding compounds at different times post-infection (0 min, 15 min, 30 min, 1 h, 2 h, 4 h, and 6 h). The amount of virus secreted into the supernatant at 8 hours post-infection was determined by TCID₅₀. Virucidal assays investigated if astodrimer sodium was able could reduce viral infectivity by irreversibly inactivating SARS-CoV-2 prior to infection of Vero E6 cells and human airway Calu-3 cells. Following incubation of virus with astodrimer for up to 2 hours and neutralization of astodrimer, the astodrimer-exposed virus was added to cell cultures. After either 16 hours or 96 hours (Day 4), the cell culture supernatant was collected for assessment of progeny viral infectivity as determined by the amount of secreted infectious virus and nucleocapsid. The SARS-CoV-2 replication lifecycle is completed in approximately 8 hours (Ogando et al., 2020) and in these studies, we sampled at 16 hours (2 lifecycles) or Day 4 (12 lifecycles) post-infection. Enabling a possible 12 rounds of infection, the Day 4 (96 hour) sampling time point identified that exposure of 106 pfu/mL SARS-CoV-2 to astodrimer sodium for 1 to 2 hours resulted in a dose-dependent reduction in viral infectivity, with 10 to 30 mg/mL astodrimer sodium achieving up to >99.999% (>5 log10) reduced infectivity in Vero E6 cells and >99.9% (>3 log10) reduced infectivity in Calu-3 cells compared to untreated virus (data not shown). SARS-CoV-2 infectivity was also reduced by up to >99.999% in Vero E6 cells when the incubation time of astodrimer (10 to 30 mg/mL) with 106 pfu/mL virus was reduced to 15 to 30 minutes (data not shown).

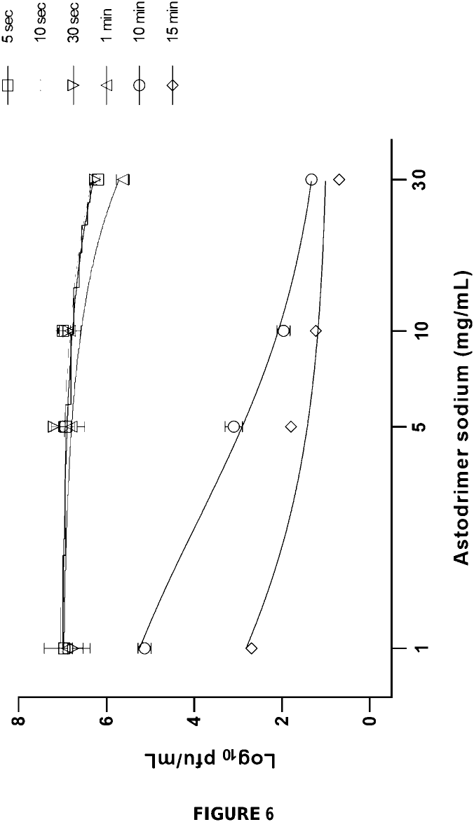

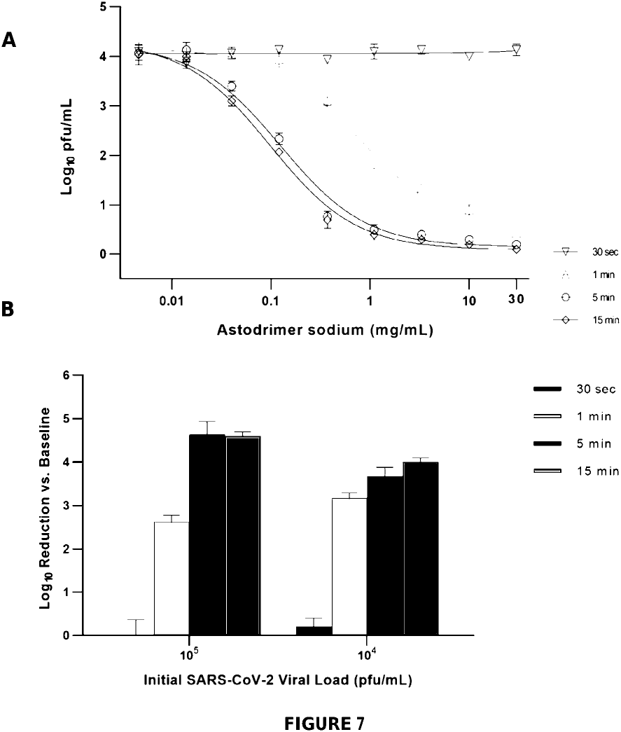

Incubation of astodrimer sodium (1 to 30 mg/mL) with viral inoculums of 10⁴, 10⁵ and 10⁶ pfu/mL for as little as 5 seconds resulted in evidence of reduced infectivity, with 10 to 15 minutes exposure being sufficient to achieve >99.9% reduction in virus infectivity, and greater reduction achieved with lower viral inoculum (>99.999%, 10⁴ pfu/mL viral inoculum, 10 to 30 mg/mL astodrimer sodium, and 10 to 15 min incubation time) (Table 5, FIG. 6). When assessed 16 hours post-infection of cells with astodrimer-exposed virus, it was found that >10 mg/mL astodrimer sodium inactivated >99.9% SARS-CoV-2 (104 pfu/mL) within as little as 1 minute of exposure (Table 6, FIG. 7). Exposure of astodrimer sodium to virus for 30 seconds had no detectable virucidal effect.

TABLE 5

Virucidal efficacy of 10 mg/mL astodrimer sodium against SARS-CoV-2 (2019-nCoV/USA-WA1/2020), measured by a reduction in mean infectious virus (Log₁₀ pfu/mL), at 96 hours post-infection

| Viral Load (pfu/mL) | Virus:Astodrimer Incubation Time | Reduction vs. Virus Control (Log₁₀± SD) | Reduction vs. Virus Control (%) |
|---|---|---|---|
| 10⁶ | 5 sec | 0.10 ± 0.20 | 20.567 |
| | 10 sec | 0.03 ± 0.06 | 7.388 |
| | 30 sec | 0.10 ± 0.10 | 20.567 |
| | 1 min | 0.33 ± 0.12 | 53.584 |
| | 10 min | 2.20 ± 0.10 | 99.369 |
| | 15 min | 3.67 ± 0.23 | 99.979 |
| 10⁵ | 5 sec | 0.33 ± 0.21 | 53.584 |
| | 10 sec | 0.23 ± 0.06 | 41.566 |
| | 30 sec | 0.30 ± 0.17 | 49.881 |
| | 1 min | 0.47 ±0.21 | 65.855 |
| | 10 min | 3.70 ± 0.26 | 99.980 |
| | 15 min | 4.60 ± 0.10 | 99.998 |
| 10⁴ | 5 sec | -0.13 ± 0.21 | -35.936 |
| | 10 sec | 0.07 ± 0.29 | 14.230 |
| | 30 sec | 0.10 ± 0.10 | 20.567 |
| | 1 min | 0.10 ± 0.00 | 20.567 |
| | 10 min | 5.07 ± 0.25 | >99.999 |
| | 15 min | 5.83 ± 0.12 | >99.999 |

Shading indicates data points where virucidal efficacy is >99.9% (3 log₁₀ reduction) vs. virus control; virus control=untreated virus, 0 mg/mL astodrimer sodium; SD=standard deviation

TABLE 6

Virucidal efficacy of 10 mg/mL astodrimer sodium against SARS-CoV-2 (2019-nCoV/USA-WA1/2020), measured by a reduction in mean infectious virus (Log₁₀ pfu/mL), at 16 hours post-infection

| Viral Load (pfu/mL) | Virus:Astodrimer Incubation Time | Reduction vs. Virus Control (Log₁₀ ± SD) | Reduction vs. Virus Control (%) |
|---|---|---|---|
| 10⁵ | 30 sec | 0.00 ± 0.36 | 0.000 |
| | 1 min | 2.63 ± 0.15 | 99.767 |
| | 5 min | 4.63 ± 0.31 | 99.998 |
| | 15 min | 4.60 ± 0.10 | 99.998 |
| 10⁴ | 30 sec | 0.20 ± 0.20 | 36.904 |
| | 1 min | 3.17 ± 0.12 | 99.932 |
| | 5 min | 3.67 ± 0.21 | 99.979 |
| | 15 min | 4.00 ± 0.10 | 99.990 |

Shading indicates data points where virucidal efficacy is >99.9% (3 log₁₀ reduction) vs. virus control; virus control =untreated virus, 0 mg/mL astodrimer sodium; SD=standard deviation

Time of addition assay (TOA): To further investigate the mechanism of action of astodrimer sodium, a TOA study was performed. Compound was added to virus-infected cells at early, middle and late stages of the SARS-CoV-2 replication lifecycle, which is completed in approximately 8 hours. The addition of 0.345 mg/mL astodrimer sodium at times ranging from 0 min to 6 h post-infection resulted in virus levels below the lower limit of detection at every time point (FIG. 4B). This finding was in contrast to detectable infectious virus levels at all equivalent time points in the positive control (remdesivir and hydroxychloroquine sulfate) and virus only cultures. Hydroxychloroquine sulfate had no discernible effect on virus replication at any time point at 15 µM. Remdesivir (5 µM, ~5-10 times the EC₅₀) inhibited virus replication by <1 log₁₀ TCID₅₀ when added within 15 or 30 min post-infection.

### Discussion

Astodrimer sodium demonstrated potent antiviral activity against diverse SARS-CoV-2 cells in vitro. Antiviral activity was demonstrated by reduction in CPE, release of infectious virus and release of viral nucleocaspid protein. Antiviral activity was demonstrated when astodrimer sodium was added to cells prior to infection of cells and when the compound was added to cells already exposed to SARS-CoV-2. Irreversible virucidal activity was demonstrated when astodrimer sodium was mixed with virus for as little as 1 minute.

Of note is a significantly high SI for astodrimer sodium in the antiviral assays relative to other antiviral compounds under investigation for SARS-CoV-2 activity (Pizzorno et al., 2020).

Remdesivir was used as the antiviral positive control for the CPE inhibition and antiviral assays and the experimental EC₅₀ was consistent with published data generated with a different clinical isolate of SARS-CoV-2 (Wang et al., 2020).

The antiviral data are consistent with astodrimer sodium being a potent inhibitor of early events in the virus lifecycle. The virucidal assay data suggest that astodrimer sodium antiviral activity was consistent with binding to virus, thereby irreversibly inactivating virus and blocking infection.

The complete abolition of virus infection at all time points in the TOA assay is also consistent with astodrimer sodium being a potent antiviral agent that inhibits the early phase of virus infection and replication.

The virucidal activity of astodrimer sodium demonstrated that it irreversibly inhibits the early phase of virus infection and replication. These findings suggest potent inhibition of viral attachment, fusion and entry of the virus, which prevents virus replication and release of infectious virus progeny. These findings suggest potent inhibition of viral attachment, fusion and entry of the virus, which prevents virus replication and release of infectious virus progeny.

Data from the current studies, indicate that the compound exerts its antiviral activity against geographically diverse SARS-CoV-2 isolates by interfering with the early virus-cell recognition events. Astodrimer sodium is a potent virucidal agent that reduces the infectivity of SARS-CoV-2 by >99.9% after 1 minute of exposure to the virus. These studies support astodrimer sodium being able to prevent early virus entry steps such as attachment, thereby reducing or preventing viral infection or cell-cell spread.

An antiviral agent such as astodrimer sodium that blocks binding of the virus to target cells would be useful as a preventive and/or a therapeutic agent against SARS-CoV-2. These antiviral studies suggest that reformulation of astodrimer sodium for delivery to the respiratory tract may be an effective preventive strategy to block SARS-CoV-2 transmission and augment other protective and therapeutic strategies.

### Example 4: Evaluation of the Virucidal Properties of SPL7013 Against Three Human Coronaviruses (hCoV-229E, hCoV-NL63 and hCoV-OC43)

A Virucidal Suspension Test (In-Vitro Time-Kill method) was used to evaluate the virucidal properties of SPL7013 versus hCoV-229E (ATCC #VR-740), hCoV-NL63 (ZeptoMetrix Corp. #0810228CF), and hCoV-OC43 (ZeptoMetrix Corp. #0810024CF). Test viruses used for this study were from BSLI high titer virus stock.

On the day of use, aliquots of stock viruses were removed from a -70° C. freezer and thawed prior to use in testing. The percent and log₁₀ reductions from the initial population of the viral strains were determined following exposure to the test product for 60 seconds and 60 minutes. The viral titres were determined using a 50% tissue culture infectious dose (TCID50) calculation (the Quantal test).

### Methods

Cell culture: The cell lines used were human lung fibroblasts (MRC-5; ATCC #CCL-171), green monkey epithelial kidney cells (Vero; ATCC #CCL-81) and human colon adenocarcinoma, epithelial (HCT-8; ATCC #CCL-244). Cells were maintained as monolayers in disposable cell culture labware and were used for the Virucidal Suspension Test. Prior to testing, host cell cultures were seeded onto 24-well cell culture plates. MRC-5 cells were approximately 90% confluent and less than 48 hours old before inoculation with Coronavirus strain 229E. Vero cells were approximately 90% confluent and less than 48 hours old before inoculation with Coronavirus strain NL63. HCT-8 cells were approximately 80% confluent and less than 48 hours old upon inoculation with Coronavirus strain OC43. The growth medium (GM) was replaced by maintenance medium (MM) to support virus propagation.

Test product: SPL7013 aqueous solution 99.1 m/mL. An aliquot of 15.99 mL of Test product was added into 34.01 mL of sterile water to obtain a concentration of 31.95 mg/mL. The final concentration tested was 28.76 mg/mL.

Virucidal suspension test: The virucidal suspension test included the parameters described in Table 7.

Test: A 0.5 mL aliquot of test virus(s) was added to a vial containing 4.5 mL of the test product concentration. The test virus(s) was exposed to the test product(s) for 60 seconds and 60 minutes. Immediately after exposure(s), the test virus(s)/product suspensions was neutralized in Fetal Bovine Serum, mixed thoroughly, and serially diluted in MM. Each dilution was plated in four replicates.

Virus control: A 0.5 mL aliquot of test virus(s) was added to 4.5 mL of MM and exposed for 60 seconds and 60 minutes at ambient temperature. The subsequent test virus dilution was made in MM and serially diluted in MM. Each dilution was plated in four replicates.

Cytotoxicity control: A 0.5 mL aliquot of MM was added to a vial containing 4.5 mL of the test product concentration(s). The MM/product mixture was neutralized in Fetal Bovine Serum, mixed thoroughly and serially diluted in MM. Each dilution was plated in four replicates.

Neutralization control: A 0.5 mL aliquot of MM was added to a vial containing 4.5 mL of the undiluted test product. The MM/product mixture was diluted to 1:10 in Fetal Bovine Serum. An aliquot of the virus(s) was added to the neutralized product and thoroughly mixed and exposed to the neutralized product for 10 to 20 minutes. Subsequent 10-fold dilutions of neutralized test product/virus suspension was made in MM. Each dilution was plated in four replicates.

Neutralizer toxicity control: The effect of the neutralizer on virus infectivity was assessed by adding virus to the neutralizer (Fetal Bovine Serum) alone followed by exposure for 10 to 20 minutes. Subsequent 10-fold dilutions of neutralized test product/virus suspension were made in MM. Each dilution was plated in four replicates.

Cell culture control: Intact cell culture served as the control of cell culture viability. The GM was replaced by MM in all cell control wells.

The plates were incubated in a CO₂ incubator for 10 to 14 days at the appropriate for each virus temperature in a CO₂ incubator. Cytopathic/cytotoxic effect was monitored using an inverted compound microscope.

TABLE 7

Parameters of the Virucidal suspension test

| | | |
|---|---|---|
| Parameter | Summary | Plating Replicates |
| Virucidal suspension test | Virus + Test Product →Exposure → Neutralization →Dilution →Plating | 4 per group |
| Virus Control | Virus + Diluent → Exposure → Dilution → Plating | 4 per group |
| Neutralization Control | Test Product + Diluent → Neutralization → Dilution → Plating | 4 per group |
| Cytotoxicity Control | Test Product + Diluent → Neutralization → Virus inoculation → Dilution → Plating | 4 per group |
| Neutralizer Toxicity Control | Virus + Diluent → Neutralization → Dilution → Plating | 4 per group |
| Cell Culture Control | Maintenance medium | 4 per group |

### Results

The virucidal data for SPL7013 against the three human CoV strains are provided in Table 8, Table 9 and Table 10. The SPL7013 aqueous solution 99.91 mg/mL reduced the infectivity of hCoV-229E by 0.75 log₁₀ ( 82.22%) following 60-second and 60-minute exposures; reduced the infectivity of hCoV-NL63 by 0.50 log₁₀(68.38%) following a 60-second exposure and by 0.75 log₁₀ (82.22%) following a 60-minute exposure; and reduced the infectivity of hCoV-OC43 by 0.50 log₁₀ (68.38%) following 60-second and 60-minute exposures. These result shows that SPL7013 is active against multiple human CoV strains.

TABLE 8

SPL7013 aqueous solution Virucidal Activity against Coronavirus strain 229E (ATCC #VR-740)

| Dilution (- Log₁₀) | Virus Control | | Test | | NTC | NC | CTC | CC |
|---|---|---|---|---|---|---|---|---|
| | 60 seconds | 60 minutes | 60 seconds | 60 minutes | | | | |
| | | | | | | | | 0000 |
| -2 | NT | NT | ++++ | ++++ | NT | NT | 0000 | N/A |
| -3 | ++++ | ++++ | ++++ | ++++ | ++++ | ++++ | 0000 | |
| -4 | ++++ | ++++ | ++++ | ++++ | ++++ | ++++ | 0000 | |
| -5 | ++++ | ++++ | ++++ | +0+0 | ++++ | +++0 | NT | |
| -6 | +0+0 | ++00 | 0000 | 000+ | ++00 | 0++0 | NT | |
| -7 | 000+ | 0000 | 0000 | 0000 | 0+00 | 0000 | NT | |
| TCID₅₀ (log₁₀) | 6.25 | 6.00 | 5.50 | 5.25 | 6.25 | 5.75 | 1.50 | |
| Log₁₀ Reduction | N/A | | 0.75 | 0.75 | N/A | | | |
| Percent Reduction | | | 82.22 % | 82.22 % | | | | |

+ CPE (cytopathic/cytotoxic effect) present; 0 CPE (cytopathic/cytotoxic effect) not detected CC Cell Control; CTC Cytotoxicity Control; NC Neutralization Control; NTC Neutralizer Toxicity Control; NT Not tested; N/A Not applicable.

TABLE 9

SPL7013 aqueous solution Virucidal Activity against : Coronavirus strain NL63 (ZeptoMetrix Corp. #0810228CF)

| Dilution (- Log₁₀) | Virus Control | | Test | | NTC | NC | CTC | CC |
|---|---|---|---|---|---|---|---|---|
| | 60 seconds | 60 minutes | 60 seconds | 60 minutes | | | | |
| | | | | | | | | 0000 |
| -2 | NT | NT | ++++ | ++++ | NT | NT | 0000 | N/A |
| -3 | ++++ | ++++ | ++++ | ++++ | ++++ | ++++ | 0000 | |
| -4 | ++++ | ++++ | ++++ | ++++ | ++++ | ++++ | 0000 | |
| -5 | ++++ | ++++ | +++0 | ++00 | ++0+ | ++++ | NT | |
| -6 | 0+00 | +000 | 0000 | 0000 | 0000 | 0000 | NT | |
| -7 | 0000 | 0000 | 0000 | 0000 | 0000 | 0000 | NT | |
| TCID₅₀ (log₁₀) | 5.75 | 5.75 | 5.25 | 5.00 | 5.25 | 5.50 | 1.50 | |
| Log₁₀ | | | 0.50 | 0.75 | | | | |
| Reduction | N/A | | 68.38% | 82.22 % | N/A | | | |
| Percent Reduction | | | | | | | | |

+ CPE (cytopathic/cytotoxic effect) present; 0 CPE (cytopathic/cytotoxic effect) not detected CC Cell Control; CTC Cytotoxicity Control; NC Neutralization Control; NTC Neutralizer Toxicity Control; NT Not tested; N/A Not applicable.

TABLE 10

SPL7013 aqueous solution Virucidal Activity against : Coronavirus strain OC43 (ZeptoMetrix Corp. #0810024CF)

| Dilution (- Log₁₀) | Virus Control | | Test | | NTC | NC | CTC | CC |
|---|---|---|---|---|---|---|---|---|
| | 60 seconds | 60 minutes | 60 seconds | 60 minutes | | | | |
| | | | | | | | | 0000 |
| -2 | NT | NT | ++++ | ++++ | NT | NT | 0000 | N/A |
| -3 | ++++ | ++++ | ++++ | ++++ | ++++ | ++++ | 0000 | |
| -4 | ++++ | ++++ | ++++ | +++0 | ++++ | ++++ | 0000 | |
| -5 | ++++ | ++0+ | ++++ | 0+++ | +++0 | ++++ | NT | |
| -6 | 00++ | +00+ | 0000 | +000 | 000+ | 0000 | NT | |
| -7 | 0000 | 0000 | 0000 | 0000 | 0000 | 0000 | NT | |
| TCID₅₀ (log₁₀) | 6.00 | 5.75 | 5.50 | 5.25 | 5.50 | 5.50 | 1.50 | |
| Log₁₀ Reduction | N/A | | 0.50 | 0.50 | N/A | | | |
| Percent Reduction | | | 68.38% | 68.38 % | | | | |

+ CPE (cytopathic/cytotoxic effect) present; 0 CPE (cytopathic/cytotoxic effect) not detected; CC Cell Control; CTC Cytotoxicity Control; NC Neutralization Control; NTC Neutralizer Toxicity Control; NT Not tested; N/A Not applicable.

### Example 5: Evaluation of the Virucidal Properties of SPL7013 Against the SARS-CoV-2 Strain Slovakia/SK-BMC5/2020

The virucidal properties of SPL7013 were assessed against the SARS-CoV-2 strain Slovakia/SK-BMC5/2020. This strain was isolated from a COVID-19 patient from Slovakia in March 2020.

### Methods

Cells: Vero E6/TMPRSS2 non-human primate kidney epithelial cells (National Institute for Biological Standards and Controls, UK).

Virus: Slovakia/SK-BMC5/2020 was supplied through the European Virus Archive goes Global (Evag) platform. SARS-Cov-2 was amplified and titered on Vero E6/TMPRSS2 cell line.

Cytotoxicity and viral quantification (Experiment 1): Cells were counted and their viability assessed using Vi-Cell automatic apparatus. Cells were seeded at ~15,000 cells/well. Cells were pre-treated as follows for 1 h at 37° C. Eight doses of SPL7013 (10, 3.3, 1.1, 0.37, 0.12, 0.04, 0.014, 0.0046 mg/mL) were prepared in cell media. The reference compound (Apilimod) was prepared at three concentrations (1000, 300 and 100 nM). Slovakia/SK-BMC5/2020 was subsequently added at one MOI ( ~0.01) in a volume of 10 µL on pre-treated cells and incubated for 48 hrs in a 37° C. incubator. Supernatants were collected for viral load determination (RT-qPCR).

The CellTiter 96® AQueous Non-Radioactive Cell Proliferation Assay (MTS/PMS assay) was performed on both a plate control (without virus) and on plates treated and infected as described above. The assay (Promegaref# G5430) was performed according to the manufacturer protocol. Supernatant was removed from wells for PCR reactions and a volume of 100 µL of fresh cell medium and a volume of 20 µL of MTS/PMS reagent was added to each well. Absorbance was recorded every hour for four hours.

Quantification of viral load by RTqPCR was performed at the end of the experiment using the ORFlab gene. RNA was extracted using the Viral Kit (Macherey-Nagel,#740709). RNA was frozen at -20° C. until use. RT-PCR was performed with the SuperScript™ III One-Step QRT-PCR System kit (commercial kit#1732-020, Life Technologies) using a Bio-Rad CFX384™ instrument and adjoining software.

Microscopy (Experiment 2): Cells were seeded at ~15,000 cells/well. Cells were pre-treated as follows for 1h at 37° C. Eight doses of SPL7013 (10, 3.3, 1.1, 0.37, 0.12, 0.04, 0.014, 0.0046 mg/mL) were prepared in cell media. The reference compound (Apilimod) was prepared at three concentrations (1000, 300 and 100 nM). Next, Slovakia/SK-BMC5/2020 was subsequently added at 1 MOI (~0.5) in a volume of 10 µL to the cells and incubated for 6 hrs at 37° C. Cells were fixed for immunofluorescence staining using the SARS-CoV-2 (2019-nCoV) Nucleoprotein / NP Antibody, rabbit Mab primary antibody (Sino Biological, #40143-R019; 1:8000 dilution) and imaged using Operetta.FFU/mL.

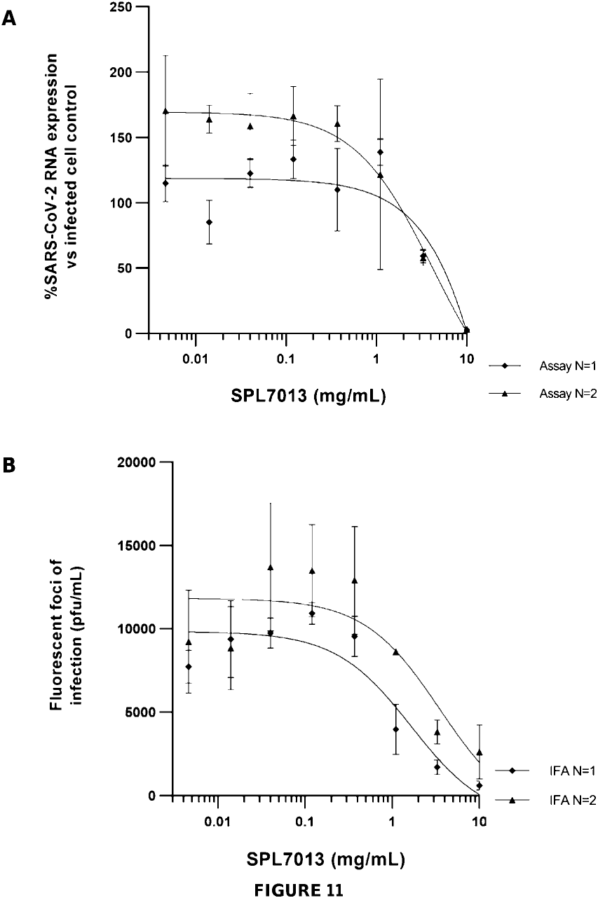

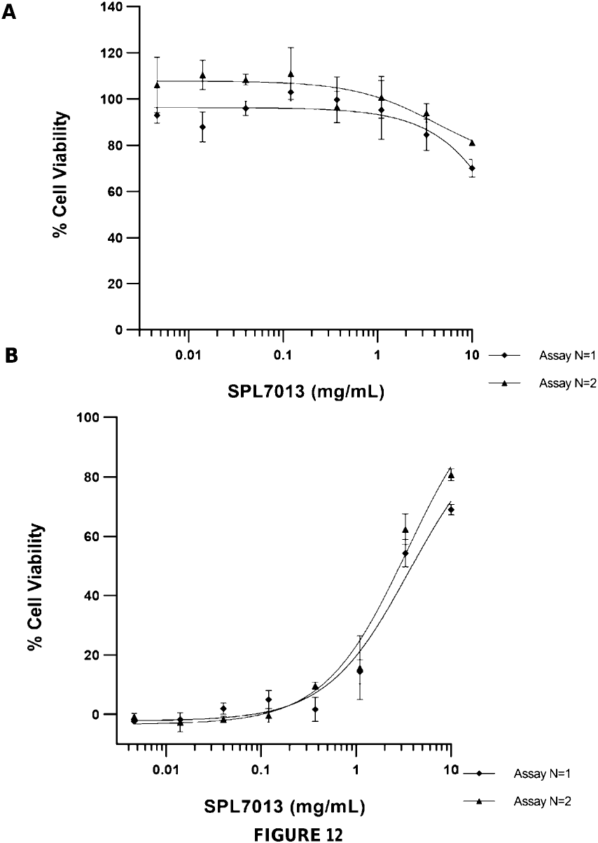

Results are shown in FIGS. 11 and 12.

### Example 6: In Vivo Assessment of SPL7013 Against SARS-CoV-2 Infection in hACE2 Transgenic Mice Following Nasal Administration for 7 Days

### Method

Briefly, 4 groups of 5 animals human ACE2 transgenic mice K18-hACE2 (available from the Jackson Laboratory, B6.Cg-Tg(K18-ACE2)2Prlmn/J, stock number 034860) approximately 6-8 weeks old were used to assess the effect of SPL7013 on viral load in vivo.

Animals in these groups were inoculated intranasally with 25 µL per nostril of a virus suspension containing 10⁴ PFU/µL of SARS-CoV-2 (total challenge: 5×10⁵ PFU) (2019-nCoV/USA-WAl/2020 strain). Animals were dosed 25 µL/nostril (total 50 µL) of either PBS (phosphate buffered saline) or 1%, 3% or 5% SPL7013 in PBS for 7 days, resulting in total daily administrations of 0, 0.5, 1.5 and 2.5 mg of SPL7013, respectively (on days 1 to 6). The first dose of SPL7013 was administered on Day 0, 5 minutes prior to virus inoculation, and a subsequent dose was administered on Day 0, 5 minutes after virus inoculation, and once on Day 1 to Day 6, at the same time each day. Animals were euthanised on Day 7. Status of infection was determined by measuring viral load by quantitative polymerase chain reaction (qPCR) from samples of nasal swabs (Day 7).

### Results

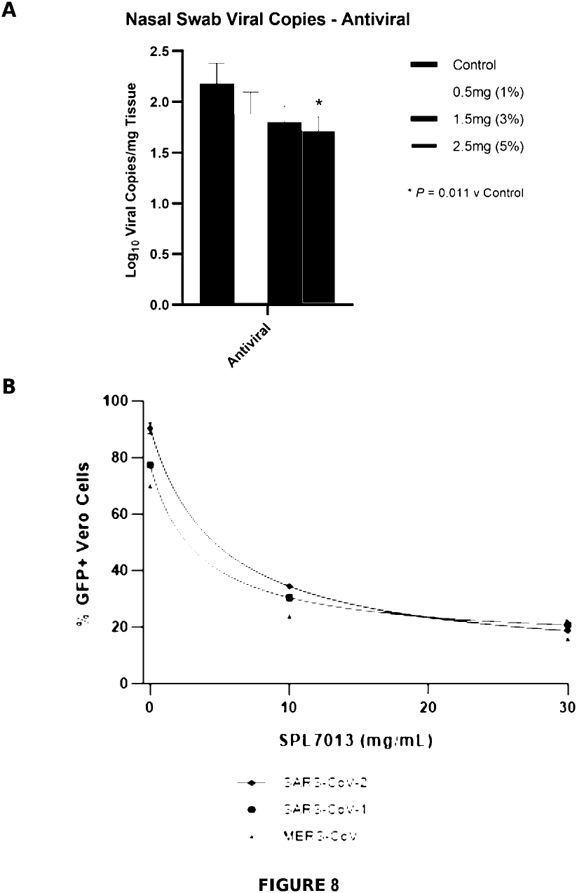

There was a dose dependent decrease in viral copies (qPCR) in nasal swabs at Day 7, which reached statistical significance vs control at the highest dose level (FIG. 8A). This data indicates that when administered nasally, SPL7013 can reduce the nasally acquired SARS-CoV-2 viral load in a dose dependent manner. A dose of 2.5 mg/day was most effective at reducing viral load.

### Example 7: Evaluation of Antiviral Properties of SPL7013 Against Severe Acute Respiratory Syndrome Coronavirus (SARS) and Middle East Respiratory Syndrome (MERS) Coronavirus

### Methods

Cell culture and virus: HEK-293T cells expressing hACE2^{+,} and hTMPRSS2⁺ and Vero E6 cells (ATCC-CRL1586) were cultured in Minimal Essential Medium (MEM) without L-glutamine supplemented with 10% (v/v) heat-inactivated fetal bovine serum (FBS) and 1% (w/v) L-glutamine. Hank’s balanced salt solution (HBBS) with 2% FBS was used for infection. Pseudotyped SARS-CoV-1 (Urbani), SARS-CoV-2 (Wuhan-Hu-1), MERS-CoV (HCoV-EMC) reporter virus particles (RVPs) were generated by Integral Molecular (catalogue numbers RVP-801, RVP-701, RVP-901, respectively). RVPs display antigenically correct spike protein on a heterologous virus core and carry a modified genome that expresses a convenient optical reporter gene, green fluorescent protein (GFP), within 24 hours of cellular infection. SARS-CoV-2 spike receptor binding domain (RBD) recombinant protein with an mFc-tag (SARS-CoV-2 Spike RBD (318-541) Recombinant Protein (mFc-Tag) #41701, Cell Signalling Technology) was used per manufacturer’s instructions.

Assay for SARS-CoV-1, SARS-CoV-2 and MERS-CoV pseudotyped GFP reporter lentiviral particles: 100,000 cells of Vero 6 cells were seeded per well of a 96-well plate for the assay. Cells were seeded and cultured in DMEM/10% FBS..SPL7013 (0, 10 and 30 mg/mL in PBS) was added to Vero E6 cells 1 hour prior to adding SARS-CoV-1, SARS-CoV-2 and MERS-CoV Spike-pseudotyped GFP reporter lentiviral particles (RVP-801, RVP-701 and RVP-901, Integral Molecular) (50 µL). The percent of GFP-positive, or infected, Vero E6 cells was determined by fluorescence-activated cell sorting (FACS) flow cytometry at 48 h post-infection.

Confocal microscopy studies of SARS-CoV-2 spike binding to hACE2+ hTMPRSS2+ 293T cells: hACE2⁺ hTMPRSS2⁺293T cells were cultured onto chamber slides. Cells were treated with SPL7013 (0, 1 mg/mL in PBS) for 1 hour prior to challenge with SARS-CoV-2 spike RBD recombinant protein with an mFc-tag. After 1 hour, cells were washed twice and anti-mFc-PE IgG antibody (1 µg/mL) was added to cells to identify bound spike protein. After 30 min, cells were again washed twice, fixed and analysed by confocal microscopy.

### Results

Assay for SARS-CoV- 1, SARS-CoV-2 and MERS-CoV pseudotyped GFP reporter lentiviral particles: SPL7013 was found to have a broad-spectrum antiviral effect that was specific to the inhibition of the spike protein function at attachment, fusion or both. Pseudotyped lentivirus particles that express antigenically correct spike proteins encoded by SARS-CoV-1, SARS-CoV-2 and MERS-CoV were used to infect Vero E6 cells (FIG. 8B). SARS-CoV-1 and SARS-CoV-2 attach to Vero E6 cells via the ACE2 receptor. MERS-CoV attaches to the dipeptidyl peptidase 4 (DPP4). All three coronaviruses utilize the TMPRSS2 protease to cleave their S1/S2 regions. SPL7013 potently inhibited the binding of pseudotyped lentiviruses expressing the spike protein of SARS-CoV-1, SARS-CoV-2 and MERS-CoV to Vero E6 cells at concentrations of 10 and 30 mg/mL.

Confocal microscopy studies of SARS-CoV-2 spike binding to hACE2 hTMPRSS2⁺ 293T cells: In the confocal microscopy studies, the negative control (no SPL7013 added) showed SARS-CoV-2 spike protein binding to cells expressing the hACE2 receptor on the cell membrane in the absence of SPL7013. This was demonstrated by a strong green immunofluorescence in micrographs showing significant binding of SARS-CoV-2 spike protein to host cells (micrographs not shown). When SARS-CoV-2 was added in the presence of SPL7013, no detectable binding of the SARS-CoV-2 spike protein to the cells expressing the hACE2 receptor was observed. This was indicated by the complete absence of green immunofluorescence in the micrographs (micrographs not shown). These studies confirmed that SPL7013 acts by blocking the SARS-CoV-2 spike protein. The SARS-CoV-2 spike protein is essential in initiating interaction of the virus with the target cell, via the ACE2 receptor, which leads to infection of the cell. In the absence of binding of SARS-CoV-2 spike proteins to cells, infection of cells cannot occur.

The current study with SARS-CoV and MERS-CoV has demonstrated that SPL7013 blocks the binding of the spike proteins at concentrations that demonstrated potency against SARS-CoV-2 spike protein binding and infection. These studies show that SPL7013 acts against all these viruses through a common mechanism of blocking the coronavirus spike protein from interacting with cells, regardless of the cell receptor involved. Collectively, these data support that SPL-7013 has antiviral effect against human pathogenic coronavirus.

### Example 8: Evaluation of Antiviral Properties of SPL7013 Against Respiratory Syncytial Virus (RSV)

The concentrations of the Test Product were prepared using approximately 1:3 dilutions from the starting concentration determined following the cytotoxicity test.

The host cell culture was washed with PBS, and 1.0 mL aliquots of test product dilutions added to the cells. Cells were then incubated for 1 hour ± 15 minutes for equilibration. After the incubation, 1.0 mL aliquots of virus was added to the wells and incubated for 1 hour ± 15 minutes for virus adsorption. Following incubation, the mixture was removed and replaced with TM. The plates were then incubated in a CO₂ incubator. The plates were incubated until viral plaques in the virus control could be microscopically registered (approximately 5-10 days).

For the cytotoxicity control, the host cell culture was washed with PBS. The cells were then overlaid with 1.0 mL of the highest non-toxic test product concentration and incubated for 1 hour ± 15 minutes for equilibration. After the incubation, 1.0 µl aliquots of MM (mock infection) was added to the wells and incubated for 1 hour ± 15 minutes. Following incubation, the mixture was removed and replaced with TM. The plates were then incubated in a CO₂ incubator.

For the virus control, the host cell culture was washed with PBS, and 1.0 mL aliquots of MM (in place of test product) were added to the wells designated for virus control and incubated for 1 hour ± 15 minutes for equilibration. After the incubation was complete, 1.0 mL aliquots of virus were added to the wells and incubated for 1 hour ± 15 minutes for virus adsorption. Following incubation, virus was removed and replaced with TM. The plates were incubated in a CO₂ incubator.

Intact cell culture monolayers were used as the control of cell viability. The GM was replaced by TM in the cell culture control wells.

Following incubation, fixation and staining was performed by removing the TM, washing the plates with PBS and fixed using 4% formaldehyde solution for 4 to 6 hours. Fixed cells were stained using Crystal Violet stain. Unstained zones of cell lysis (viral plaques) were counted.

Antiviral post-treatment test – determination of product cytotoxicity: The highest non-cytotoxic concentration of the test product was determined. The host cell culture was washed with PBS. 1.0 mL aliquots of the test product were added to the cells and incubated in a CO₂ incubator for 24 hours ± 1 hour at 37° C. ± 2° C. Toxicity was evaluated using CCK-8 assay and read with a VERSAmax™ Tunable Microplate Reader at 450 nm. The concentrations used for antiviral test were determined in the cytotoxicity test. Results are presented as percent of cell viability where 100% cell viability is approximately equal to the mean of the cell control. The TC50 concentration of the test product were determined using GraphPad Prism 5.0 statistical software. The antiviral post-treatment test included the procedures outlined in Table 11.

TABLE 11

Antiviral post-treatment test parameters

| Parameter | Summary | Replicates |
|---|---|---|
| Test | Infection of cells with ≤ 100 PFU/mL virus → Inoculation of Product Dilutions → Incubation → Fixation → Staining → Counting of Plaques | 2 |
| Cytotoxicity Control | Inoculation of a Product onto cells → Incubation → Fixation → Staining → Visual assessment | 2 |
| Virus Control | Infection of cells with a virus → Incubation → Fixation → Staining → Counting of Plaques | 2 |
| Cell Culture Control | Maintenance medium → Incubation → Fixation → Staining → Visual assessment | 2 |

The concentrations of the test product were prepared using approximately 1:3 dilutions from the starting concentration determined following the cytotoxicity test.

The host cell culture was washed with PBS, and 1.0 mL aliquots of virus added to the wells and incubated for 1 hour ± 15 minutes for virus adsorption. After incubation, virus inoculum was removed and replaced with aliquots of the test product concentrations in TM. The plates were then incubated in a CO₂ incubator. The plates were then incubated until viral plaques in the virus control can be microscopically registered (approximately 5-10 days).

For the cytotoxicity control, the host cell culture was washed with PBS. The cells were then overlaid with the highest non-toxic test product concentration in TM and incubated in a CO₂ incubator.

For the virus control, the host cell culture was washed with PBS. 1.0 mL aliquots of ≤ 100 PFU/mL virus will be added to the wells and incubated for 1 hour ± 15 minutes for virus adsorption. After incubation, virus inoculum was removed and replaced with aliquots of TM. The plates were incubated in a CO₂ incubator.

For the cell culture control, intact cell culture monolayers were used as the control of cell viability. The GM was replaced by TM in the cell culture control wells. Following incubation, fixing and staining was performed by removing the TM, washing with PBS and fixed using 4% formaldehyde solution for 4 to 6 hours. Fixed cells were stained using Crystal Violet stain. Unstained zones of cell lysis (viral plaques) were counted.

Assessment of antiviral properties: The antiviral properties EC50 and/or EC90 was determined using non-linear regression analysis, GraphPad Prism 5.0 software.

Antiviral test acceptance criteria: A valid test as described in the Example requires that: 1) The plaques in test and control samples are countable; 2) no significant cytotoxic effect is present in cytotoxicity control; 3) cell control wells are viable and attached to the bottom of the well; 4) that the medium is free of contamination in all wells of the plate.

### Results

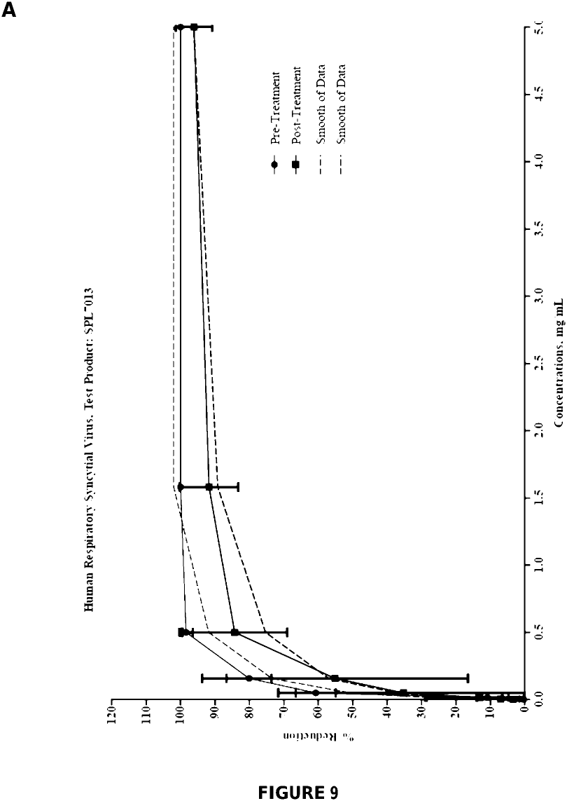

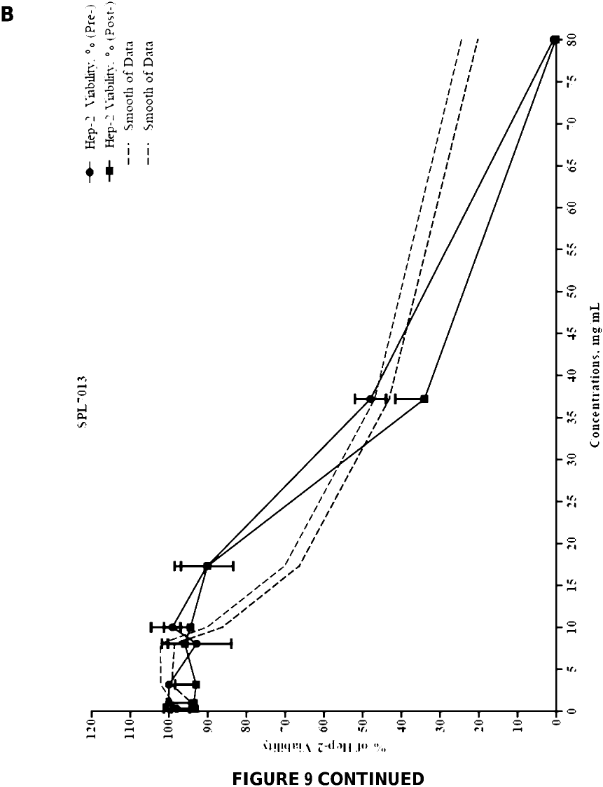

Results are summarized in Table 12 and FIG. 9.

TABLE 12

Summary of results of antiviral screening of SPL7013

| Virus Designation | Product Application | EC50, mg/mL | | EC90, mg/mL | | TC50, mg/mL | | Selectivity Index TC50/EC50 |
|---|---|---|---|---|---|---|---|---|
| | | Best Fit Value | 95% CI | Best Fit Value | 95% CI | Best Fit Value | 95% CI | |
| Human Respiratory Syncytial Virus | PreTreatment | 0.045 | 0.038 to 0.053 | 0.180 | 0.125 to 0.258 | 35.64 | 34.02 to 37.34 | 792 |
| | Post Treatment | 0.107 | 0.064 to 0.181 | 1.159 | 0.368 to 3.649 | 30.90 | 28.69 to 33.28 | 289 |

EC = Effective Concentration; TC = Toxic Concentration; CI = Confidence Interval.

### Example 9: SPL7013 Nasal Spray

An embodiment of the device as described herein is a nasal spray. An embodiment of the nasal spray is provided in this example and referred to as the “SPL7013 Nasal Spray”. The SPL7013 Nasal Spray comprises an aqueous nasal composition containing SPL7013, referred to as the “SPL7013 Nasal Spray Composition”. SPL7013 is intended to inactivate viruses, including SARS-CoV-2 and/or RSV, and to reduce exposure to viral load. Reducing viral load can reduce acquisition or transmission of infection. The SPL7013 Nasal Spray, when comprising the SPL7013 Nasal Spray Composition, produces droplets sizes suitable for administration and delivery to the nasal cavity with less than 5% of droplets being 10 µM or less (particles of 10 µM are more suitable for delivery to the lungs).

Actuations from the SPL7013 Nasal Spray, comprising the SPL7013 Nasal Spray composition, produces a moisturising and protective mucoadhesive barrier for the nasal mucosa that can be used to inactivate and act as a barrier to respiratory viruses.

The SPL7013 Nasal Spray Composition comprises SPL7013 and a viscosity modifying mucoadhesive substance. The SPL7013 Nasal Spray Composition is as described in “Variant 4” listed in Table 14 or “Variant 5” shown below in Table 13.

Variant 5 is the Variant 4 formulation additionally pH adjusted with hydrochloric acid. The formulation comprises a carbomer homopolymer type B to achieve an appropriate viscosity, aid in the ease of administration, and facilitate retention of the product in the nasal cavity.

TABLE 13

SPL7013 Nasal Spray Formulation (variant 5)

| | | |
|---|---|---|
| Component | % w/w | Quantity 1% w/w (kg/ 100 kg batch) |
| Purified water | q.s 100% | 98.51⁺ |
| Astrodrimer sodium (SPL7013) | 1.0 | 1.0+ |
| Propylene glycol | 1.0 | 1.0 |
| Glycerol | 1.16 | 1.0 |
| Methylparaben | 0.18 | 0.18 |
| Carbomer 974P (Carbopol 974P) | 0.05 | 0.05 |
| Propylparaben | 0.02 | 0.02 |
| Edetate disodium dehydrate | 0.005 | 0.005 |
| Sodium hydroxide^ | Adjust pH to 6 | N/A |
| Hyrochloric acid^ | Adjust pH to 6 | N/A |

+ The final amounts of SPL7013 and water are determined by the water content of SPL7013 at the time of manufacture.^ At a concentration of 0.1 N, prepared using purified water (EP).

The SPL7013 Nasal Spray is supplied as a 10 mL multi-dose, metered nasal spray device that delivers ~100 µL of SPL7013 Nasal Spray Composition per actuation. Other embodiments of the SPL7013 Nasal Spray may comprise a slightly smaller or larger volume or smaller or larger metered dose. The SPL7013 Nasal Spray Composition can be self-administered for up to 30 consecutive days by the user as required, and/or up to 4 times daily (in each nostril), as needed, for inactivation of viruses and reduced exposure to viral load.

In reference to the Global Medical Device Nomenclature (GMDN), the term applicable to SPL7013 Nasal Spray is “nasal moisture barrier dressing” with GMDN code 47679. Given the physical nature of the mechanism of viral inactivation by SPL7013, and the physical mode of action of the device, the product is considered a Class 1 medical device under the European Medical Device Directive 93/42/EEC.

SPL7013 Nasal Spray, when comprising the SPL7013 Nasal Spray Composition, upon actuation provides i) a moisturising and protective mucoadhesive formulation which when applied to the nasal mucosa acts as a barrier to respiratory viruses; ii) inactivates viruses and reducing exposure to viral load; iii) as a result of i) and/or ii) reduces viral load. A reduction in viral load may help prevent acquisition or transmission of infection.

The acceptance criteria for the SPL7013 Nasal Spray composition is summarised below. Osmolality and pH of a Nasal Spray Composition are aligned to the physiological situation of <500 mOsmol with a pH of 5.5 - 6.5. The acceptance criteria for SPL7013 Nasal Spray for osmolality and pH are 200-400 mOsmol and 5.5-6.5, respectively. The SPL7013 Nasal Spray typically has a density of ~1 g/mL, suitable for retention in the nasal cavity. The release and expiry acceptance limit for Assay of SPL7013 is 0.80 - 1.20 % w/w. The acceptance criteria for methylparaben and propylparaben are 0.14% - 0.23% and 0.015% - 0.025%, respectively. Any microbial content in SPL7013 Nasal Spray is determined by a Microbial Limits Test, as per Ph. Eur. 2.6.12 Microbiological Examination of Nonsterile Products: Microbial Enumeration Tests and Ph. Eur. 2.6.13 Microbial Examination of Non-Sterile Products: test for Specified Micro-Organisms. Both the total aerobic count and total yeasts and moulds present in the test material are determined using standard pour plate methodology. The specification for the microbial content follows the established limits described in Ph. Eur. 5.1.4 Microbiological Quality of Pharmaceutical Preparations. The specified organisms are based on Ph. Eur 5.1.4, Microbiological Quality of Non-Sterile Pharmaceutical Preparations and Substances for Pharmaceutical Use..

SPL7013 Nasal Spray is packaged as a non-pressurised, compact container-closure system. The container-closure system includes a delivery system (pump with actuator) that administers 100 µL of a spray of droplets of SPL7013 Nasal Spray Composition. The delivery device consists of a pump screwed onto a polyethylene (HDPE) bottle and, the dip tube, housing, gasket and stem of the pump are made from polyethylene polymers. The ball is made from stainless-steel 1.430 and is corrosion resistant. The liner is made of polyoxymethylene.

### Example 10: Biological Evaluation of the SPL7013 Nasal Spray

A comprehensive biological evaluation was conducted for the example SPL2013 Nasal Spray described in Example 9.

The SPL7013 Nasal Spray is a surface device which comes into contact with mucosal membranes (nasal), with prolonged exposure time (>24 hr to 30 days). In accordance with ISO 10993, tests for in vitro cytotoxicity (ISO 10993-5), nasal irritation following repeated administrations in the rat (ISO 10993-10) and skin sensitisation in a guinea pig model (ISO 10993-10) were conducted on SPL7013 Nasal Spray packaged in container-closure system described in Example 9.

In vitro cytotoxicity: The results of the in vitro cytotoxicity study demonstrated that at 5,000 µg/mL, SPL7013 Nasal Spray is not cytotoxic. In a nasal irritation study, rats were administered 100 µL of 1% SPL7013 Nasal Spray in each nostril, four times a day for 14 consecutive days. The results of the study from the in-life phase and the histopathological examinations showed no findings associated with the product and indicate that SPL7013 Nasal Spray is not an irritant.

Skin sensitisation: The skin sensitisation study consisted of a Guinea Pig Maximization Test (GMPT) according to Magnusson and Kligman (1969). The test demonstrated that 1% SPL7013 Nasal Spray is not a sensitiser.

Nasal tolerance and PK: SPL7013 Nasal Spray containing 1% or 3% SPL7013 was also administered nasally 4 times a day (50 µL per nostril) for 7 days to rats to test local toxicity as well as potential for systemic absorption of SPL7013. The study showed that repeated nasal administration of SPL7013 Nasal Spray was well-tolerated and did not cause any clinical signs of local or systemic toxicity. In addition, plasma samples were collected from animals in this study on Study Day 1 before first administration of product and at 15 min, 30 min, 1 hr, 2 hr, 3 hr, 4 hr, 5 hr and 6 hr after first administration, and on Study Day 7 before the last administration of product for the day and at 15 min, 30 min, 1 hr, 2 hr, 3 hr, 4 hr, 5 hr and 6 hr after last administration for the day. Bioanalysis of pooled plasma samples by capillary electrophoresis showed that SPL7013 was not detected at or above the lower limit of quantitation (LLOQ, 0.5 µg/mL) of the assay in any samples from animals administered 1% or 3% SPL7013 Nasal Spray 4 times daily for 7 days, with the exception of one sample from animals in the 1% group, which showed a result just above the LLOQ (0.635 µg/mL) for a 3 hr sample (data not shown). The data indicate that SPL7013 is not absorbed systemically following application to the nasal mucosa in rats following 4 times daily administration for 7 days, and the result for one sample in the lower dose group is an aberration.

### Example 11: SPL7013 Formulation and Testing

SPL7013 (astodrimer sodium) formulations were prepared as described in Table 14.

TABLE 14

SPL7013 example formulations

| Component (%w/w) | Variant 1 | Variant 2 | Variant 3 | Variant 4 | Anticipated activity |
|---|---|---|---|---|---|
| SPL7013 | 1.00 | 1.00 | 1.00 | 1.00 | Active component, physically inactivates the virus |
| Water | 95.50 | 90.00 | 89.00 | 95.52 | Solvent (will vary depending upon pH adjustment) |
| Carbopol 974P | - | - | - | 0.05 | Rheology modifier, viscosity modifier and bio-adhesive |
| Hydroxypropylmethylcellulose | 0.10 | | | | Rheology modifier, viscosity modifier and bio-adhesive |
| Microcrystalline cellulose /carboxy methyl cellulose | | 2.00 | 2.00 | | Rheology modifier, viscosity modifier and bio-adhesive |
| Glycerin/glycerol | 1.00 or 1.16 | 1.00 | 2.30 | 1.00 or 1.16 | Humectant/aids solubility of parabens/tonicity adjustment |
| Propylene glycol | 1.00 | 1.00 | | 1.00 | Humectant/ aids solubility of parabens |
| Methyl paraben | 0.18 | 0.18 | | 0.18 | Preservative |
| Propyl paraben | 0.02 | 0.02 | | 0.02 | Preservative |
| Benzalkonium chloride | | | 0.05 | | Preservative |
| EDTA | 0.005 | 0.005 | 0.005 | 0.005 | Antioxidant/Chelator/ preservative |
| Sodium hydroxide | 1.20 | 1.20 | 1.20 | 1.20 | pH modifier, alkalizing agent |
| Check/adjust pH to | 6.0 | 6.0 | 6.0 | 6.0 | |

Viscosity and Osmolality were measured within a few hours after preparation. Results are provided below in Table 15.

TABLE 15

SPL7013 example formulation characteristics

| | Variant 1 | Variant 2 | Variant 3 | Variant 4 |
|---|---|---|---|---|
| Glycerin (%w/w) | 1.16 | 1.00 | 2.30 | 1.16 |
| Osmolality (mOsmol) | 295 | 283 | 344 | 280 |
| Viscosity (cP) | 2.66 | 107.1 | 110.7 | 1.78 |

To assess the suitability of the formulations for nasal delivery, the formulations were screened using three nasal aerosol pumps and the particle size measured at 30 mm and 60 mm as shown in Table 16.

TABLE 16

SPL7013 example formulations droplet size distribution (DSD) testing

| Formulation | Dispenser 1 (100 ul) | | | | |
|---|---|---|---|---|---|
| | Distance | Dv10 | Dv50 | Dv90 | %vol <10 µm |
| Variant 1 | 30 mm | 20.76 | 66.46 | 155.10 | 2.655 |
| Variant 2 | 30 mm | 18.61 | 59.51 | 126.10 | 3.208 |
| Variant 3 | 30 mm | 18.62 | 58.93 | 130.50 | 3.334 |
| Variant 4 | 30 mm | 14.51 | 42.84 | 97.69 | 5.304 |
| Variant 1 | 60 mm | 23.93 | 57.71 | 136.20 | 2.23 |
| Variant 2 | 60 mm | 21.40 | 51.70 | 112.40 | 2.73 |
| Variant 3 | 60 mm | 20.97 | 47.49 | 100.10 | 3.21 |
| Variant 4 | 60 mm | 16.75 | 42.04 | 92.32 | 4.31 |
| Formulation | Dispenser 2 (100 ul) | | | | |
| | Distance | Dv10 | Dv50 | Dv90 | %vol <10 µm |
| Variant 1 | 30 mm | 11.96 | 30.57 | 76.34 | 7.28 |
| Variant 2 | 30 mm | 10.25 | 27.81 | 69.81 | 9.55 |
| Variant 3 | 30 mm | 11.79 | 31.59 | 81.75 | 7.27 |
| Variant 4 | 30 mm | 10.36 | 28.22 | 72.13 | 9.41 |
| Variant 1 | 60 mm | 14.13 | 33.47 | 66.63 | 6.04 |
| Variant 2 | 60 mm | 13.79 | 31.45 | 62.85 | 5.86 |
| Variant 3 | 60 mm | 14.70 | 33.00 | 69.31 | 5.15 |
| Variant 4 | 60 mm | 13.61 | 30.50 | 59.36 | 6.21 |
| Formulation | Dispenser 3 (100 ul) | | | | |
| | Distance | Dv10 | Dv50 | Dv90 | %vol <10 µm |
| Variant 1 | 30 mm | 9.54 | 25.26 | 59.40 | 10.80 |
| Variant 2 | 30 mm | 9.02 | 22.98 | 48.57 | 11.75 |
| Variant 3 | 30 mm | 9.51 | 24.62 | 55.86 | 10.85 |
| Variant 4 | 30 mm | 9.06 | 21.88 | 45.63 | 11.87 |
| Variant 1 | 60 mm | 12.32 | 30.71 | 62.09 | 7.31 |
| Variant 2 | 60 mm | 15.10 | 30.62 | 53.51 | 5.31 |
| Variant 3 | 60 mm | 12.93 | 29.60 | 54.04 | 6.75 |
| Variant 4 | 60 mm | 12.46 | 28.90 | 52.53 | 7.49 |

These results demonstrate that the formulations provide suitable particle size for intranasal delivery in a variety of nasal pump delivery devices.

Further DSD studies were conducted on the Variant 4 formulation to further investigate particle size at suitable velocities for delivery and are shown below in table 17. Experiments were conducted with a Spraytec Open Spray with a 300 mm lens.

TABLE 17

Variant 4 formulation droplet size distribution (DSD) testing

| | Velocity of actuation: 60 mm/s | | | | Velocity of actuation: 80 mm/s | | | |
|---|---|---|---|---|---|---|---|---|
| | Dv10 | Dv50 | Dv90 | %V<10 µm | Dv10 | Dv50 | Dv90 | %V<10 µm |
| Distance | 30 mm | | | | | | | |
| Mean | 19 | 58.9 | 120.6 | 3.82 | 14.51 | 42.84 | 97.69 | 5.304 |
| %RSD | 10.7 | 11.4 | 9.5 | | - | - | - | |
| Distance | 40 mm | | | | | | | |
| Mean | 30.42 | 89.32 | 180.79 | 1.66 | - | - | - | - |
| %RSD | 15.4 | 13.7 | 10.7 | | - | - | - | |
| Distance | 60mm | | | | | | | |
| Mean | - | - | - | - | 16.75 | 42.04 | 92.32 | 4.31 |
| %RSD | - | - | - | | - | - | - | |
| Distance | 70 mm | | | | | | | |
| Mean | 32.55 | 78.96 | 166.46 | 1.64 | - | - | - | - |
| %RSD | 13.3 | 13.8 | 10.8 | | - | - | - | |

RSD = relative standard deviation.

### Example 12: SPL7013 Hygroscopic Assessment

A hygroscopic assessment was performed as described in European Pharmacopoeia 5.11. Results from the assay are interpreted as follows: deliquescent -sufficient water is absorbed to form a liquid; very hygroscopic - increase in mass is equal to or greater than 15%; hygroscopic - increase in mass is less than 15% and equal to or greater than 2%; slightly hygroscopic - increase in mass is less than 2% and equal to or greater than 0.2% and not hygroscopic - if increase in mass is less than 0.2%, then the compound is not hygroscopic.

Hence, SPL7013 was found to be very hygroscopic in nature with an increase in mass of greater than 15% (21.97%). The hygroscopicity results were confirmed by water content analysis with a Karl Fischer test.

### Example 13: SPL7013 Activity Against SARS-CoV-2 in Primary Human Airway Cells

Primary human bronchial epithelial cells (HBEpC) (Sigma-Aldrich, MO, USA) were grown and maintained in HBEpC/HTEpC growth medium (Cell Applications, CA, USA). These primary cells express the ACE2 receptor and are permissive to SARS-CoV-2 infection. These cells were used to determine the antiviral effect of astodrimer sodium against SARS-CoV-2 in a primary human airway epithelial cell line.

Cells were infected with SARS-CoV-2 2019-nCoV/USA-WAl/2020 at 10³ pfu/mL with 1 mL added to 2.5×10⁴ cells/well. The positive control was addition of 10 µg/mL of SARS-CoV-2 spike protein antibody (pAb, T01KHuRb) (ThermoFisher, MA, USA) at the time of infection. Iota-carrageenan (Sigma-Aldrich, MO, USA) was used in the primary epithelial cell nucleocapsid and plaque assays to compare the antiviral activity of this substance with astodrimer sodium. Concentrations used are those reported to show activity against SARS-CoV-2 (Bansal et al., 2020).

SPL7013 (0, 1.1, 3.3 and 10 mg/mL) or iota-carrageenan (0, 6, 60 and 600 µg/mL) were added to HBEpC cells 1 hour prior to infection with SARS-CoV-2. Cells were cultured for 4 days post-infection and the cell supernatant was analysed for the amount of secreted SARS-CoV-2 nucleocapsid by ELISA, and infectious virus was quantitated by plaque assay, as described in Example 3.

To determine the ability of SPL7013 to prevent SARS-CoV-2 infection of primary human epithelial cells, the compound was evaluated against the 2019-nCoV/USA-WA1/2020 strain in HBEpC cell culture.

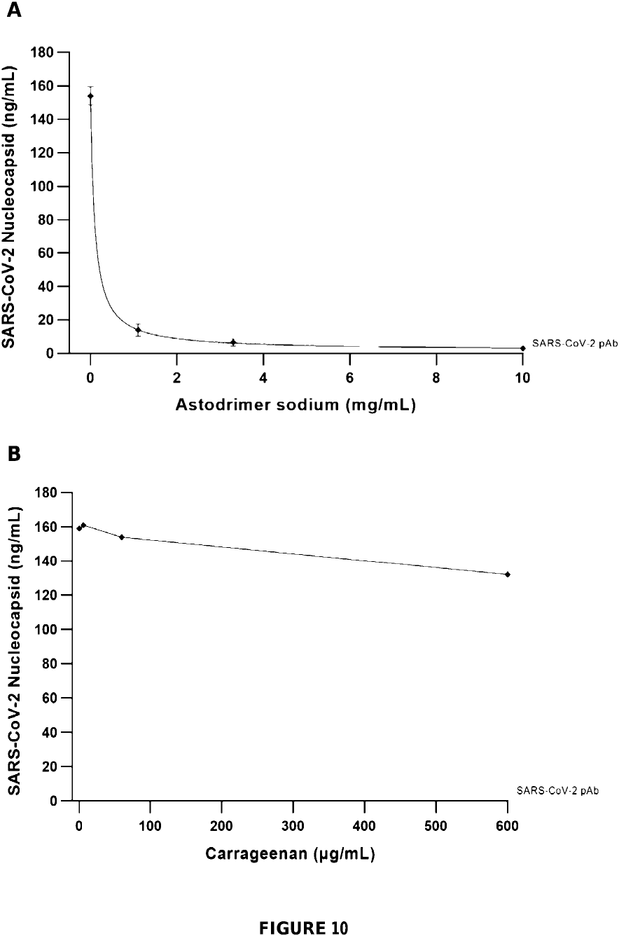

SPL7013 was found to reduce infection of HBEpC primary cells by SARS-CoV-2 by up to 98% vs virus control by nucleocapsid ELISA (FIG. 10A), and by up to 95% in the plaque assay (data not shown). In contrast, treatment with iota-carrageenan had minimal antiviral effect against SARS-CoV-2 in this cell line, with the highest concentration tested reducing infection by just 17% by nucleocapsid ELISA (FIG. 10B), and just 21% in the plaque assay (data not shown). The maximum level of inhibition with astodrimer sodium was comparable to inhibition achieved with the SARS-CoV-2 spike protein antibody (pAb, T01KHuRb) positive control.

Astodrimer sodium inhibited infection of a human airway primary epithelial cell by SARS-CoV-2, whereas iota-carrageenan, which is a polyanionic compound in marketed nasal spray formulations, failed to provide significant inhibition at concentrations that have previously been shown to reduce SARS-CoV-2 infection in Vero E6 cells (Bansal et al., 2020). The unique structure of astodrimer sodium, a sulphonated, roughly spherical molecule with a core and densely packed branches radiating out from the core, appears to provide potential benefits over other polyanionic compounds such as iota-carrageenan and heparin, which are linear sulphated molecules with a distribution of molecular weight. The authors are not aware of data showing that iota-carrageenan is virucidal, while heparin has demonstrated a lack of irreversible, virucidal interaction with HSV virion components (Ghosh et al., 2009).

### Example 14: Rat SPL7013 Biocompatibility Studies

Biocompatibility studies of SPL7013 in formulation variant 4 were conducted in rats (data not shown).

The product was tested to evaluate its cytotoxic effect in Balb/c 3T3 cells. In conclusion, the solution of 5 mg/ml SPL7013 is not cytotoxic.

The product was tested to evaluate its sensitising properties in ten Albino Guinea pigs after intradermal and topical administration followed by challenge after 14 days. In conclusion, no macroscopic cutaneous reactions attributable to allergy were recorded after the challenge phase and the product is not classified as a skin sensitiser in accordance with ISO 10993-10.

The product was administered to 3 female Sprague Dawley rats four times a day for 14 days by the intranasal route at a dose of 0.1 ml in each nostril. No mortality was observed, no clinical signs related to administration of the test product were observed, no erythema or odema was registered on the treatment site and body weight remained normal. There was no evidence of inflammatory change or effect on the epithelia. In conclusion the test product was well tolerated and did not induce any evidence of irritation assessed in accordance with ISO 10993-10.

### Example 15: Clinical Study

A clinical study was conducted in 40 patients receiving 1% SPL7013 in formulation variant 4 or placebo (formulation 4) administered 100µl in each nostril four times a day for 14 days by spray device. No serious adverse events were reported and the formulation was generally well tolerated with minimal irritation.

### Example 16: Summary

The experiments described herein have shown that SPL7013 has demonstrated potent antiviral activity against multiple strains of SARS-CoV-2 and in different cell lines, with very high SI. At the concentration of SPL7013 in the nasal spray (10 mg/mL), the reduction of infectious virus was >5 log₁₀ (>99.999%) in Vero E6 cells and >3 log10 (>99.9%) in Calu-3 cells. Studies examining the kinetics of virucidal activity show that inactivation of SARS-CoV-2 can be observed in a dose-dependent manner with exposure of SPL7013 to virus for as little as 5 seconds.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

This application claims priority from Australian Provisional Application No. 2020901194 entitled “Method of Prophylaxis of Coronavirus infection” filed on 15 Apr. 2020, Australian Provisional Application No. 2020902993 entitled “Method of Prophylaxis of Coronavirus infection” filed on 21 Aug. 2020, and Australian Provisional Application No. 2020904246 entitled “Method of prophylaxis of respiratory syncytial virus infection” filed on 17 Nov. 2020 the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The steps, features, integers, compositions and/or compounds disclosed herein or indicated in the specification of this application individually or collectively, and any and all combinations of two or more of said steps or features.

### REFERENCES

Bansal et al (2020) bioRxiv doi: 10.1101/2020.08.19.225854.

Borchers et al (2013) Clin Rev Allergy Immunol 45(3):331-378 doi: 10.1007/s12016-013-8368-9.

Chandel et al (2019) Biomedicine & Pharmaco doi: 10.1016/j.biopha.2019.108601.

Coronaviridae Study Group of the International Committee on Taxonomy of Viruses (2020) Nature microbiology doi: 10.1038/s41564-020-0695-z.

Foster et al (2020) PNAS doi: 10.1073/pnas.2004999117.

Ghareeb et al (2021) J Pharm Investig doi: 10.1007/s40005-021-00520-4.

Ghosh et al (2009) Glycobiology 19(1), 2-15. doi: 10.1093/glycob/cwn092.

Gordon et al (2020) bioRxiv doi: 10.1101/2020.03.22.002386.

Ibrahim et al (2015) Med Devices (Auckl) doi: 10.2147/MDER.S48888.

Keyaerts et al (2004) Biochem Biophys Res Commun 8;323(1): 264-8.

Kucharski et al (2020) Lancet doi: 10.1016/S1473-3099(20)30144-4.

Magnusson and Kilgman (1969) 52(3):268-76. doi: 10.1038/jid.1969.42.

Pizzorno et al (2020) Antiviral Res doi: 10.1016/j.antiviral.2020.104878.

Melero et al (2013) Curr Top Microbiol Immunol 372: 59-82. doi: 10.1007/978-3-642-38919-1_3.

Ramaekers (2020) Virus Evol 2Jul 24;6(2):veaa052. doi: 10.1093/ve/veaa052.

Reed and Muench (1938) J Hyg 27(3), 493-497.

Shen et al (2020) Clin Infect Dis doi: 10.1093/cid/ciaa203.

Tang et al (2020) Nat Sci Rev doi: 10.1093/nsr/nwaa036.

Umashankar et al (2016) Int Res J of Pharm 7(4):9-16.

van den Worm et al (2012) PLoS ONE 7(3):e32857 doi: 10.1371/journal.pone.0032857.

Verma et al (2014) J der Pharmazie Forschung 2(1): 1-10.

Wang et al (2020) Cell Res 30(3), 269-271 doi: 10.1038/s41422-020-0282-0.

Zarogoulidis et al (2012) Int J Nanomedicine 7:1551-72.

1. A method of preventing or reducing the likelihood of Coronavirus (CoV) infection in a human individual, comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

2. A method of preventing or, reducing the likelihood or severity of a symptom associated with a Coronavirus (CoV) infection in n human individual comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

3. The method of claim 2, wherein the symptom associated with CoV infection is selected from one or more of: fever, cough, sore throat, shortness of breath, viral shedding, respiratory insufficiency, runny nose, nasal congestion, bronchitis, headache, muscle pain, dyspnea, moderate pneumonia, severe pneumonia, and acute respiratory distress syndrome (ARDS).

4. A method of reducing the severity and/or duration of a Coronavirus (CoV) infection in a human individual, comprising,
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

5. A method of treating a Coronavirus (CoV) infection in a human individual comprising:
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

6. A method of preventing or reducing viral shedding in a human individual infected with a Coronavirus (CoV), comprising,
administering to the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

7. A method of reducing transmission of a Coronavirus (CoV) in a human population, comprising:
administering to the respiratory tract of a portion of the population an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 1 to 8 generations with one or more sulfonic acid- or sulfonate-containing moieties attached to one or more surface groups of the dendrimer.

8. A method of preventing or reducing the likelihood of Respiratory syncytial virus (RSV) infection in an individual, comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

9. A method of preventing or, reducing the likelihood or severity of a symptom associated with a Respiratory syncytial virus (RSV) infection in an individual comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

10. The method of claim 7, wherein the symptom associated with RSV infection is selected from one or more of: congested or runny nose, decrease in appetite, coughing, mucus when coughing (yellow, green, or gray mucus), sneezing, sore throat, mild headache, fever, wheezing, rapid breathing or difficulty breathing, bluish colour of the skin (cyanosis), severe asthma symptoms in individuals with asthma, acute bronchitis, severe bronchitis, airway inflammation, airway congestion, chronic obstructive pulmonary disease, heart congestion, bacteraemia, pneumonia, acute otitis media, and recurrent otitis media.

11. A method of reducing the severity and/or duration of a Respiratory syncytial virus (RSV) infection in an individual, comprising,
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

12. A method of treating a Respiratory syncytial virus (RSV) infection in an individual comprising:
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

13. A method of preventing or reducing viral shedding in an individual infected with a Respiratory syncytial virus (RSV), comprising,
administering to the respiratory tract of the individual an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

14. A method of reducing transmission of a Respiratory syncytial virus (RSV) in a population, comprising:
administering to the respiratory tract of a portion of the population an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 1 to 8 generations with one or more sulfonic acid- or sulfonate-containing moieties attached to one or more surface groups of the dendrimer.

15. The method of any one of claims 1 to 7, wherein the CoV is selected from an/a Alphacoronavirus, Betacoronavirus, Gammacoronavirus and Deltacoronavirus.

16. The method of any one of claims 1 to 7 or 15, wherein the CoV is a Betacorinavirus.

17. The method of any one of claims 1 to 7 or 15 or 16, wherein the CoV is selected from: Severe acute respiratory syndrome-related coronavirus-2 (SARS-CoV-2), human coronavirus OC43 (HCoV-OC43), human coronavirus HKU1 (HCoV-HKU1), human coronavirus 229E (HCoV-229E), human coronavirus NL63 (HCoV-NL63), Severe acute respiratory syndrome-related coronavirus (SARS-CoV), and Middle-East respiratory syndrome-related coronavirus (MERS-CoV) or a subtype or a variant thereof.

18. The method of any one of claims 1 to 7 or 15 to 17, wherein the CoV is SARS-CoV-2 or a subtype or variant thereof.

19. The method of any one of claims 8 to 14, wherein the RSV is selected from: RSV subtype A (RSVA) and RSV subtype B (RSVB).

20. The method of any one of claims 1 to 7 or 15 to 18, wherein administering comprises administering topically or administering to the respiratory tract.

21. The method of claim 20, wherein administering topically comprises administering to the hands and/or face.

22. The method of any one of claims 8 to 14 or 20, wherein administering to the respiratory tract comprises administering to the upper respiratory tract and/or lower respiratory tract.

23. The method of claim 22, wherein administering to the upper respiratory tract comprises administering to one or more of the: nasal cavity, oral cavity, sinuses, throat, pharynxlarynx, nasal turbinates, nasopharynx, and oropharynx.

24. The method of claim 22 or claim 23, wherein administering to the upper respiratory tract comprises administering to the nasal mucosa.

25. The method of claim 22, wherein administering to the lower respiratory tract comprises administering to one or more of the: trachea, primary bronchi and lungs.

26. The method of any one of claims 8 to 14, wherein the individual is human.

27. The method of any one of claims 1 to 26, wherein the composition comprises about 0.5% to about 5% by weight of the macromolecule or pharmaceutically acceptable salt thereof.

28. The method of any one of claims 1 to 27, wherein the composition comprises about 1% by weight of the macromolecule or pharmaceutically acceptable salt thereof.

29. The method of any one of claims 1 to 28, wherein the effective amount is about 0.1 to about 5 mg per dose.

30. The method of any one of the claims 1 to 29, wherein the effective amount is about is 1 mg per dose.

31. The method of any one of claims 1 to 30, wherein the macromolecule or a pharmaceutically acceptable salt thereof is administered in a nasal spray or an oral spray.

32. The method of any one of claims 1 to 31, wherein the macromolecule or a pharmaceutically acceptable salt thereof is administered 1 to 8 times daily.

33. The method of any one of claims 1 to 32, wherein the macromolecule or a pharmaceutically acceptable salt thereof is administered for about 1 to about 2 weeks, or about 1 to about 3 weeks, or for less than 30 days.

34. A composition for: preventing or reducing the likelihood of, or treating a Coronavirus (CoV) infection in an individual; reducing the severity and/or duration of CoV infection in an individual; preventing or reducing viral shedding in an individual infected with a CoV; or reducing transmission of a CoV in a population, comprising:
an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

35. The composition of claim 34, wherein the composition is a nasal bioadhesive composition.

36. The composition of claim 34 or claim 35, wherein the composition additionally comprises one or more agents selected from: an antiviral active agent, a vaccine, an immunomodulatory, an antibacterial agent, an anti-inflammatory agent and a nasal bioadhesive agent.

37. The composition of claim 36, wherein the antiviral active agent is selected from one or more of:
i) an antibiotic or a further dendrimer; and
ii) a carrageenan, GM-CSF, IL-6R, CCR5, S protein of MERS, and drugs including, ribavirin, tilorone, favipiravir, Kaletra (lopinavir/ritonavir), Prezcobix (darunavir/cobicistat), nelfinavir, mycophenolic acid, Galidesivir, Actemra, OYA1, BPI-002, Ifenprodil, APN01, EIDD-2801, baricitinib, camostat mesylate, lycorine, Brilacidin, BX-25, an interferon ( e.g. IFNβ), chloroquine and azithromycin.

38. The composition of any one of claims 34 to 37, wherein the composition comprises one or more of: Carbopol 974, hydroxypropylmethyl-cellulose and microcrystalline cellulose /carboxy methyl cellulose.

39. The composition of any one of claims 34 to 38, wherein the composition comprises one or more of: glycerine, propylene glycol, methyl paraben, propyl paraben, benzalkonium chloride, ethylenediamine tetraacetic acid and sodium hydroxide.

40. The composition of any one of claims 34 to 39, wherein the composition comprises:
(a) a rheology modifier selected from one or more of: Carbopol 974, hydroxypropylmethyl-cellulose or microcrystalline cellulose /carboxy methyl cellulose;
(b) a preservative selected from one or more of: methyl paraben, propyl paraben, and benzalkonium chloride
(c) an excipient selected from one or more of: glycerine, propylene glycol, ethylenediamine tetraacetic acid; and
(d) a pH modifier.

41. The composition of anyone of claims 38 to 40, wherein the composition comprises a w/w ratio of about 1:20 to 1:10 of Carbopol 974 or Carbopol 971 to the macromolecule.

42. The composition of anyone of claims 38 to 41, wherein the composition comprises about 0.05% w/w Carbopol 974 or Carbopol 971 and about 1% w/w of macromolecule.

43. The composition of any one of claims 34 to 42, wherein the composition is in a form selected from: a liquid, semi-solid, solid, and powder composition.

44. The composition of any one of claims 34 to 43, wherein the composition has a pH of about 3.5 to about 7.5, or a pH of about 5.5. to about 6.5.

45. The composition of any one of claims 34 to 44, wherein the composition has a viscosity of about 1 to about 10 cP.

46. The composition of any one of claims 34 to 45, wherein the composition is suitable for administration in a device selected from the group consisting of: a nasal spray, an oral spray, an inhaler, a nebuliser, nasal wash or oral wash.

47. The composition of claim 46, wherein the spray, inhaler or nebuliser comprises a means for generating particles of a size of about 0.1 µm to about 100 µm.

48. The composition of claim 47, wherein less than 6% of the particles are of a size of about 10 µm or less.

49. The composition of claim 47 or claim 48, wherein particle size is measured using an actuation of 60 mm/s and a distance of 40 to 70 mm from the means for generating particles.

50. The composition of any one of claims 34 to 49, wherein the composition comprises: SPL7013, water, Carbopol 974 or Carbopol 971, hydroxypropylmethyl-cellulose, microcrystalline cellulose, glycerin, propylene glycol, methyl paraben, propyl paraben, benzalkonium chloride, and EDTA.

51. The composition of any one of claims 34 to 50, wherein the composition is present in or applied to protective wear or cleaning products.

52. The composition of claim 51, wherein the protective wear is selected from a face mask, gloves and a gown.

53. The composition of claim 51, wherein the cleaning product is selected from a wipe, a surgical field preparation spray or a cleaning solution.

54. The composition of any one of claims 34 to 53, wherein the composition inactivates more than 90%, or more than 92%, or more than 95%, or more than 99%, or more than 99.9% of SARS-CoV2 or a subtype or variant thereof.

55. The composition of claim 54, wherein the composition inactivates more than 90%, or more than 92%, or more than 95%, or more than 99% or more than 99.9% of SARS-CoV2 or a subtype or variant thereof after at least 1 minute of exposure to the virus.

56. The method of any one of claims 1 to 33, or the composition of any one of claims 34 to 55, wherein the macromolecule or pharmaceutically acceptable salt thereof is a dendrimer comprising lysine building units of from 3 to 5 generations, and the sulfonic acid- or sulfonate-containing moieties are napthyldisulfonate moieties.

57. The method of any one of claims 1 to 33 or 54, or the composition of any one of claims 34 to 56, wherein the sulfonic acid- or sulfonate-containing moiety is selected from the group consisting of: 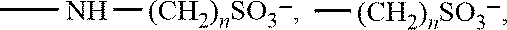 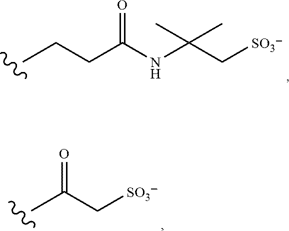 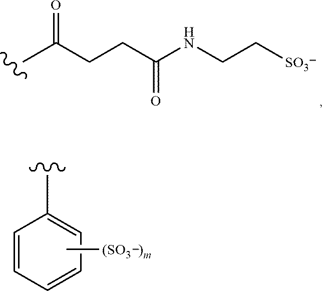
and 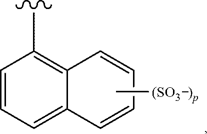
wherein n is zero or is an integer from 1-20, m is an integer 1 or 2 and p is an integer 1 to 3.

58. The method or composition of claim 57, wherein the sulfonic acid or sulfonate-containing moiety is selected from the group consisting of.

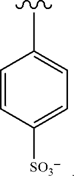

59. The method or composition of claim 57 or claim 58, wherein the sulfonic acid-containing moiety is.

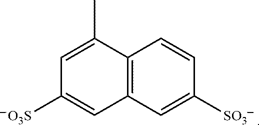

60. The method of any one of claims 1 to 33 or claims 56 to 59, or the composition of any one of claims 34 to 59, wherein the sulfonic acid- or sulfonate- containing moiety is attached to the dendrimer terminal amino group by a linker.

61. The method or composition of claim 60, wherein the linker is an alkylene or alkenylene group in which one or more non-adjacent carbon atoms is optionally replaced with an oxygen or sulfur atom, or a group —X₁—(CH₂)_{q}—X₂— wherein X₁ and X₂ are independently selected from —NH—, —C(O)—, —O—, —S—, and —C(S) and q is 0 or an integer from 1 to 10, and in which one or more non-adjacent (CH₂) groups may be replaced with —O— or —S—.

62. The method or composition of claim 61, wherein the linker is #—O—CH₂—C(O)—* in which # designates attachment to the sulfonic acid-containing moiety and * designates attachment to the terminal amino group of the dendrimer.

63. The method of any one of claims 1 to 33 or claims 56 to 62, or the composition of any one of claims 34 to 62, wherein the dendrimer has 3-4 generations.

64. The method of any one of claims 1 to 33 or claims 56 to 63, or the composition of any one of claims 34 to 63, wherein the dendrimer is a polylysine dendrimer.

65. The method or composition of claim 64, wherein the dendrimer is 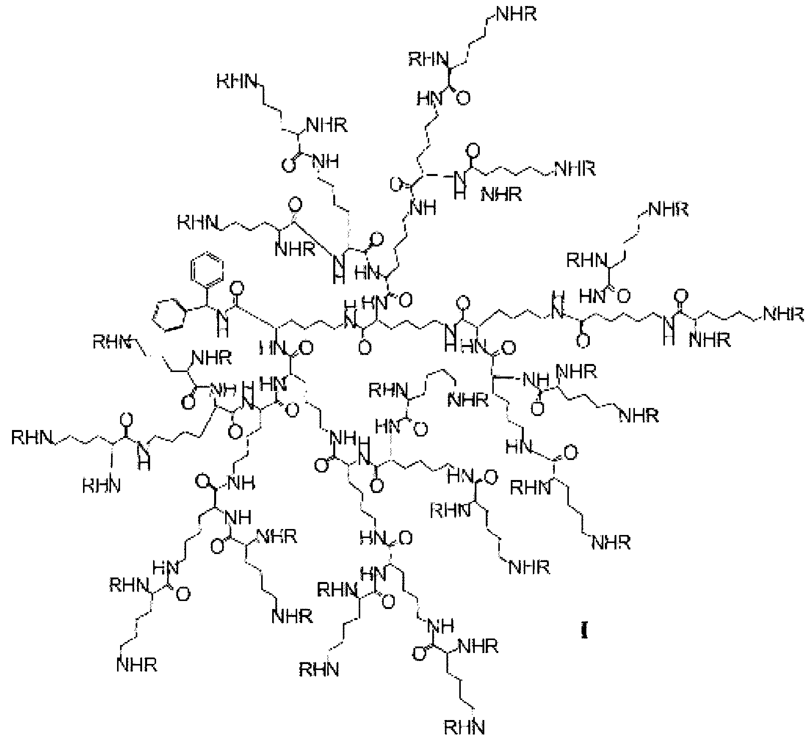
and wherein at least 50% of R is 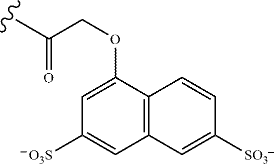
, and wherein the pharmaceutically acceptable salt is a sodium salt.

66. A device for delivering a nasal, oral or pulmonary composition comprising a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

67. The device of claim 66, wherein the device is a nasal delivery device or an oral delivery device for delivering a spray.

68. The device of claim 66, wherein the device is an inhaler or a nebuliser.

69. A nasal moisture barrier dressing comprising a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer.

70. A composition comprising:
an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 974 or Carbopol 971,
wherein the composition comprises a w/w ratio of about 1:20 to about 1:10 of Carpobol 974 or Carbopol 971 to the macromolecule.

71. A composition comprising:
an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 974,
wherein the composition comprises about 0.05% w/w to about 5% w/w, or about 0.05% w/w to about 3% w/w, or about 0.05% w/w to about 2% w/w, or about 0.05% w/w to about 1% w/w, or about 0.05% w/w Carbopol 974.

72. A composition comprising:
an effective amount of a macromolecule or a pharmaceutically acceptable salt thereof, or a composition comprising the macromolecule or pharmaceutically acceptable salt thereof and a pharmaceutically acceptable carrier,
wherein the macromolecule comprises a dendrimer of 3 to 5 generations with one or more sulfonic acid- or sulfonate-containing moieties attached to one or more surface groups of the dendrimer and Carbopol 971,
wherein the composition comprises about 0.05% w/w to about 1% w/w, or about 0.05% w/w to about 1.5% w/w, or about 0.05% w/w to about 1.8% w/w Carbopol 971.
